(19) United States
(12) Patent Application Publication
(10) Pub. No.: US 2025/0115926 A1
(43) Pub. Date: Apr. 10, 2025

(54) DEREGULATED DHS AND TYRA ENZYMES OF GRASSES ENABLE EFFICIENT PRODUCTION OF BOTH TYROSINE AND PHENYLALANINE

(71) Applicant: Wisconsin Alumni Research Foundation, Madison, WI (US)

(72) Inventors: Hiroshi Maeda, Madison, WI (US); Jorge El Azaz, Madison, WI (US)

(21) Appl. No.: 18/906,694

(22) Filed: Oct. 4, 2024

Related U.S. Application Data
(60) Provisional application No. 63/588,272, filed on Oct. 5, 2023.

(51) Int. Cl. C12N 15/82

(52) U.S. Cl. CPC C12N 15/8251

(57) ABSTRACT

The present invention provides engineered cells and plants that express deregulated aromatic amino acid synthesis pathway enzymes from grasses. Methods for increasing the production of aromatic amino acids and their derivatives in cells and plants by engineering them to express these enzymes and methods for producing aromatic amino acids or derivatives thereof and/or sequestering carbon dioxide by growing the plants are also provided.

### CROSS-REFERENCE TO RELATED APPLICATIONS

This application claims priority to U.S. Provisional Application No. 63/588,272 filed on Oct. 5, 2023, the contents of which are incorporated by reference in their entireties.

### STATEMENT REGARDING FEDERALLY SPONSORED RESEARCH

This invention was made with government support under 1836824 awarded by the National Science Foundation. The government has certain rights in the invention.

### SEQUENCE LISTING

This application includes a sequence listing in XML format titled “960296_04546_ST26.xml”, which is 129,604 bytes in size and was created on Sep. 16, 2024. The sequence listing is electronically submitted with this application via Patent Center and is incorporated herein by reference in its entirety.

### BACKGROUND

Plants can directly convert atmospheric carbon dioxide (CO₂) into diverse aromatic natural products, which are primarily derived from the aromatic amino acids tyrosine, phenylalanine, and tryptophan. Aromatic compounds have unusual stability due to their aromaticity (i.e., electron delocalization). As a result, aromatic compounds have potential to be used as a carbon sink for reducing atmospheric CO₂. Aromatic compounds are also key precursors for pharmaceuticals, commodity chemicals, and industrial materials, for which there is rapidly growing global demand. However, the chemical conversion of CO₂ into aromatic compounds remains challenging, and fossil fuels remain the primary source of aromatic compounds. Thus, there remains a need in the art for improved methods for harvesting aromatic compounds from renewable sources, such as plants.

### SUMMARY

In a first aspect, the present invention provides cells engineered to express or overexpress a deregulated enzyme selected from: (a) a DHS1b enzyme comprising SEQ ID NO: 3 (BdDHS1b), SEQ ID NO: 19 (OsDHS1b), SEQ ID NO: 27 (SvDHS1b) or a DHS1b enzyme having at least 95% identity to one of SEQ ID NOs: 3, 19 or 27; or (b) a noncanonical TyrA enzyme comprising SEQ ID NO: 37 (BdTyrAnc), SEQ ID NO: 43 (SbTyrAnc) or a TyrA enzyme having at least 95% identity to SEQ ID NO: 37 or 43. In some embodiments, the cells are plant cells.

In a second aspect, the present invention provides plants comprising the engineered cells described herein.

In a third aspect, the present invention provides methods for increasing production of one or more aromatic amino acids in a cell. The methods comprise engineering the cell to express or overexpress an enzyme selected from: (a) a DHS1b enzyme comprising SEQ ID NO: 3 (BdDHS1b), SEQ ID NO: 19 (OsDHS1b), SEQ ID NO: 27 (SvDHS1b) or a DHS1b enzyme having at least 95% identity to one of SEQ ID NOs: 3, 19 or 27; or (b) a noncanonical TyrA enzyme comprising SEQ ID NO: 37 (BdTyrAnc), SEQ ID NO: 43 (SbTyrAnc) or a TyrA enzyme having at least 95% identity to SEQ ID NO: 37 or 43. In some embodiments, the cell is engineered to express an enzyme that is not native to the cell. In other embodiments, the cell is engineered to overexpress an enzyme that is native to the cell as compared to a control cell. In some embodiments, the cell is a plant cell.

In a fourth aspect, the present invention provides methods for using the plants described herein to (1) produce aromatic amino acids or derivatives thereof, or (2) sequester CO₂. Both sets of methods comprise growing the plants described herein. The methods for producing aromatic amino acids or derivatives thereof further comprise purifying the aromatic amino acids or derivatives thereof produced by the plant.

### BRIEF DESCRIPTION OF THE DRAWINGS

FIGS. 1A-1E show results of a ¹³CO₂ feeding experiment that demonstrate that tyrosine is synthesized at high levels in Brachypodium distachyon. FIG. 1A: Schematic representation of plant aromatic amino acid biosynthesis pathways and feedback regulation (red lines). The grass-specific, bifunctional enzyme phenylalanine tyrosine ammonia-lyase (PTAL, blue) constitutes a shortcut in the phenylpropanoid pathway, transforming tyrosine into p-coumaric acid in a single step. Dashed arrows indicate multiple enzymatic steps. Enzyme abbreviations: TyrA, arogenate dehydrogenase; ADT, arogenate dehydratase; PAL, phenylalanine ammonia lyase; C4H, cinnamate 4-hydroxylase; 1, prephenate dehydrogenase TyrAₚ (EC 4.2.1.91), only found in legumes; 2, prephenate dehydratase, a side activity of plant ADT enzymes; 3, aromatic amino acid aminotransferase. FIG. 1B: Schematic representation of the ¹³CO₂ feeding circuit. FIGS. 1C-1E: Graphs showing the total content per gram of fresh weight (left panel), content of ¹³C-labeled metabolite (central panel), and relative ¹³C-labeled metabolite over the total content (right panel) for tyrosine (FIG. 1C), phenylalanine (FIG. 1D), and shikimate (FIG. 1E). The levels in 4-week-old Brachypodium distachyon (turquoise, triangles) and Arabidopsis thaliana (magenta, circles) plants are compared. All data points are means±SD of n=2. *P<0.05, according to Student's t-test (two-sided test for two samples with equal variance).

FIGS. 2A-2C show graphs comparing tyrosine (FIG. 2A), phenylalanine (FIG. 2B), and shikimate (FIG. 2C) production in ¹³C-labeled leaves and stems, and reveals that grass stems, unlike Arabidopsis stems, maintain a high rate of both tyrosine and phenylalanine production. Total and ¹³C-labeled phenylalanine, tyrosine, and shikimate detected in leaves (pale green circles) and developing stems (dark blue squares) of 6-week-old Brachypodium distachyon, Arabidopsis thaliana, and Setaria viridis plants. All data points are means±SD of n=2. *P<0.05, according to Student's t-test (two-sided test for two samples with equal variance).

FIGS. 3A-3F demonstrate that grass TyrA enzymes differ in their transcriptional and biochemical regulation. FIG. 3A: Phylogenetic tree of plant TyrA enzymes (outgroup: Chlamydomonas reinhardtii) highlighting three clades of grass TyrA enzymes: TyrA1, TyrA2, and non-canonical TyrA (TyrAnc). The enzymes highlighted in blue text were characterized in this study. Bootstrap test values (based on 1000 replications) below 50 have been omitted. The scale bar indicates the number of amino acid substitutions per site. Species abbreviations: AH, Amaranthus hypochondriacus; Aco, Ananas comosus v3; At, Arabidopsis thaliana; EL, Beta vulgaris EL10_1.0; Cre, Chlamydomonas reinhardtii; Bd, Brachypodium distachyon; HORVU, Hordeum vulgare; Mapoly, Marchantia polymorpha; Medtr, Medicago truncatula; Oropetium, Oropetium thomaeum, LOC_Os, Oryza sativa; Pavir, Panicum virgatum; Phvul, Phaseolus vulgaris; Potri, Populus trichocarpa; selmo, Selaginella moellendorffii; Sevir, Setaria viridis; Solyc, Solanum lycopersicum; Spov, Spinacia oleracea; Sb, Sorghum bicolor; Zm, Zea mays; Zosma, Zostera marina. FIG. 3B: Heatmaps showing expression patterns of TyrA and PTAL genes in Brachypodium distachyon (top) and Sorghum bicolor (bottom) across different organs and developmental stages. The levels of expression are relative to the average abundance for each individual gene. Data were obtained from the databases PlaNet⁴¹ and MOROKOSHI⁴² for Brachypodium and Sorghum, respectively. FIG. 3C: RT-qPCR results from an analysis of TyrA transcript abundance in young leaves and internodes from Brachypodium distachyon (left panel) and Setaria viridis (right panel). Error bars=SD; n=3; *P<0.05, **P<0.01, ***P<0.001, ns=not significant (P>0.05), according to Student's t-test (two-sided test for two samples with equal variance). FIGS. 3D-3E: Graphs showing the in vitro sensitivity to feedback-inhibition by tyrosine of the recombinant TyrA enzymes of Brachypodium (FIG. 3D) and Sorghum (FIG. 3E) (error bars=SD; n=4 from two independent experiments). FIG. 3F: Graph showing tyrosine content per gram of fresh weight at 72 hours following Agrobacterium-mediated transient expression of different TyrA genes in the leaves of Nicotiana benthamiana. Arabidopsis AtTyrA2, beet BvTyrAαi, and GUS were expressed as side-by-side controls. Letters indicate significant differences between treatments according to Student's t-test (P<0.05; two-sided test for two samples with equal variance). Error bars=SD; n=6.

FIGS. 4A-4F demonstrate that grasses have a feedback-insensitive DHS1b enzyme that is expressed in internodes. FIG. 4A: Phylogenetic tree of plant DHS enzymes (outgroup: Marchantia polymorpha). Enzymes highlighted in blue text were characterized in this study. Bootstrap test values (based on 1000 replications) below 50 have been omitted. Scale bar indicates number of amino acid substitutions per site. Additional species abbreviations that were not included in FIG. 3A are: Cucsa, Cucumis sativus; Gorai, Gossypium raimondii, and GSMUA, Musa acuminata. FIG. 4B: Heatmap showing expression patterns of Brachypodium DHS genes in different organs and developmental stages compared to BdTyrA1, BdPTAL, and other genes of the lignin pathway. Enzyme abbreviations: 4CL, 4-coumarate: CoA ligase; C3′H, 4-Coumarate 3-hydroxylase; and COMT, caffeic acid/5-hydroxyferulic acid O-methyltransferase. FIG. 4C: Graph showing the inhibition of recombinant Brachypodium BdDHS1a, BdDHS1b, and BdDHS2 by 0.5 mM of aromatic amino acids (error bars=SD; n=3). FIG. 4D: Graph showing the IC₅₀ determination curve of tryptophan inhibition on the Brachypodium DHS enzymes. FIG. 4E: Graph showing the effect of 0.5 mM of AAA pathway intermediates on Brachypodium DHS enzymes (error bars=SD; n=3). FIG. 4F: Graph showing the IC₅₀ determination curve of arogenate inhibition on the Brachypodium DHS enzymes. Asterisks indicate significant differences compared to the water control according to Student's t-test (P<0.01; two-sided test for two samples with equal variance).

FIGS. 5A-5B show that co-expression of DHS1b and TyrA1 in Nicotiana benthamiana has a synergistic impact on tyrosine production while maintaining high phenylalanine accumulation. FIG. 5A: Graphs showing the levels of phenylalanine, tyrosine, tryptophan, and their common intermediate shikimate, three days after the transient expression of Brachypodium DHS1a, DHS1b, and DHS2 in the leaves of Nicotiana benthamiana under control of a RuBisCO promoter. Asterisks indicate significant differences compared to the tdTomato (tdTom) negative control according to Student's t-test (P<0.05; two-sided test for two samples with equal variance). Error bars=SD; n=5. FIG. 5B: Impact of the expression of BdDHS1b or BdDHS2 together with BdTyrA1, BdTyrA2, or BdTyrAnc on tyrosine (top) and phenylalanine (bottom) accumulation in Nicotiana benthamiana leaves. Level 2 Golden Gate vectors were assembled to ensure the simultaneous expression of the different DHS and TyrA genes. Letters indicate significant differences between treatments according to Student's t-test (P<0.05; two-sided test for two samples with equal variance). Error bars=SD; n=6.

FIGS. 6A-6B show the ¹³C-isotopologue abundance of phenylalanine (left), tyrosine (center) and shikimate (right) in Arabidopsis thaliana (FIG. 6A) and Brachypodium distachyon (FIG. 6B) as detected in the ¹³CO₂ feeding experiment described in FIG. 1. Solid blue lines represent the concentration of unlabeled metabolite, dashed red-pink lines correspond to different ¹³C isotopologues. The other possible isotopologues were undetectable or only detectable as a trace, so were not represented in these graphs. 1×¹³C isotopologues, which are naturally abundant, were not quantified for the study. Error bars=SD; n=2 for each individual time point.

FIGS. 7A-7C show the ¹³C-isotopologue abundance of phenylalanine (left), tyrosine (center) and shikimate (right) in leaves and stems of Arabidopsis thaliana (FIG. 7A), Brachypodium distachyon (FIG. 7B), and Setaria viridis (FIG. 7C) as detected in the ¹³CO₂ feeding experiment described in FIG. 2. Isotopologues with only one ¹³C atom, which are naturally present at high abundance, were not represented. Error bars=SD; n=2 for each individual time point.

FIG. 8 shows transient expression of TyrA-EGFP fusion proteins in Arabidopsis protoplasts. Laser scan confocal microscopy of Arabidopsis protoplasts expressing the full-length CDS of Brachypodium distachyon BdTyrA enzymes fused to EGFP. AtTyrA2-EGFP was used as positive control for plastidial localization. Free EGFP was used as positive control for cytosolic localization.

FIGS. 9A-9C are graphs showing the original Ct values and calibration curves for the measurement of TyrA expression in the leaves and developing internodes of the grasses Brachypodium distachyon and Setaria viridis by RT-qPCR. Ct values in Brachypodium distachyon (FIG. 9A) and Setaria viridis (FIG. 9B) (Error bars=SD; n=3). BdUBI10 (UBIQUITIN LIGASE 10) and SvUBI4 (UBIQUITIN LIGASE 4) were used as reference genes. Standard curves were generated for each individual TyrA amplicon (FIG. 9C) (n=2).

FIGS. 10A-10B are graphs showing the activity of pure recombinant TyrA enzymes from Brachypodium (FIG. 10A) and Sorghum (FIG. 10B) in the presence of different substrates (arogenate or prephenate) and electron acceptors (NAD⁺ or NADP⁺). Substrates and cofactors were tested at a concentration of 1 mM. Enzyme concentration was increased up to 10-times when using prephenate as a substrate or NAD⁺ as an acceptor to increase the assay's sensitivity. Error bars=SD; n=3; n.d.=not detected.

FIGS. 11A-11B show Michaelis-Menten and tyrosine inhibition plots for grass TyrA enzymes. FIG. 11A: Michaelis-Menten plots corresponding to the kinetic parameters (Kₘ and Vₘₐₓ) shown in Table 1. FIG. 11B: Tyrosine-inhibition plots used to calculate the IC₅₀ values shown in Table 1. Individual points represent the average of 4 to 6 datapoints coming from at least two independent experiments conducted on different days using different batches of purified recombinant enzyme. Error bars=SD.

FIGS. 12A-12C show inhibition kinetics for BdTyrA1 and BdTyrAnc. Michaelis-Menten (left panel) and Lineweaver-Burk (right panel) plots for BdTyrA1 (FIG. 12A) and BdTyrAnc (FIG. 12B), assayed at two alternative concentrations of tyrosine (legends are shown in the right panels). FIG. 12C: Apparent Kₘ and Vₘₐₓ values, calculated from the data shown in FIG. 12A and FIG. 12B. Individual points represent the average of two technical replicates from the same experiment. Error bars=SD.

FIGS. 13A-13C show schematic representations of the Golden-Gate plant expression constructs used in this study for transient expression of TyrA and DHS enzymes in Nicotiana benthamiana. FIG. 13A: Level 2 vector for the expression of grass TyrA enzymes under the control of the CaMV 35S promoter. AtTyrA2, GUS, and BvTyrAα were used as controls. FIG. 13B: Level 1 vector for the expression of Brachypodium DHS enzymes as HA-tagged proteins under the control of the Arabidopsis AtRbcS3B promoter. The fluorescent protein tdTomato was used as negative control. FIG. 13C: Level 2 assemblies of TyrA-YPet and DHS-HA level 1 vectors, with their corresponding controls. The P19 repressor of the RNA silencing machinery was cloned in position 1 under control of the Arabidopsis ubiquitin ligase promoter, as it was found to be important for enhancing the expression level of BdDHS1b and BdDHS2. Yellow boxes represent Golden Gate overhangs used to assemble the constructs. Abbreviations: PhcTP, plastid transit peptide of Petunia×hybrida 5-enol-pyruvyl-shikimate-3-phosphate synthase (Della-Cioppa et al., 1986); RSSUcTP, plastid transit peptide of Arabidopsis RuBisCO Small Subunit; EU+NbHSP terminator was based on Diamos A G, and Mason H S (2018); NOS, Agrobacterium tumefaciens nopaline synthase; and OCS, Agrobacterium tumefaciens octopine synthase.

FIGS. 14A-14C show Michaelis-Menten plots for Brachypodium DHS enzymes. Plots are shown for the substrates phosphoenolpyruvate (PEP, left) and erythrose 4-phosphate (E4P, right) for the enzymes BdDHS1a (FIG. 14A), BdDHS1b (FIG. 14B), and BdDHS2 (FIG. 14C). Data points represent the average of at least two replicates from independent experiments using different preparations of purified recombinant enzyme. Error bars=SD.

FIGS. 15A-15C are graphs showing the effect of AAAs and related compounds on the activity of recombinant Brachypodium DHS enzymes (validation of the results shown in FIGS. 4C and 4E). FIG. 15A: Modulation of Brachypodium DHS enzyme activity by products of AAA catabolism and lignin biosynthesis intermediates, determined by measuring phosphoenolpyruvate consumption at 232 nm. FIGS. 15B-15C: Effect of AAAs (FIG. 15B) and AAA biosynthesis intermediates (FIG. 15C) on Brachypodium DHS enzyme DAHP production, determined by UHPLC-MS. All compounds were tested at a fixed concentration of 0.5 mM. Error bars=SD; n=3.

FIGS. 16A-16B show the results of DHS inhibition assays using hydrolyzed arogenate as control and the effect of NaCl concentration on DHS activity. FIG. 16A: Arogenate that is hydrolyzed to phenylalanine via treatment with 1N HCl for 30 minutes (Zamir et al., 1980) does not inhibit Brachypodium DHS enzymes or Arabidopsis thaliana AtDHS2, which was also previously reported to be inhibited by arogenate (Yokoyama et al., 2021). FIG. 16B: Increasing concentrations of NaCl result in increased activity of recombinant Brachypodium DHS enzymes. This is likely the cause of the increase in BdDHS1b activity observed when using high concentrations of arogenate, such as in FIG. 4F. Error bars=SD; n=3.

FIGS. 17A-17B show the effects of AAAs and related compounds on the activity of recombinant Sorghum DHS enzymes. FIG. 17A: Effect of AAAs determined based on phosphoenolpyruvate consumption. FIG. 17B: Effect of AAA intermediates determined based on phosphoenolpyruvate consumption. All compounds were tested at a fixed concentration of 0.5 mM. Error bars=SD; n=3.

FIGS. 18A-18E show inhibition kinetics of BdDHS2 in the presence of tryptophan and arogenate. FIGS. 18A-18D: Michaelis-Menten plots showing the activity of BdDHS2 in the presence of various concentrations of phosphoenolpyruvate (PEP) and tryptophan (FIG. 18A), PEP and arogenate (FIG. 18B), erythrose 4-phosphate (E4P) and tryptophan (FIG. 18C), and E4P and arogenate (FIG. 18D). FIG. 18E: Apparent kinetic parameters calculated from the data shown in FIGS. 18A-18D. Hill equation was used to fit E4P kinetic data. Individual points represent the average of at least two to three technical replicates from the same experiment. Error bars=SD.

FIG. 19 shows the effect of various combinations of the effectors tryptophan (Trp), tyrosine (Tyr), chorismate (Cha), and arogenate (Agn) on the activity of recombinant Brachypodium DHS enzymes. All effectors were tested at a concentration of 0.15 mM each. Error bars=SD; n=3; n.d.=not detected.

FIGS. 20A-20B show an immunoblot quantification of the abundance of recombinant Brachypodium DHS enzymes (which comprised a C-terminal HA tag) in Nicotiana benthamiana. FIG. 20A: Anti-HA immunoblot (top) and Coomassie blue staining of total protein as loading control (bottom) in samples from five independent plants. The expected sizes of the mature full-length proteins were: BdDHS1a, 62.4 kDa; BdDHS1b, 63.4 kDa; and BdDHS2, 60.7 kDa. A calibration curve for the HA tag quantification was generated using pure recombinant BdDHS1b-HA protein mixed with total protein extract from a non-infiltrated leaf of Nicotiana benthamiana. The arrow on the right indicates the expected size of the full-length BdDHS1b-HA protein. Bands of lower molecular weight in the BdDHS1b-HA standard were presumed to be degradation products and were not considered for quantitative purposes. All immunoblot images were non-saturated, and all shown membranes were exposed simultaneously. Vertical lines separate independent membranes/gels. FIG. 20B: Graph showing DHS protein abundance by gram of fresh weight of plant sample, based on anti-HA immunoblot signal. P values according to Student's t-test for two samples with equal variance. Error bars=SD; n=5.

FIG. 21 shows graphs that demonstrate that transient expression of BdDHS1b and OsDHS1b in planta results in increased accumulation of aromatic amino acids and their precursors. The levels of phenylalanine, tyrosine, tryptophan, their common intermediate shikimate, and the phenylalanine and tyrosine precursors prephenate and arogenate, were determined by LCMS in the leaves of Nicotiana benthamiana at three days post-infiltration with constructs encoding the Brachypodium distachyon enzymes BdDHS1a and BdDHS1b, the Oryza sativa enzyme OsDHS1b, and the Sorghum bicolor enzyme SbDHS1b under control of the RuBisCO S3B promoter from Arabidopsis thaliana. Note that different scales have been used for each individual graph. Data are presented as the average of n=4 individual plants; error bars=SE. Significant P values (<0.05) with respect to the YPet negative control, calculated using the Mann-Whitney U test, are shown next to the corresponding bar. n.d.=not detected.

FIG. 22 shows graphs that demonstrate that grass DHS enzymes other than BdDHS1b and OsDHS1b do not trigger in planta accumulation of aromatic amino acids. The levels of phenylalanine, tyrosine, tryptophan, shikimate, prephenate, and arogenate, were determined by LCMS in the leaves of Nicotiana benthamiana at three days post-infiltration with constructs encoding the four DHS isoforms (i.e., DHS1a, DHS1b, DHS2, and DHSnc) of Brachypodium dystachion (Bd), Oryza sativa (Os), Sorghum bicolor (Sb), and Setaria viridis (Sv) under control of the RuBisCO S3B promoter from Arabidopsis thaliana.⁵² Note that different scales have been used for each individual graph. Data are presented as the average of n=5 individual plants; error bars=SE. Significant P values (<0.05) with respect to the YPet negative control, calculated using the Mann-Whitney U test, are shown above the corresponding bar. n.d.=not detected.

### DETAILED DESCRIPTION

The present invention provides engineered cells and plants that express deregulated aromatic amino acid synthesis pathway enzymes from grasses. Methods for increasing the production of aromatic amino acids and their derivatives in cells and plants by engineering them to express these enzymes and methods for producing aromatic amino acids or derivatives thereof and/or sequestering carbon dioxide by growing the plants are also provided.

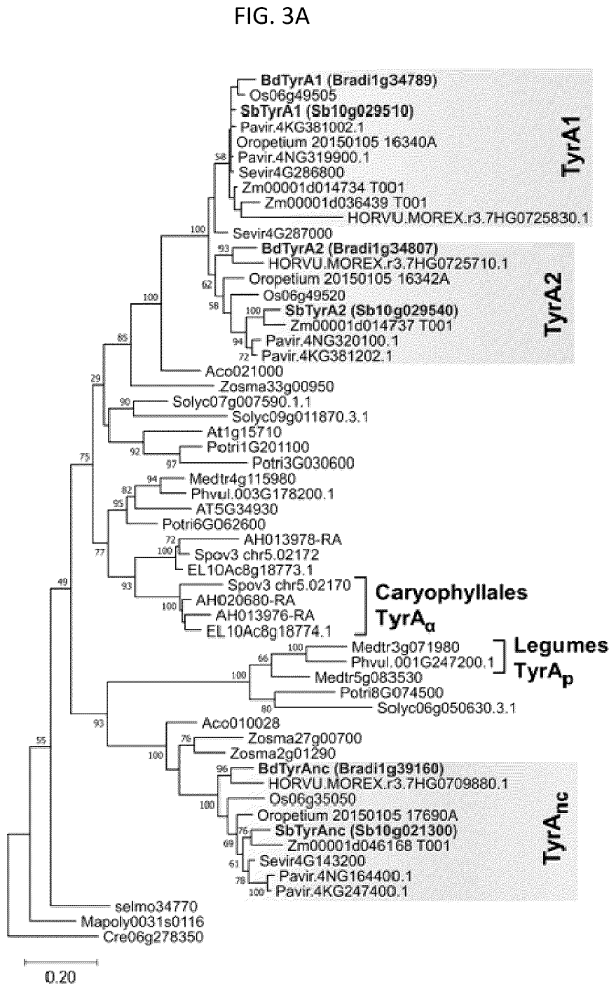

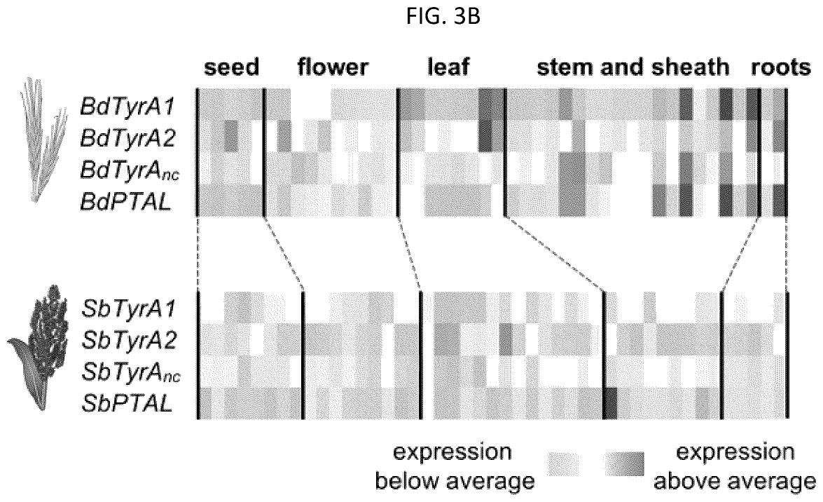

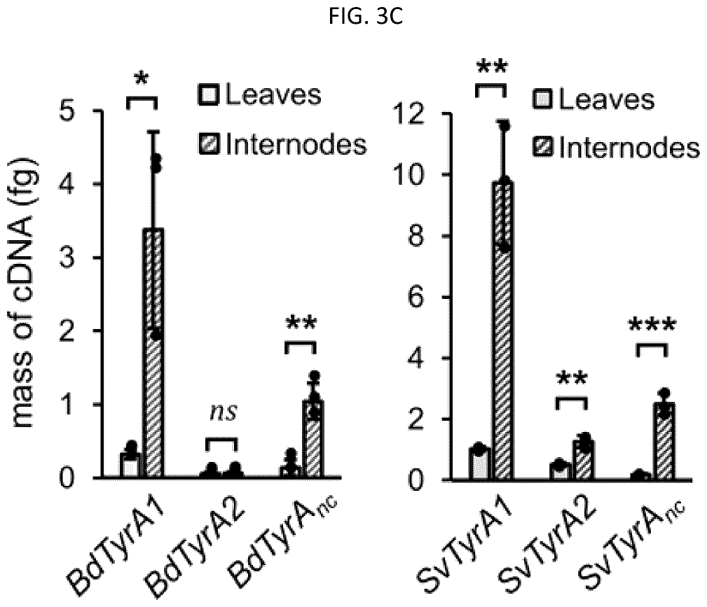

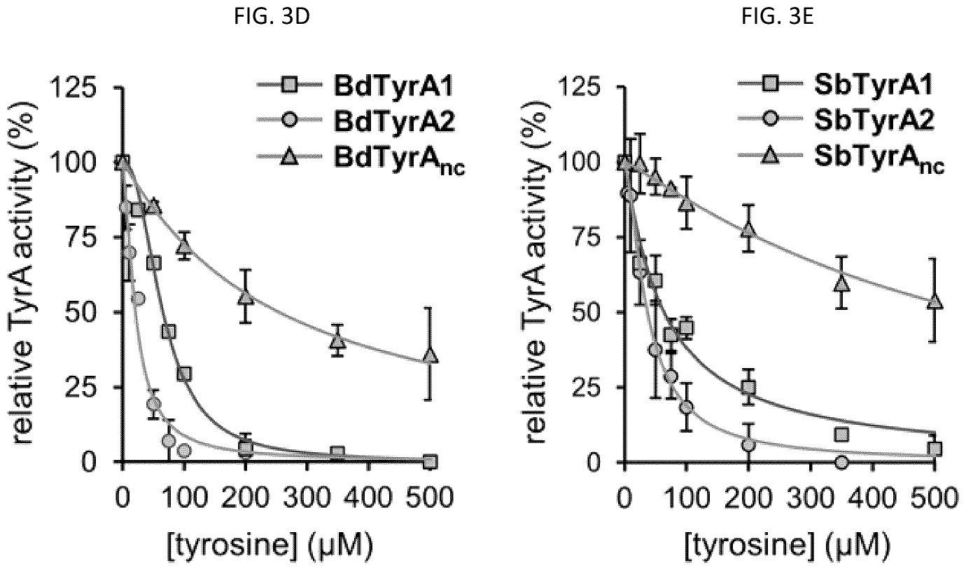

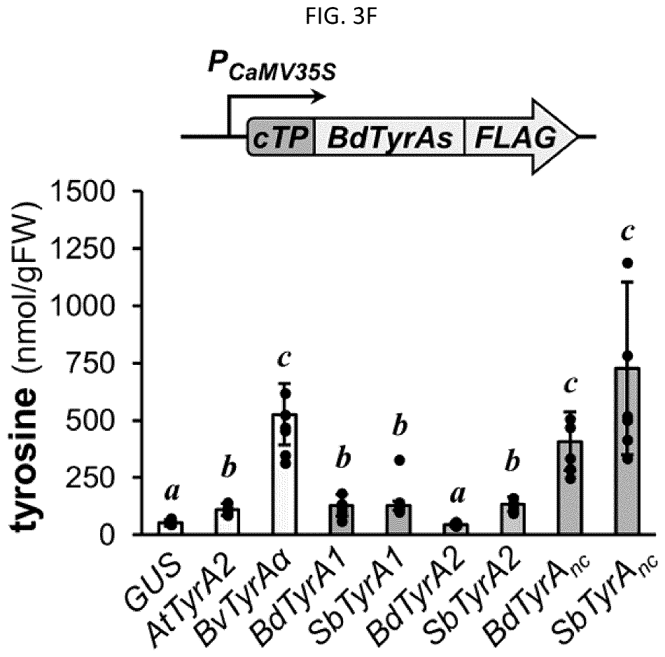

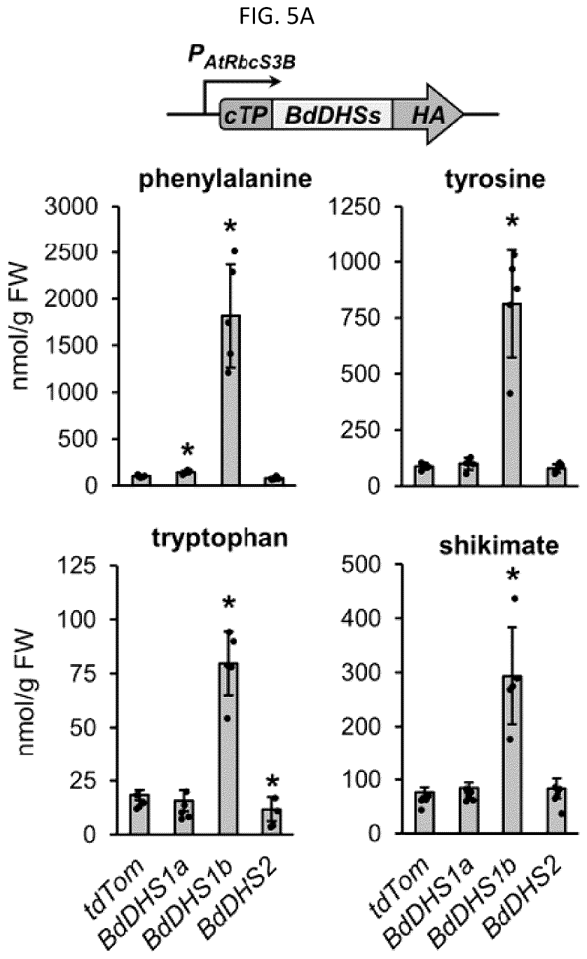

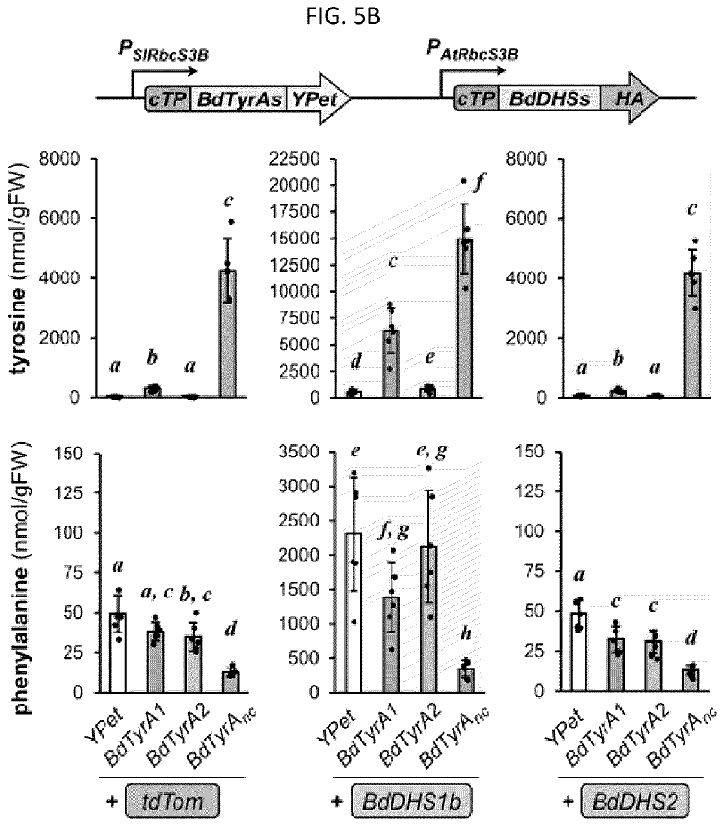

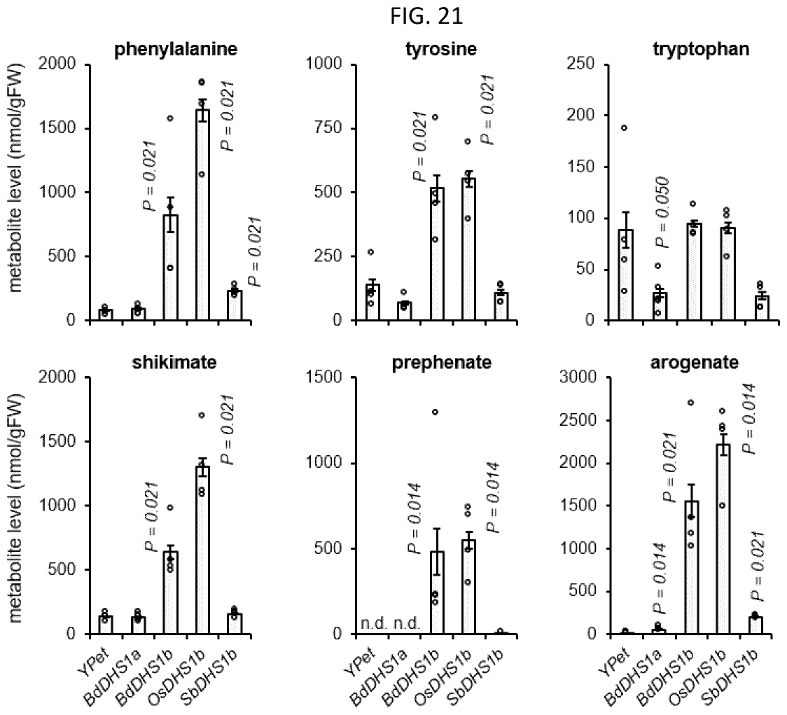

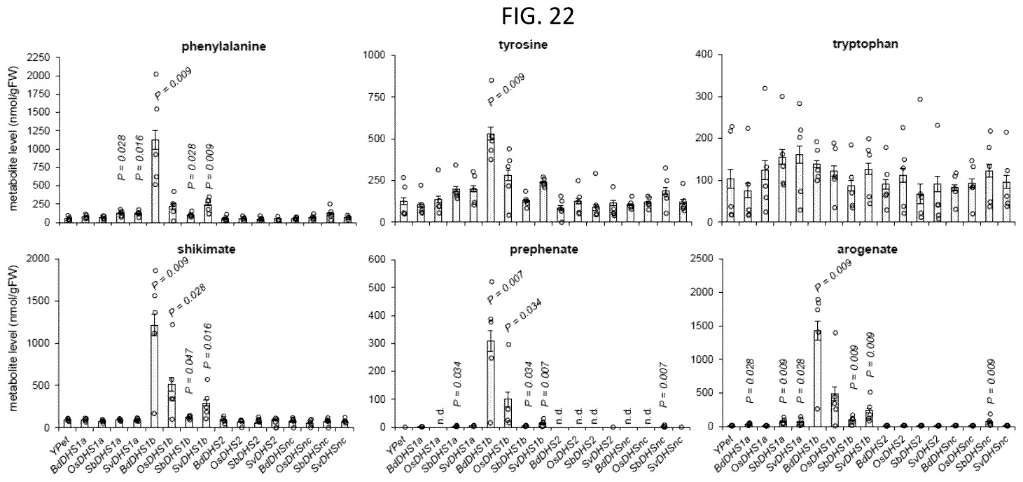

As is described in the Examples, the present inventors have identified 3-deoxy-D-arabino-heptulosonate 7-phosphate synthase (DHS) and TyrA arogenate dehydrogenase (TyrA) enzymes in grasses that are naturally “deregulated,” i.e., exhibit low sensitivity to feedback inhibition. They demonstrate that transiently expressing these deregulated enzymes in a non-grass plant (i.e., Nicotiana benthamiana) results in increased production of aromatic amino acids. Specifically, they show that expression of a DHS1b enzyme from Brachypodium distachyon (BdDHS1b; SEQ ID NO: 3), Oryza sativa (OsDHS1b; SEQ ID NO: 19), or Setaria viridis (SvDHS1b; SEQ ID NO: 27) increases the production of aromatic amino acids to varying degrees in Nicotiana benthamiana leaves (FIG. 5A, FIG. 21, FIG. 22), and that expression of a nonconical TyrA enzyme from Brachypodium distachyon (BdTyrAnc; SEQ ID NO: 37) or Sorghum bicolor (SbTyrAnc; SEQ ID NO: 43) increases the production of tyrosine in Nicotiana benthamiana leaves (FIG. 3F).

Expression of the deregulated enzymes described herein can be used to increase the production and accumulation of aromatic amino acids and their derivatives in plants. These products are valuable, and many are difficult to synthesize. Thus, increasing the levels of these products in plants being grown for pulp, paper, or biofuel production increases the value of the waste left following biomass extraction. Moreover, expression of the deregulated enzymes may enhance the ability of plants to pull carbon from the atmosphere to feed into the aromatic amino acid biosynthesis pathway and downstream pathways, resulting in increased carbon flow from carbon dioxide (CO₂) into diverse plant products, including phenylpropanoid compounds.

### Engineered Cells:

In a first aspect, the present invention provides cells engineered to express or overexpress a deregulated enzyme selected from: (a) a DHS1b enzyme comprising SEQ ID NO: 3 (BdDHS1b), SEQ ID NO: 19 (OsDHS1b), SEQ ID NO: 27 (SvDHS1b) or a DHS1b enzyme having at least 95% identity to one of SEQ ID NOs: 3, 19 or 27; or (b) a noncanonical TyrA enzyme comprising SEQ ID NO: 37 (BdTyrAnc), SEQ ID NO: 43 (SbTyrAnc) or a TyrA enzyme having at least 95% identity to SEQ ID NO: 37 or 43. Cells that express or overexpress combinations of two or more of these enzymes are also provided and may also be used herein.

A “cell” is a mass of cytoplasm that is bound externally by a cell membrane. This term encompasses both isolated single cells and cells that exists in cellular aggregates. The cells of the present invention may be from any organism and may be of any cell type. For example, the cells may be bacterial cells, fungal cells, archaeal cells, animal calls, or plant cells. However, in preferred embodiments, the cells are plant cells. Examples of suitable plant cells for use with the present invention include, without limitation, tomato plant cells, tobacco plant cells, soybean plant cells, cotton plant cells, poplar plant cells, sorghum plant cells, rice plant cells, corn plant cells, beet plant cells, mung bean plant cells, opium poppy plant cells, alfalfa plant cells, wheat plant cells, barley plant cells, millet plant cells, oat plant cells, rye plant cells, rapeseed plant cells, miscanthus plant cells, and grass plant cells.

The term “enzyme” is used to describe a biological catalyst (i.e., a substance that speeds up a chemical reaction). The enzymes of the present invention are proteins. The terms “protein,” “polypeptide,” and “peptide” are used interchangeably herein to refer to a series of amino acid residues connected by peptide bonds between the alpha-amino and carboxy groups of adjacent residues. Proteins may include modified amino acids and amino acid analogs. “Percentage of sequence similarity” or “percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Karlin and Altschul, 1990, Proc. Natl. Acad. Sci. USA 87:2267-2268; Altschul et al., 1997, Nucl. Acids Res. 25:3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. The BLAST programs can be used with the default parameters or with modified parameters provided by the user. The term “substantial identity” of amino acid sequences for purposes of this invention normally means polypeptide sequence identity of at least 80%. Preferred percent identity of polypeptides can be any integer from 80% to 100%. Allelic differences in proteins are encompassed herein and thus the sequences provided include sequences with at least 80%, 85%, 86%, 87%, 88%, 89%, 90%, 91%, 92%, 93%, 94%, 95%, 96%, 97%, 98%, 99%, or 100% identity to those sequences provided herein.

The grass enzymes described herein are “deregulated” in that they exhibit reduced sensitivity to feedback inhibition by one or more effectors as compared to other isoforms of the enzyme found in the same grass plant and/or as compared to homologs of the enzyme found in non-grass plants. As used herein, the term “effector” refers to an organic molecule, other than the substrate of the reaction catalyzed by an enzyme, that can physically interact with the enzyme and interfere with its activity. For example, the inventors have demonstrated that the noncanonical TyrA enzymes from Brachypodium distachyon (BdTyrAnc; SEQ ID NO: 37) and Sorghum bicolor (SbTyrAnc; SEQ ID NO: 43) exhibit a lower sensitivity to inhibition by tyrosine as compared to the TyrA1 and TyrA2 isoforms found in the same plants, such that they remain active at concentrations of tyrosine that completely inactivate these other isoforms (FIG. 3D and FIG. 3E).

The cells of the present invention are “engineered,” meaning that they have been altered by the hand of man. Specifically, the cells have been engineered to either express or overexpress a deregulated DHS or TyrA enzyme. In some embodiments, the cell is engineered to express an enzyme that is not native to the cell. In other embodiments, the cell is engineered to overexpress an enzyme that is native to the cell as compared to a control cell. As used herein, the term “native” is used to describe an enzyme that is naturally expressed by a cell. Conversely, an enzyme that is “not native” to a cell is an enzyme that is not naturally expressed by the cell.

A cell “overexpresses” an enzyme if it is artificially forced to express the enzyme at a higher level than the enzyme is expressed in a control cell or at a higher level than the enzyme would be expressed naturally in the absence of the genetic engineering or recombinant expression of the enzyme. As used herein, a “control cell” is a comparable cell (e.g., of the same species, cell type, and age) that developed under the same or substantially similar conditions but that was not engineered to express or overexpress a deregulated enzyme described herein.

In some embodiments, the cell is engineered to express or overexpress the deregulated enzyme via introduction of an exogenous nucleic acid (i.e., a nucleic acid that is not native to the cell) encoding the enzyme. The exogenous nucleic acid may either be inserted in the genome of the cell or may be present extrachromosomally (i.e., outside of the cell's chromosomes).

The terms “nucleic acid,” “polynucleotide,” and “oligonucleotide” are used interchangeably to refer a polymer of DNA or RNA. A nucleic acid may be single-stranded or double-stranded and may represent the sense or the antisense strand. A nucleic acid may be synthesized or obtained from a natural source. A nucleic acid may contain natural, non-natural, or altered nucleotides, as well as natural, non-natural, or altered internucleotide linkages (e.g., phosphoroamidate linkages, phosphorothioate linkages). The term nucleic acid encompasses any form of DNA or RNA including, without limitation, constructs, vectors, plasmids, messenger RNA (mRNA), and viral RNA. Those of skill in the art understand the degeneracy of the genetic code and that a variety of nucleic acids can encode the same polypeptide. Examples of suitable nucleic acid sequences encoding deregulated enzymes described herein include SEQ ID NO: 4, which encodes the BdDHS1b enzyme of SEQ ID NO: 3; SEQ ID NO: 20, which encodes the OsDHS1b enzyme of SEQ ID NO: 19; SEQ ID NO: 28, which encodes the SvDHS1b enzyme of SEQ ID NO: 27; SEQ ID NO: 38, which encodes the BdTyrAnc enzyme of SEQ ID NO: 37; and SEQ ID NO: 44, which encodes the SbTyrAnc enzyme of SEQ ID NO: 43.

In some embodiments, the exogenous nucleic acid further comprises a promoter operably linked to the nucleic acid encoding the enzyme. As used herein, the term “promoter” refers to a DNA sequence that defines where transcription of a nucleic acid begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a nucleic acid if the promoter is positioned such that it can affect transcription of the nucleic acid.

The promoter used in the nucleic acids described herein may be a heterologous promoter (i.e., a promoter that is not naturally associated with the native gene encoding deregulated enzyme), an endogenous promoter (i.e., a promoter that is naturally associated with the native gene encoding deregulated enzyme), or a synthetic promoter that is designed to function in a desired manner in a particular host cell. Suitable promoters for use with the present invention include, but are not limited to, constitutive, inducible, temporally regulated, developmentally regulated, chemically regulated, tissue-preferred, and tissue-specific promoters. In some cases, it may be advantageous to use a tissue-specific promoter or a developmental stage-specific promoter to drive expression of the deregulated enzyme in a particular tissue of an organism or during a particular developmental stage. For example, one may wish to drive expression of the deregulated enzyme in a plant tissue in which lignin deposition takes place, such as in growing stems.

In preferred embodiments, the cell is a plant cell and the promoter is a “plant promoter,” i.e., a promoter that is active in plant cells. Suitable plant promoters include, without limitation, the 35S promoter of the cauliflower mosaic virus, the tCUP cryptic constitutive promoter, the Rsyn7 promoter, the maize In2-2 promoter, the maize ubiquitin promoter, the tobacco PR-1a promoter, the Arabidopsis RuBisCO S3B promoter, and the Arabidopsis ubiquitin ligase promoter.

In the Examples, the inventors demonstrate that expressing different combinations of grass DHS and TyrA enzymes in Nicotiana benthamiana leaves results in production of different levels of the aromatic amino acids tyrosine and phenylalanine. Specifically, they demonstrate that co-expressing the DHS1b enzyme from Brachypodium distachyon (BdDHS1b; SEQ ID NO: 3) with the nonconical TyrA enzymes from Brachypodium distachyon (BdTyrAnc; SEQ ID NO: 37) increases tyrosine production in Nicotiana benthamiana leaves by 500-fold and phenylalanine production by 8-fold compared to control leaves (i.e., Nicotiana benthamiana leaves that do not express non-native DHS and TyrA enzymes), whereas co-expressing BdDHS1b with the feedback-regulated TyrA1 enzyme from Brachypodium distachyon (BdTyrA1; SEQ ID NO: 33) increases tyrosine production by 230-fold and phenylalanine production by 32-fold compared to control leaves (FIG. 5B). These data demonstrate that expression of different combinations of grass DHS and TyrA enzymes can be utilized to shift the flow through the aromatic amino acid synthesis pathway in different ways. Thus, in some embodiments, the cell is engineered to express or overexpress both a deregulated DHS1b enzyme and a specific TyrA enzyme. The TyrA enzyme may be any one of the three TyrA isoforms found in grasses, which are referred to herein as TyrA1, TyrA2, and non-canonical TyrA (TyrAnc). Specifically, the TyrA enzyme may be selected from any one of the TyrA enzymes tested in the Examples, namely SEQ ID NO: 33 (BdTyrA1), SEQ ID NO: 35 (BdTyrA2), SEQ ID NO: 37 (BdTyrAnc), SEQ ID NO: 39 (SbTyrA1), SEQ ID NO: 41 (SbTyrA2), and SEQ ID NO: 43 (SbTyrAnc). Notably, TyrA enzymes having at least 95% or more sequence identity to those sequences provided herein may also be used.

The cells of the present invention may be further engineered to express one or more additional aromatic amino acid/phenylpropanoid biosynthesis pathway enzymes. Examples of such enzymes include the prephenate and arogenate TyrA dehydrogenases and engineered PAL enzymes described in U.S. Patent Publication US2015/0150157, U.S. Patent Publication US2018/0265880, U.S. Patent Publication US2018/0216083, and U.S. patent application Ser. No. 18/611,181, and the engineered DHS enzymes described in International Patent Application Publication No. WO2023108018, the contents of which are each incorporated by reference in their entireties.

### Plants:

In a second aspect, the present invention provides plants comprising the engineered cells described herein. The term “plant” is used broadly herein to refer to a plant at any stage of development or to part of a plant, including a plant cutting, a plant cell culture, a plant organ, a plant tissue, a plant seed, or a plantlet. Particularly useful parts of a plant include harvestable parts and parts that can be used for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, cells, callus and the like.

The plants of the present invention may comprise a single engineered plant cell, comprise a plurality of engineered plant cells, or consist entirely of engineered plant cells. For example, the plants may comprise a specific organ or tissue that comprises engineered plant cells.

In some embodiments, the plant is a tomato plant, tobacco plant, soybean plant, cotton plant, poplar plant, sorghum plant, corn plant, beet plant, mung bean plant, opium poppy plant, alfalfa plant, wheat plant, barley plant, millet plant, oat plant, rye plant, rapeseed plant, miscanthus plant, or grass plant. In some embodiments, the deregulated enzyme expressed by the plant cell is native to the plant. In these embodiments, the plant may be a grass plant selected from a Sorghum bicolor plant, an Oryza sativa plant, and a Brachypodium distachyon plant.

In the Examples, the inventors demonstrate that engineering plants to express the deregulated DHS and TyrA enzymes described herein increases their production of one or more aromatic amino acids. Thus, in some embodiments, the quantity of aromatic amino acids (i.e., tyrosine, phenylalanine, and/or tryptophan) or derivatives thereof produced by the plant is greater than the quantity produced by a control plant. Increased plant production of aromatic amino acids results in increased accumulation of aromatic amino acids in plant tissues, and the terms “increased production” and “increased accumulation” are therefore used interchangeably herein. Thus, in some embodiments, the quantity of aromatic amino acids or derivatives thereof accumulated in the plant is greater than the quantity accumulated in a control plant. Suitably, the plant produces/accumulates at least one aromatic amino acid in a quantity that is at least 1.5-, 2-, 3-, 4-, 5-, 6-, 7-, 8-, 9-, 10-, 11-, 12-, 13-, 14-, 15-, 16-, 17-, 18-, 19-, or 20-fold higher as compared to the quantity produced/accumulated by a control plant. The plant may exhibit increased accumulation of these products in any tissue. Namely, the plant may exhibit increased accumulation of these products in either a tissue that comprises one or more engineered cells described herein or a tissue that does not comprise engineered cells (i.e., via export of products from the engineered cells). Production/accumulation of aromatic amino acids may be measured, for example, using stable isotope tracing (e.g., ¹³CO₂ labeling) followed by quantification via gas chromatography-mass spectrometry (GC-MS), liquid chromatography-mass spectrometry (LC-MS), or nuclear magnetic resonance (NMR).

As used herein, the term “control plant” refers to a comparable plant (e.g., of the same species, cultivar, and age) that was raised under the same or substantially similar conditions but that does not comprise an engineered cell described herein (i.e., a cell that expresses or overexpresses a deregulated enzyme selected from BdDHS1b, OsDHS1b, SvDHS1b, BdTyrAnc, and SbTyrAnc). Plants that are grown in “substantially similar conditions” are grown in similar locations and soil conditions, are planted with similar timing, are subjected to similar abiotic stresses, and the like.

In some embodiments, the plant assimilates a greater quantity of CO₂ or assimilates CO₂ at a greater rate as compared to a control plant. Suitably, the phenylpropanoid compound or CO₂ assimilation of the plant is at least 2%, 5%, 10%, 20%, 30%, 40%, 50%, or 60% greater than that of a control plant. CO₂ assimilation may be quantified by measuring the gas exchange activity of the plant. For example, CO₂ assimilation may be measured using an LI-6400XT photosynthesis system equipped with the 6400-40 leaf chamber (LI-COR). Alternatively, labeled ¹³CO₂ can be fed to plants and the rate of ¹³C incorporation into plants can be measured over time.

### Methods for Increasing Production of Aromatic Amino Acids and Derivatives Thereof:

In a third aspect, the present invention provides methods for increasing production of one or more aromatic amino acid or derivative thereof in a cell. The methods comprise engineering the cell to express or overexpress an enzyme selected from: (a) a DHS1b enzyme comprising SEQ ID NO: 3 (BdDHS1b), SEQ ID NO: 19 (OsDHS1b), SEQ ID NO: 27 (SvDHS1b) or sequences having at least 95% identity thereto; or (b) a noncanonical TyrA enzyme comprising SEQ ID NO: 37 (BdTyrAnc), SEQ ID NO: 43 (SbTyrAnc) or sequences having at least 95% sequence identity thereto. In some embodiments, the cell is engineered to express an enzyme that is not native to the cell. In other embodiments, the cell is engineered to overexpress an enzyme that is native to the cell as compared to a control cell. In preferred embodiments, the cell is a plant cell.

In some embodiments, the cell is engineered by introducing an exogenous nucleic acid encoding the enzyme into the cell. Suitable methods for introducing nucleic acids into cells include, without limitation, Agrobacterium-mediated transformation, the floral dip method, bacteriophage or viral infection, electroporation, heat shock, lipofection, microinjection, and particle bombardment. In these embodiments, the exogenous nucleic acid may either be inserted into the genome of the cell or remain extrachromosomal after it has been introduced into the cell. Insertion into the genome may be random or targeted to a specific locus (e.g., via homologous recombination). Further, in these embodiments, the exogenous nucleic acid may comprise a promoter operably linked to the nucleic acid encoding the enzyme, as described above.

In some embodiments, the cell is engineered via genome editing. In cells in which the deregulated enzyme is not native, the genome may be engineered to insert a copy of a DNA sequence encoding the deregulated enzyme or to replace a sequence encoding a regulated homolog of the deregulated enzyme with a sequence encoding the deregulated enzyme. In cells in which the deregulated enzyme is native, the genome may be engineered to introduce one or more additional copies of a DNA sequence encoding the deregulated enzyme or to modify a gene regulatory element (e.g., a promoter, an enhancer) associated with the native gene encoding the deregulated enzyme to increase its expression. Genome editing may involve use of an engineered nuclease (e.g., a meganuclease, zinc finger nuclease (ZFN), transcription activator-like effector nuclease (TALEN), or CRISPR-Cas nuclease).

As is described above, the inventors have demonstrated that expressing different combinations of grass DHS and TyrA enzymes in Nicotiana benthamiana leaves results in production of different levels of the aromatic amino acids tyrosine and phenylalanine. Thus, in some embodiments, the cell is engineered to express or overexpress a specific combination of a deregulated DHS1b enzyme and a TyrA enzyme. The TyrA enzyme may be any one of the three TyrA isoforms found in grasses (i.e., TyrA1, TyrA2, and TyrAnc).

In some embodiments, the cell is part of a plant, and the methods comprise engineering one or more cells of the plant to express or overexpress the deregulated enzyme. In these embodiments, a single cell, a plurality of cells (e.g., a specific organ or tissue), or all the cells of the plant may be engineered.

In some embodiments, the methods further comprise purifying aromatic amino acids or derivatives thereof from the cell. As used herein, the term “purifying” refers to the process of separating a desired product from other cellular components and impurities. Suitable methods for purifying aromatic amino acids and derivatives thereof include, without limitation, high performance liquid chromatography (HPLC) and other chromatographic techniques, such as affinity chromatography. A “purified” product may be at least 85% pure, at least 95% pure, or at least 99% pure.

### Methods for Using Plants:

In a fourth aspect, the present invention provides methods for using the plants described herein to (1) produce aromatic amino acids or derivatives thereof, or (2) sequester CO₂. Both sets of methods comprise growing the plants described herein. The methods for producing aromatic amino acids or derivatives thereof further comprise purifying the aromatic amino acids or derivatives thereof produced by the plant.

As used herein, a “derivative” of an aromatic amino acid is any cellular product that is produced using an aromatic amino acid or that incorporates an aromatic amino acid. Examples of aromatic amino acid derivatives that could be produced using the methods of the present invention include the tyrosine derivatives homogentisate (HGA), α-tocopherols, and γ-tocopherols. Suitable products made from overexpression of the enzymes as described herein may include, without limitation, vitamin E, plastoquinone, a cyanogenic glycoside, a benzylisoquinoline alkaloid, rosmarinic acid, betalains, suberin, mescaline, morphine, salidroside, a phenylpropanoid compound, dhurrin, a tocochromanol, ubiquinone, lignin, a catecholamine such as epinephrine (adrenaline) or dopamine (i.e., L-dihydroxyphenylalanine (L-DOPA)), melanin, an isoquinoline alkaloid, hydroxycinnamic acid amide (HCAA), an amaryllidaceae alkaloid, hordenine, hydroxycinnamate, hydroxylstyrene, phenylethanol, phenyllactate, phenylacetic acid, mandelic acid, or tyrosine. Phenylpropanoid compounds (e.g., lignin, tannins, flavonoids, stilbene, resveratrol, lignans) may be produced from tyrosine.

“Carbon sequestration” is a process in which atmospheric CO₂ is captured and stored. It is one method for reducing the amount of CO₂ in the atmosphere (i.e., to reduce global climate change). In some embodiments, the methods further comprise harvesting part of the plant while leaving the roots of the plant in the soil such that the carbon contained in the roots is sequestered therein. Harvestable parts of plants include, without limitation, flowers, pollen, seedlings, tubers, leaves, stems, fruit, seeds, roots, cuttings, and the like. Above ground tissues that are enriched for aromatic compounds will be decomposed slowly by soil microbes, which also enhances carbon sequestration. The harvested plant materials can be also converted via pyrolysis to biochar, which can substantially extend the retention of organic molecules and carbon sequestration.

### Enzyme Sequences:

TABLE 4
Grass DHS enzyme sequences

| | | Protein | DNA (CDS) |
|---|---|---|---|
| Enzyme | Organism | sequence | sequence |
| BdDHS1a | Brachypodium distachyon | SEQ ID NO: 1 | SEQ ID NO: 2 |
| BdDHS1b | Brachypodium distachyon | SEQ ID NO: 3 | SEQ ID NO: 4 |
| BdDHS2 | Brachypodium distachyon | SEQ ID NO: 5 | SEQ ID NO: 6 |
| BdDHSnc | Brachypodium distachyon | SEQ ID NO: 7 | SEQ ID NO: 8 |
| SbDHS1a | Sorghum bicolor | SEQ ID NO: 9 | SEQ ID NO: 10 |
| SbDHS1b | Sorghum bicolor | SEQ ID NO: 11 | SEQ ID NO: 12 |
| SbDHS2 | Sorghum bicolor | SEQ ID NO: 13 | SEQ ID NO: 14 |
| SbDHSnc | Sorghum bicolor | SEQ ID NO: 15 | SEQ ID NO: 16 |
| OsDHS1a | Oryza sativa | SEQ ID NO: 17 | SEQ ID NO: 18 |
| OsDHS1b | Oryza sativa | SEQ ID NO: 19 | SEQ ID NO: 20 |
| OsDHS2 | Oryza sativa | SEQ ID NO: 21 | SEQ ID NO: 22 |
| OsDHSnc | Oryza sativa | SEQ ID NO: 23 | SEQ ID NO: 24 |
| SvDHS1a | Setaria viridis | SEQ ID NO: 25 | SEQ ID NO: 26 |
| SvDHS1b | Setaria viridis | SEQ ID NO: 27 | SEQ ID NO: 28 |
| SvDHS2 | Setaria viridis | SEQ ID NO: 29 | SEQ ID NO: 30 |
| SvDHSnc | Setaria viridis | SEQ ID NO: 31 | SEQ ID NO: 32 |

TABLE 5
Grass TyrA enzyme sequences

| | | Protein | DNA (CDS) |
|---|---|---|---|
| Enzyme | Organism | sequence | sequence |
| BdTyrA1 | Brachypodium distachyon | SEQ ID NO: 33 | SEQ ID NO: 34 |
| BdTyrA2 | Brachypodium distachyon | SEQ ID NO: 35 | SEQ ID NO: 36 |
| BdTyrAnc | Brachypodium distachyon | SEQ ID NO: 37 | SEQ ID NO: 38 |
| SbTyrA1 | Sorghum bicolor | SEQ ID NO: 39 | SEQ ID NO: 40 |
| SbTyrA2 | Sorghum bicolor | SEQ ID NO: 41 | SEQ ID NO: 42 |
| SbTyrAnc | Sorghum bicolor | SEQ ID NO: 43 | SEQ ID NO: 44 |

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

### Example 1

In the following example, the inventors describe the identification of 3-deoxy-D-arabino-heptulosonate 7-phosphate synthase (DHS) and TyrA enzymes in grasses that are naturally deregulated. They demonstrate that transiently expressing these deregulated enzymes in a non-grass plant (i.e., Nicotiana benthamiana) results in increased synthesis of aromatic amino acids. Specifically, they show that expression of a 3-deoxy-D-arabino-heptulosonate 7-phosphate synthase 1b (DHS1b) enzyme from Brachypodium distachyon (BdDHS1b; SEQ ID NO: 3) increases the production of phenylalanine, tyrosine, tryptophan, and shikimate in Nicotiana benthamiana leaves (FIG. 5A), and that expression of a nonconical TyrA enzyme from Brachypodium distachyon (BdTyrAnc; SEQ ID NO: 37) or Sorghum bicolor (SbTyrAnc; SEQ ID NO: 43) increases the production of tyrosine in Nicotiana benthamiana leaves (FIG. 3F).

### BACKGROUND

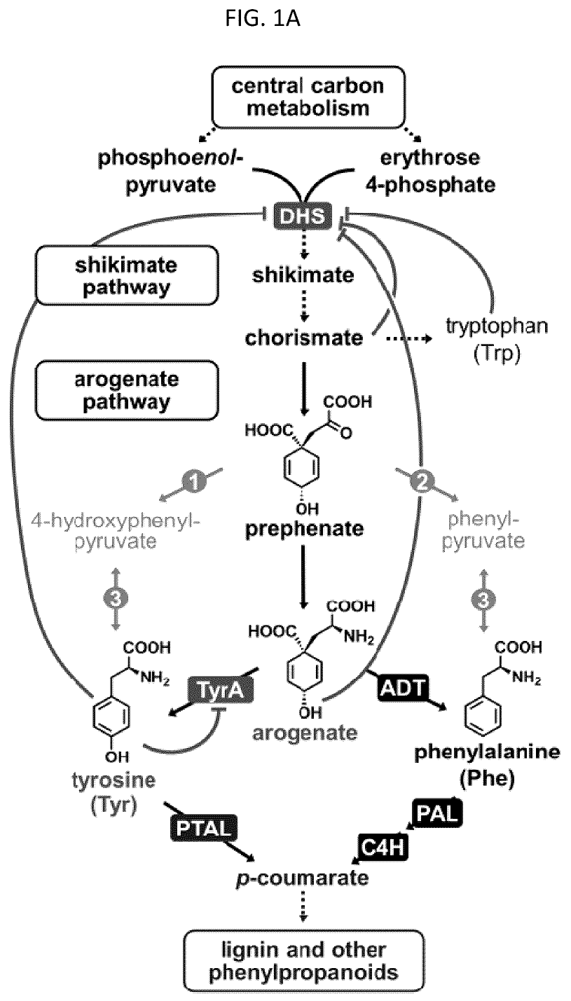

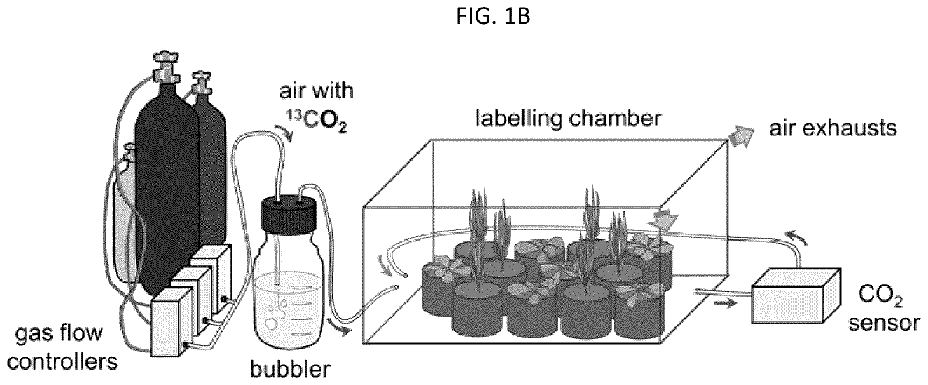

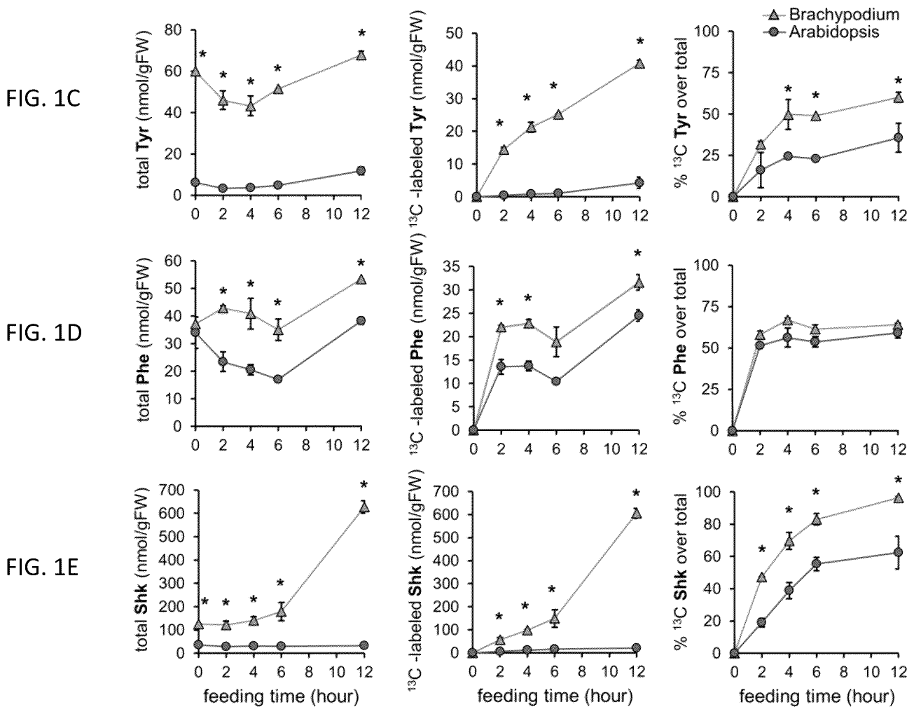

The biosynthesis of aromatic amino acids (AAAs)—phenylalanine, tyrosine, and tryptophan—represents one of the major routes of plant metabolism that supplies essential building blocks for the production of proteins and a myriad of plant natural products^{1, 2}. Yet, it remains poorly understood how the AAA biosynthetic pathway is regulated to meet various demands for AAA precursors in different species. The most abundant of these AAA derived compounds is lignin, which accounts for up to 30% of plant dry weight and plays a critical role in strengthening and waterproofing secondary cell walls. In most plant species, lignin and the other phenylpropanoids are synthesized exclusively from phenylalanine by the enzyme phenylalanine ammonia lyase (PAL, FIG. 1A)^{4, 5}. In contrast, grasses (family Poaceae), arguably one of the most important plant lineages from both an ecological and economic perspective, can produce lignin from both tyrosine and phenylalanine due to the presence of bifunctional phenylalanine/tyrosine ammonia lyase (PTAL) enzymes⁶⁻¹¹. Multiple lines of evidence support that a significant proportion of grass lignin is synthesized from tyrosine^{10, 12, 13}. However, it remains unknown how grasses regulate the upstream AAA biosynthetic pathways to provide high amount of both tyrosine and phenylalanine precursors to support the unique dual lignin pathway.

Our current knowledge on the regulation of plant AAA biosynthesis, mostly derived from dicot models, indicates that plants balance AAA production by targeting activities of key enzymes of the AAA pathway(s) through a combination of transcriptional and feedback regulation^{1,2,14}. For instance, the first enzyme in AAA biosynthesis, 3-deoxy-D-arabino-heptulosonate 7-phosphate (DAHP) synthase (DHS; EC:2.5.1.54) (FIG. 1A), is feedback regulated by AAAs and multiple downstream metabolites^{15, 16}. This feedback regulation at the entry point of the shikimate pathway controls the biosynthesis of AAAs and, when released by point mutations targeting the regulatory domain of DHS, can largely increase AAA production and CO₂ fixation in Arabidopsis thaliana¹⁶. Similarly, the enzymes controlling tyrosine and phenylalanine biosynthesis from arogenate, arogenate dehydrogenase (TyrA; EC 1.3.1.78) and arogenate dehydratase (ADT; EC 4.2.1.91), are subjected to feedback inhibition by their corresponding reaction products (FIG. 1A)^{2, 14}. However, while TyrA enzymes are sensitive to feedback inhibition even at low tyrosine levels¹⁷⁻²⁰, vascular plants have specialized ADT isoforms that maintain their activity at high concentrations of phenylalanine^{21, 22}. Furthermore, unlike bacterial DHS enzymes²³, none of the plant DHS enzymes characterized so far are inhibited by phenylalanine^{15, 24}, likely providing abundant phenylalanine precursor for phenylpropanoid production. Besides the feedback regulation at the enzyme level, DHS and ADT genes are often strongly co-expressed with PAL and other lignin and phenylpropanoid-related genes across different plants^{15, 25-29}. Through this conjunction of transcriptional and feedback regulation, many plant species prioritize the production of phenylalanine for phenylpropanoid biosynthesis, to the detriment of tyrosine and tryptophan levels^{26, 30-33}. However, given the presence of the unique dual lignin pathway, we hypothesized that grasses may regulate AAA production differently from other plants.

Here, we combine stable-isotope labeling, phylogenetic and expression analyses, detailed enzyme characterization, and combinatorial in planta expression analysis to demonstrate that the coordinated regulation of the entry and final steps of AAA biosynthesis allows grasses to efficiently provide both tyrosine and phenylalanine precursors to meet the unique demand of the dual tyrosine/phenylalanine lignin pathway. This study highlights the importance of transcriptional and biochemical regulation at key metabolic branching points in fine-tuning the supply of AAA precursors for the downstream lignin and phenylpropanoid pathway. These basic findings and the novel enzymes identified in grasses can be utilized to engineer plants to efficiently produce natural and bio-based aromatic products.

### Results:

Grasses Synthesize Tyrosine at a Much Higher Rate than Arabidopsis without Compromising Phenylalanine Production

While prior studies reported that grasses accumulate high levels of tyrosine³⁵⁻³⁷, an elevated steady-state level of a metabolite does not necessarily imply a high synthesis and usage rate³⁸. Therefore, we performed ¹³CO₂ feeding experiments to compare the turnover rates of AAAs between the grass Brachypodium distachyon Bd21-3 and the dicot Arabidopsis thaliana Col-0. Four-weeks-old Brachypodium and Arabidopsis plants, before bolting, were fed side by side (FIG. 1B) with an air mixture containing ˜400 ppm of ¹³CO₂. Then, samples were collected at regular intervals for determination of ¹³C labeled tyrosine, phenylalanine, and shikimate by ultra-high performance liquid chromatography coupled to electrospray ionization mass spectrometry (UHPLC-MS).

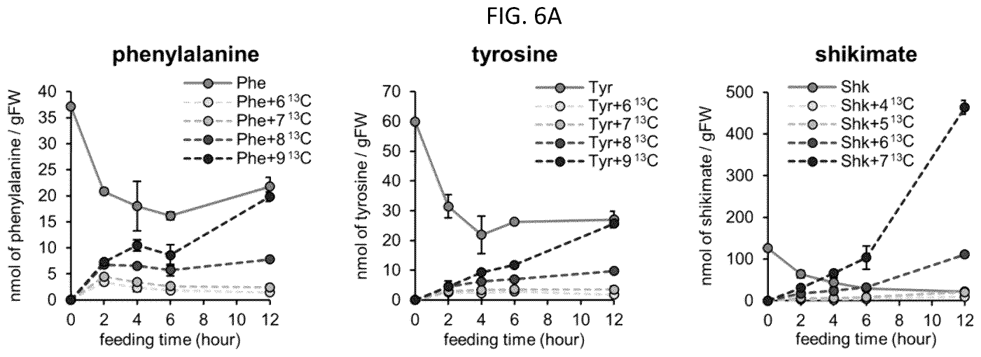

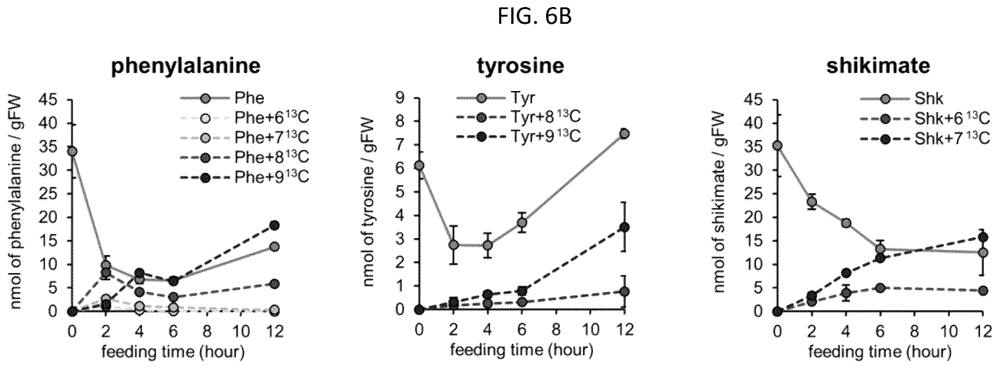

Total tyrosine content (with either ¹²C or ¹³C) ranged between ˜3 to 12 nmol per gram of fresh weight (nmol/gFW) in Arabidopsis but was much higher in Brachypodium (FIG. 1C), where it reached up to 70 nmol/gFW. Moreover, ¹³C-labeled tyrosine (mostly eight or nine ¹³C-isotopologues, FIGS. 6A-6B) was 10-times more abundant in Brachypodium (˜40 nmol/gFW) than in Arabidopsis (˜4 nmol/gFW) after 12h (FIG. 1C). In contrast, total phenylalanine levels were comparable between the two species, in the range of 35-55 nmol/gFW in Brachypodium, and 20-35 nmol/gFW, in Arabidopsis (FIG. 1D). Labeled ¹³C-phenylalanine over time was also similar between the two species (FIG. 1D). As observed for tyrosine, most ¹³C-phenylalanine was fully labeled, containing eight or nine ¹³C atoms (FIG. 6). In addition, we detected striking differences in the dynamics of the shikimate pool, with up to 20-times more total shikimate accumulating in Brachypodium than in Arabidopsis by the end of the day, and a higher incorporation rate of ¹³C (FIG. 1E).

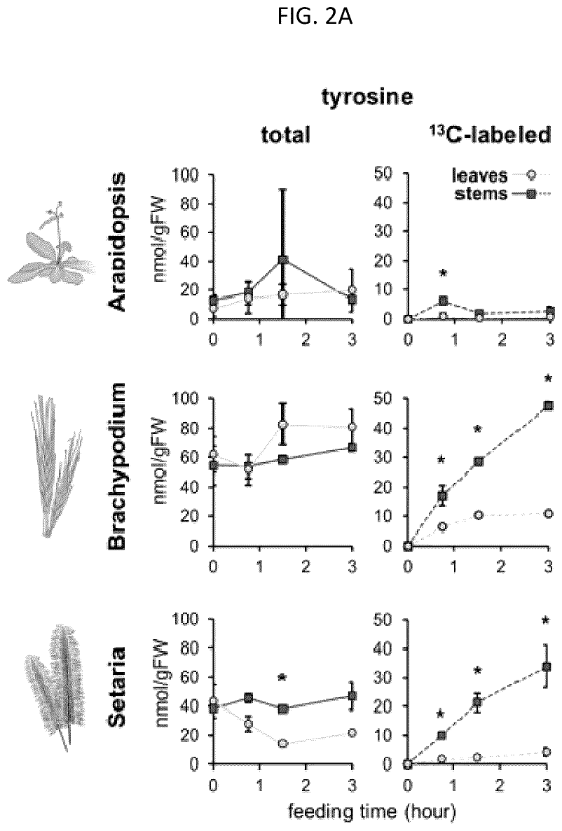

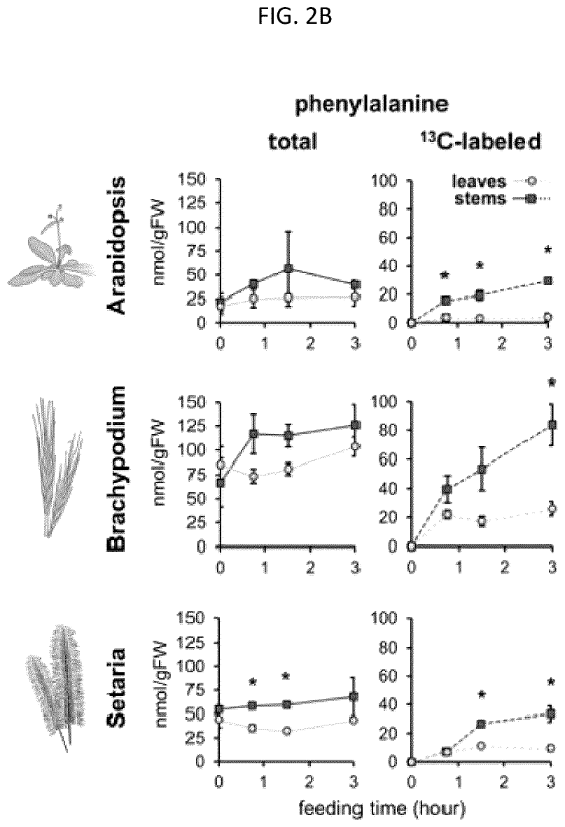

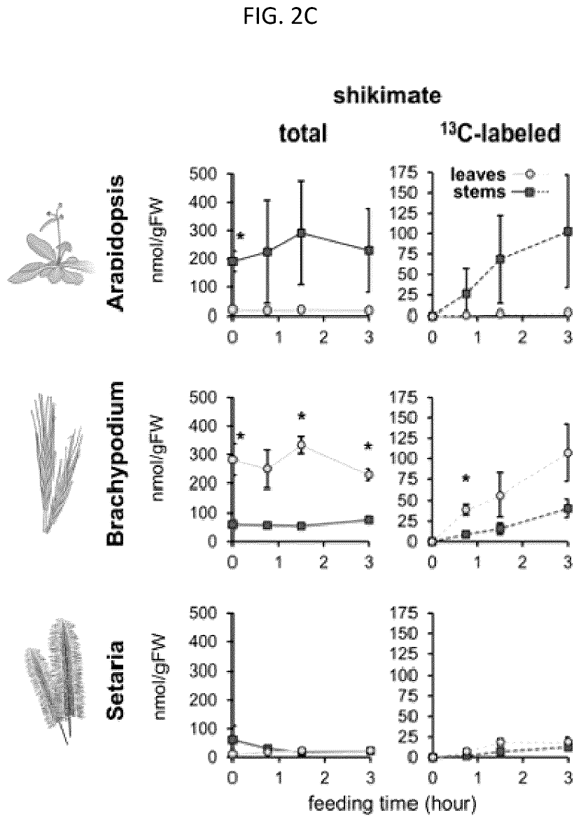

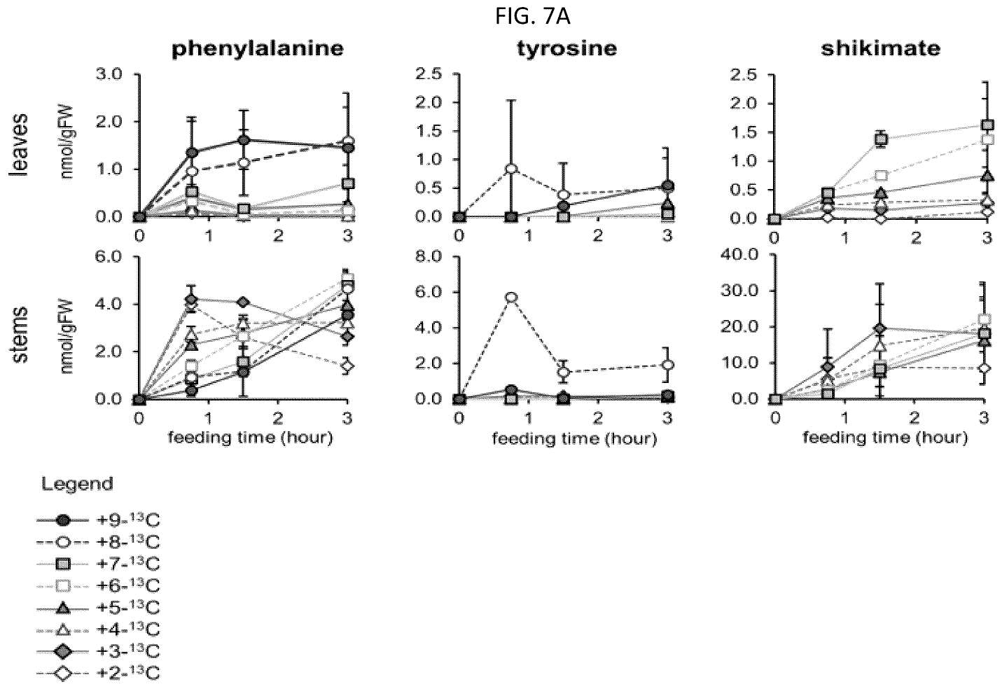

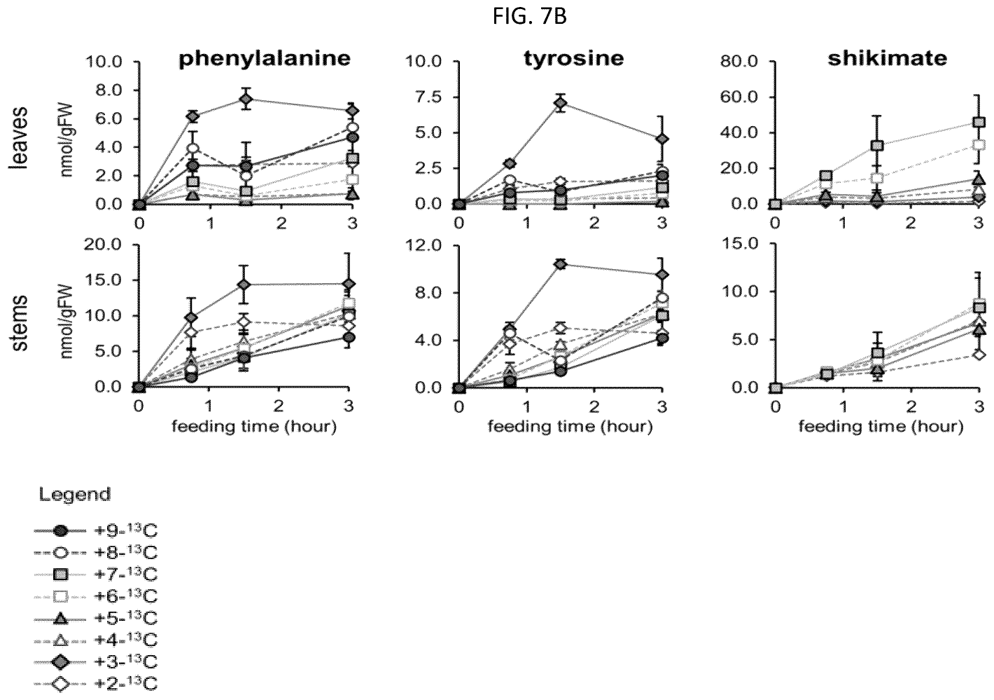

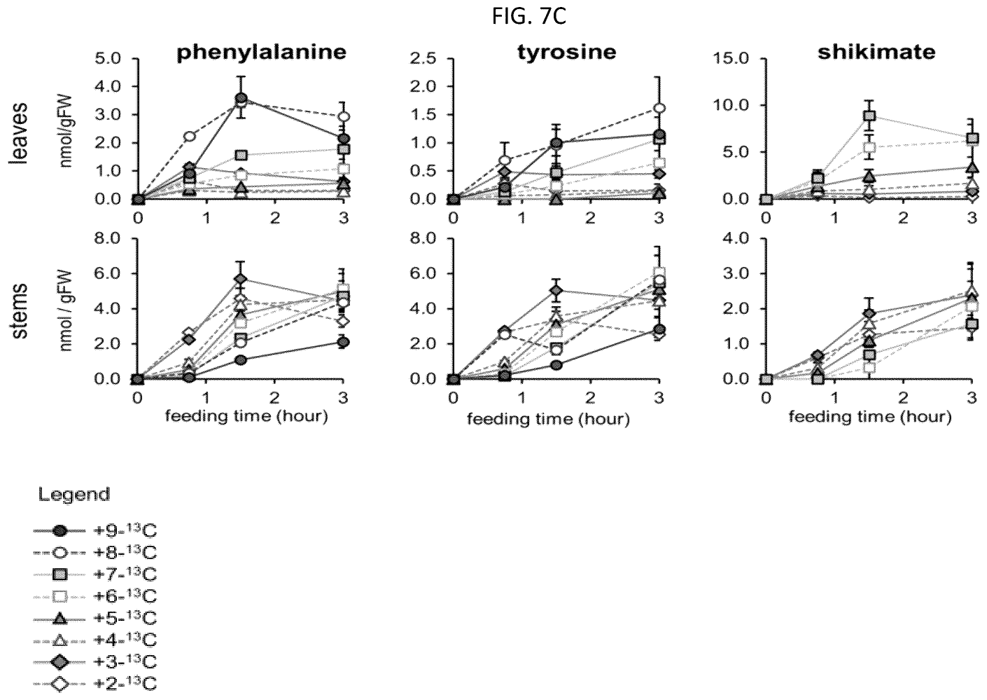

We next performed additional ¹³CO₂ labeling experiments using older six-weeks-old plants of Arabidopsis, Brachypodium, and Setaria viridis A10.1 (hereafter, Setaria), comparing young leaves with elongating stems, where lignin is actively formed. These experiments further confirmed that grass species accumulate more tyrosine than Arabidopsis (FIG. 2A) and showed that incorporation of ¹³C into tyrosine was particularly rapid in grass stems, which accumulated up to ˜50 nmol/gFW of ¹³C-tyrosine after 3 hours of ¹³CO₂ feeding (FIG. 2A), more than 10-times faster than Arabidopsis (<2 nmol/gFW; FIG. 2A). On the contrary, the three species exhibited comparable labeling kinetics for phenylalanine, with faster ¹³C-phenylalanine accumulation in the stems than in the leaves (FIG. 2B). In the case of shikimate, Arabidopsis stems showed 10 to 20-times more total shikimate and higher rate of ¹³C-labeling than the leaves (FIG. 2C), despite high biological variation. In contrast, grass species showed a faster shikimate labeling in the leaves (FIG. 2C). The time-course labeling of different ¹³C-isotopologues of phenylalanine, tyrosine, and shikimate differed between leaf and stem tissues and among species (FIG. 7). These results showed that, while the three species have a high rate of phenylalanine biosynthesis in the stems, only grasses exhibit high tyrosine turnover in this organ. Furthermore, the high rate of tyrosine biosynthesis in grasses did not seem to compromise phenylalanine biosynthesis.

### Grass TyrA1 and TyrAnc Isoforms are Highly Expressed in Growing Stems

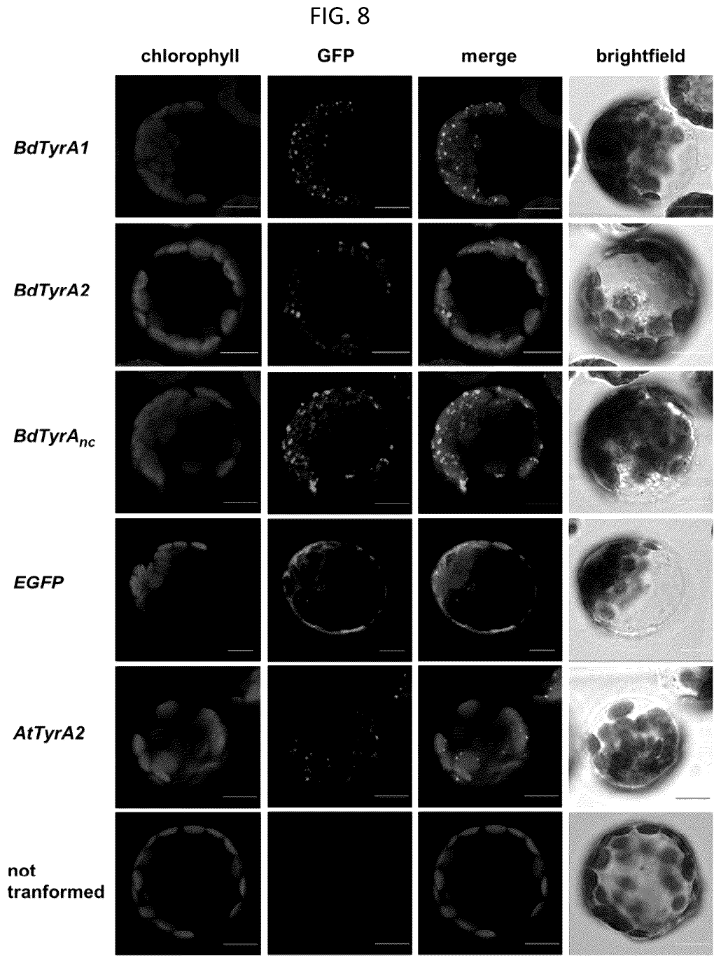

To understand the mechanism behind the increased production of tyrosine in grasses, we next examined the family of grass TyrA enzymes, which catalyze the final and key regulatory step in tyrosine biosynthesis^{2, 14, 39}. Reconstruction of the plant TyrA protein phylogeny showed that grass genomes have at least three TyrA isoforms (FIG. 3A) corresponding with the Brachypodium distachyon v3.2 loci Bradi1g34789, Bradi1g34807, and Bradi1g39160, which we named TyrA1, TyrA2, and non-canonical TyrA (TyrAnc), respectively. Whereas grass TyrA1 and TyrA2 are closely related to each other and to TyrA enzymes from most dicot plants, grass TyrAnc cluster in a more distant group and is a sister to cytosolic TyrAnc enzymes from legumes and other dicots (FIG. 3A)^{39, 40}. All three BdTyrA proteins have predicted plastid transit peptides in their N-terminus (TargetP-2.0, DTU Health Tech), similarly to most plant TyrA enzymes⁴⁰, and were targeted to the plastids when expressed in Arabidopsis protoplast fused to enhanced green fluorescent protein (EGFP) in their C-terminus (FIG. 8).

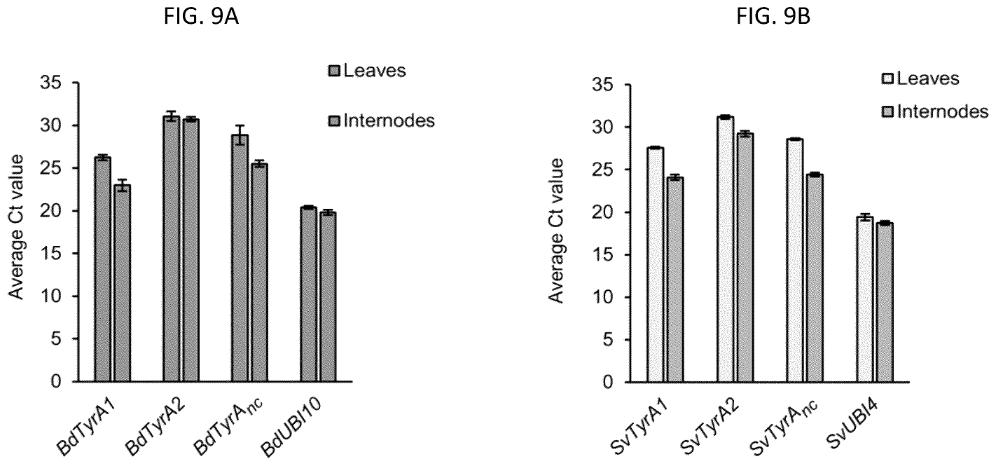

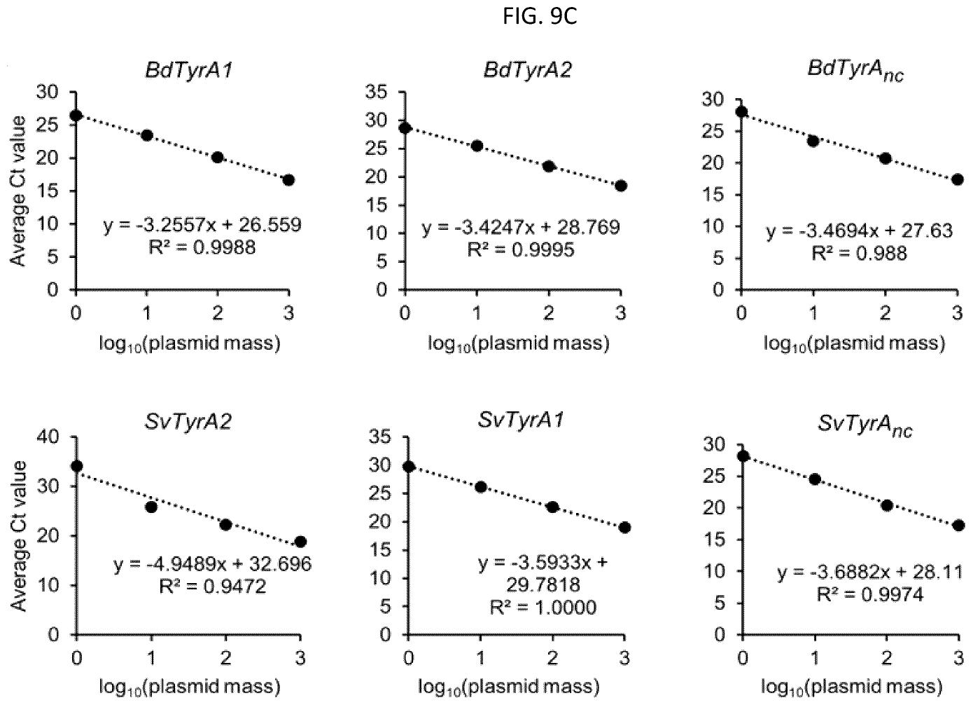

To examine the potential involvement of TyrA in the tyrosine-lignin pathway of grasses, we compared the expression of TyrA genes with PTAL using publicly available expression datasets from Brachypodium⁴¹ and Sorghum⁴². Interestingly, the expression profile of TyrA enzymes, in particular TyrA1 and TyrAnc, resembled that of PTAL, showing higher expression in stem internodes and roots, and low in seeds, flowers, and leaves (FIG. 3B), which correlates with the elevated rate of tyrosine production observed in grass internodes in the ¹³CO2 feeding experiments (FIG. 2). Furthermore, gene co-expression networks in Brachypodium⁴¹, showed that BdTyrA1 and BdPTAL (Bradi3g49250) expression correlate with many other genes of the lignin pathway (data not shown). Absolute real-time quantitative PCR (RT-qPCR) comparing TyrA expression in young leaves and developing internodes of Brachypodium and Setaria found that TyrA1 had the highest expression among TyrA genes, followed by TyrAnc and TyrA2 (FIG. 3C, FIG. 9). Importantly, TyrA1 and TyrAnc transcripts were up to 10-times more abundant in young developing internodes than in leaves (FIG. 3C). This was not clearly observed for TyrA2 genes, which showed a fold change comparable to ubiquitin ligase reference genes (˜1.5-times; FIG. 9). Altogether, these results support that the expression of TyrA1 and TyrAnc, but not TyrA2, is strongly induced in developing stems, where active PTAL expression and lignin deposition take place.

### TyrAnc Enzymes, but not TyrA1 Enzymes, Exhibit Low Sensitivity to Feedback Inhibition by Tyrosine

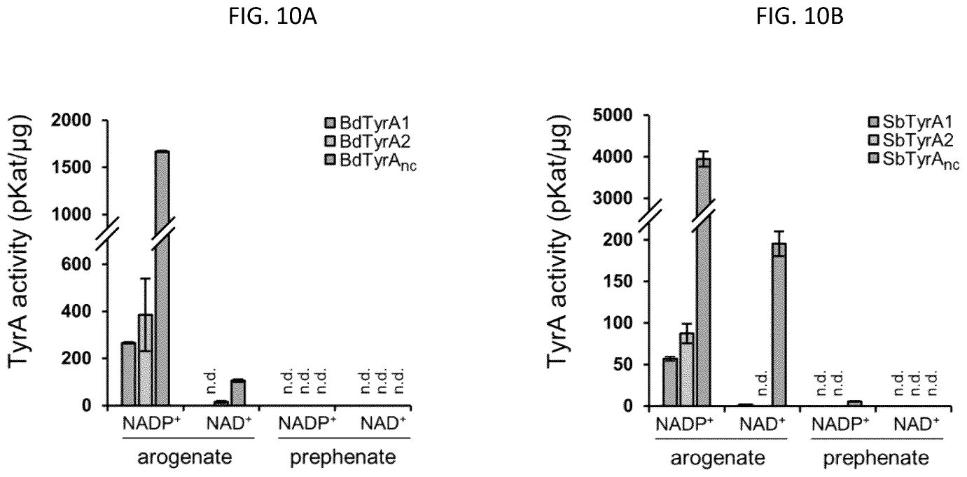

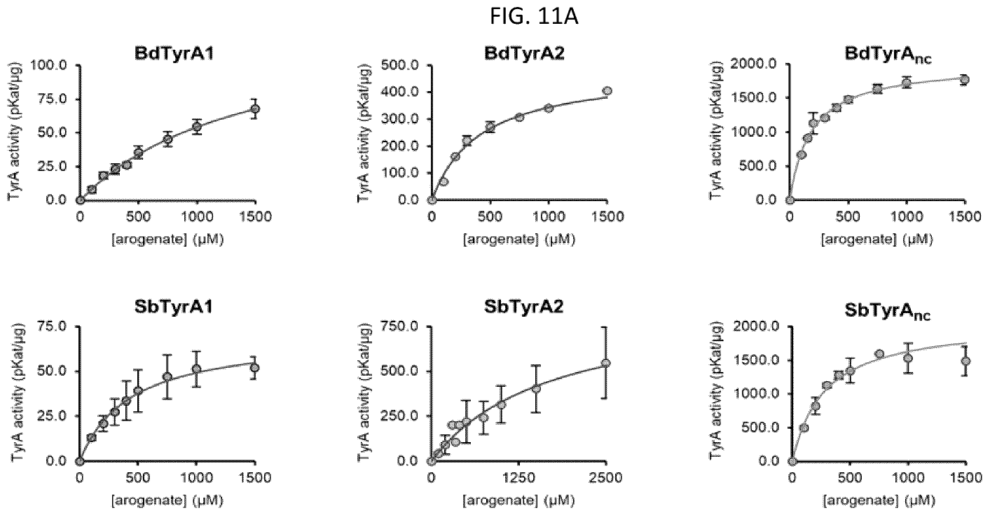

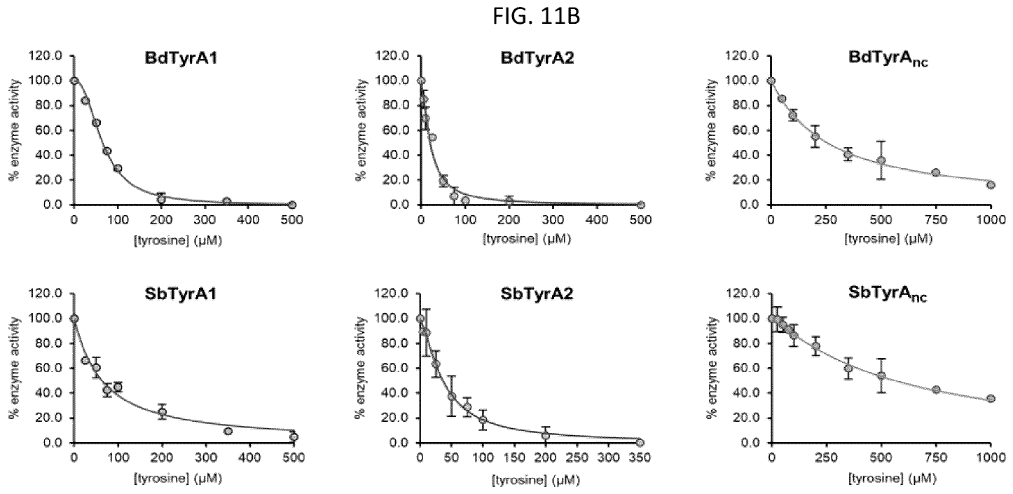

Next, to study the biochemical properties of grass TyrA enzymes, we generated and characterized recombinant purified TyrA proteins from Brachypodium (BdTyrA) and Sorghum (SbTyrA), two distantly related grass species^{43, 44}. Whereas TyrA enzymes from dicot plants are generally NADP⁺-dependent arogenate dehydrogenases^{20, 39, 45}, except for NADP⁺-prephenate dehydrogenases in the legume family^{40, 46}, previous reports on the substrate preference of grass TyrA enzymes are inconclusive^{17, 19}. Initial biochemical assays to test the substrate and cofactor preference revealed that grass TyrA enzymes are most active with arogenate as substrate, rather than prephenate, and NADP⁺ as cofactor, exhibiting only minor NAD⁺-arogenate dehydrogenase activity (up to 5% of the main activity; FIG. 10). NAD⁺-prephenate dehydrogenase activity was absent in all cases, ruling out a significant contamination of the enzyme preparations with the TyrA enzyme from E. coli^{47.48}. Detailed kinetic analyses of the activity with arogenate and NADP⁺ showed that the six TyrA enzymes obey Michaelis-Menten kinetics, with the TyrAnc isoforms having, by a wide margin, the highest turnover number (k_{cat}) and lowest Michaelis-Menten constant (Kₘ) (Table 1, FIG. 11). Consequently, the catalytic efficiencies (k_{cat}/Kₘ) of BdTyrAnc (˜580 s⁻¹ mM⁻¹) and SbTyrAnc (˜337 s⁻¹ mM⁻¹) were the highest amidst the three isoforms of each species (Table 1).

TABLE 1
Kinetic parameters of TyrA enzymes. Kₘ and k_{cat} were calculated from Michaelis-
Menten plots shown in FIG. 11. k_{cat}/Kₘ was calculated based on Kₘ, k_{cat}, and
the molecular weight of each recombinant enzyme (including the mass of the
poly-histidine tag). The half-inhibitory concentration of tyrosine (IC₅₀) was
calculated at 0.5 mM of arogenate and 1 mM of NADP⁺ from the data shown
in FIG. 11. The inhibition constant for tyrosine (Kᵢ) was calculated from Kₘ and
IC₅₀ values under a competitive inhibition model^{19, 20}. All data are means ±
SD of n = 4-6 derived from at least two independent experiments
conducted on different days using different batches of purified recombinant enzyme.

| | Kₘ | | | IC₅₀ | Kᵢ |
|---|---|---|---|---|---|
| | (mM | k_{cat} | k_{cat}/Kₘ | (μM | (μM |
| | arogenate) | (s⁻¹) | (s⁻¹ · mM⁻¹) | tyrosine) | tyrosine) |
| BdTyrA1 | 1.41 ± 0.24 | 5.2 ± 0.8 | 3.6 ± 0.8 | 66 ± 2 | 46 ± 3 |
| SbTyrA1 | 0.57 ± 0.15 | 2.9 ± 0.6 | 5.3 ± 0.8 | 71 ± 24 | 53 ± 10 |
| BdTyrA2 | 0.45 ± 0.04 | 18.5 ± 0.6 | 41.2 ± 1.7 | 20 ± 6 | 8 ± 2 |
| SbTyrA2 | 2.13 ± 0.53 | 38.5 ± 8.8 | 18.1 ± 9.6 | 47 ± 23 | 36 ± 13 |
| BdTyrAnc | 0.13 ± 0.04 | 76.5 ± 5.5 | 579.6 ± 24.3 | 242 ± 45 | 64 ± 25 |
| SbTyrAnc | 0.22 ± 0.06 | 73.5 ± 18.2 | 337.5 ± 19.5 | 406 ± 85 | 137 ± 44 |

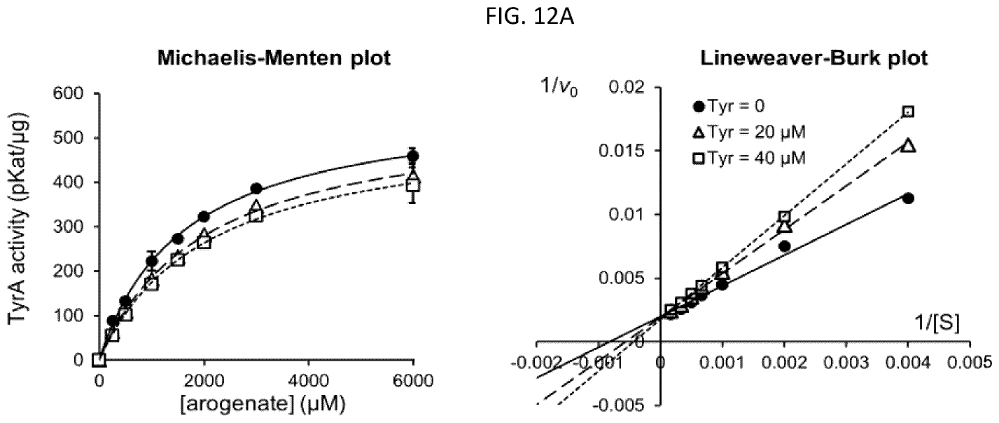

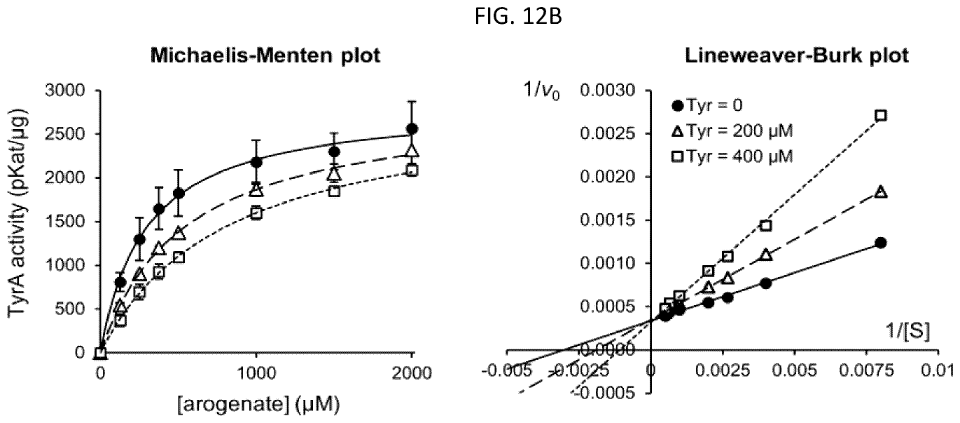

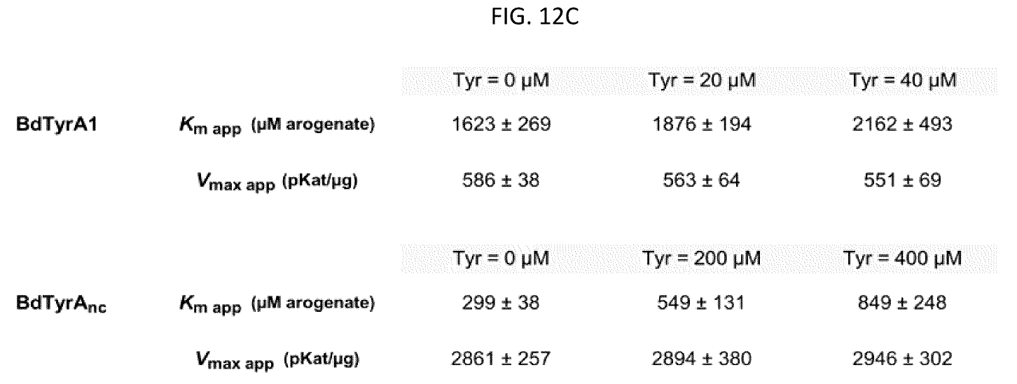

The activity of most plant TyrA enzymes is inhibited competitively at low concentration of tyrosine, generally in the half maximum inhibition (IC₅₀) range of 10 to 50 μM when assayed in vitro¹⁷⁻²⁰. Like other plant TyrA enzymes, BdTyrA1 showed an IC₅₀ for tyrosine at ˜65 μM, and BdTyrA2 had even lower IC₅₀ of ˜20 μM (FIG. 3D, Table 1, FIG. 11). In contrast, BdTyrAnc exhibited a low sensitivity to inhibition by tyrosine, with an estimated IC₅₀ of ˜240 μM. Hence, BdTyrAnc retains >50% of its activity at 200 μM of tyrosine, where BdTyrA1 and BdTyrA2 are fully inactive (FIG. 3D). Despite this marked difference in sensitivity, the inhibition of BdTyrAnc by tyrosine is competitive with arogenate (FIG. 12), as reported for other TyrA enzymes^{19, 20}. Like BdTyrAnc, SbTyrAnc also showed low sensitivity to feedback-inhibition, having a high IC₅₀ for tyrosine of ˜475 μM, whereas SbTyrA1 and SbTyrA2 did not (IC₅₀ at 71 and 44 μM, respectively) (FIG. 3E, Table 1).

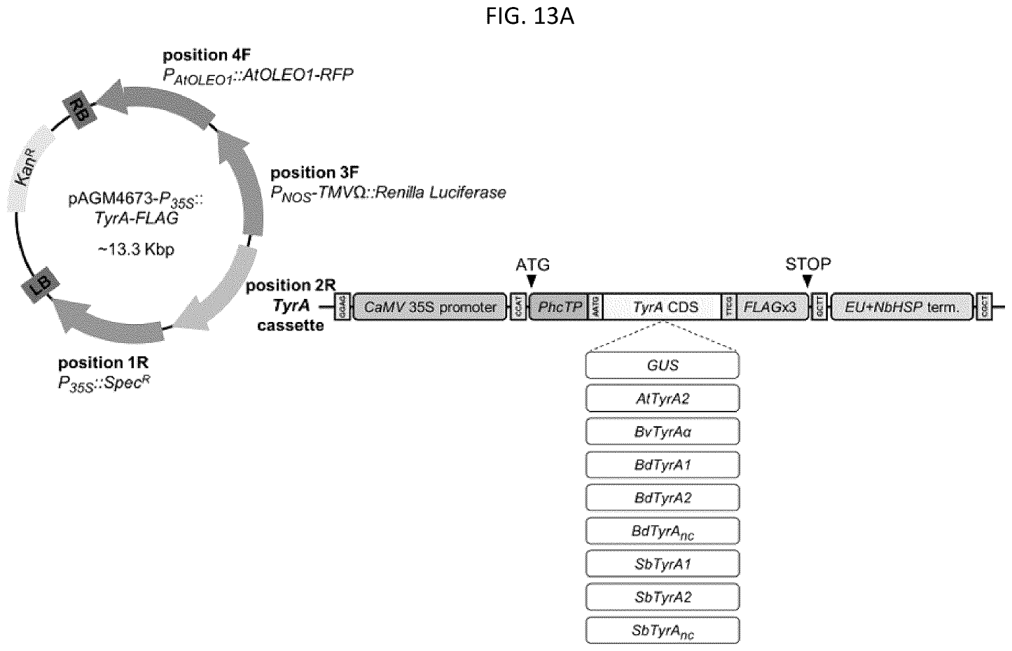

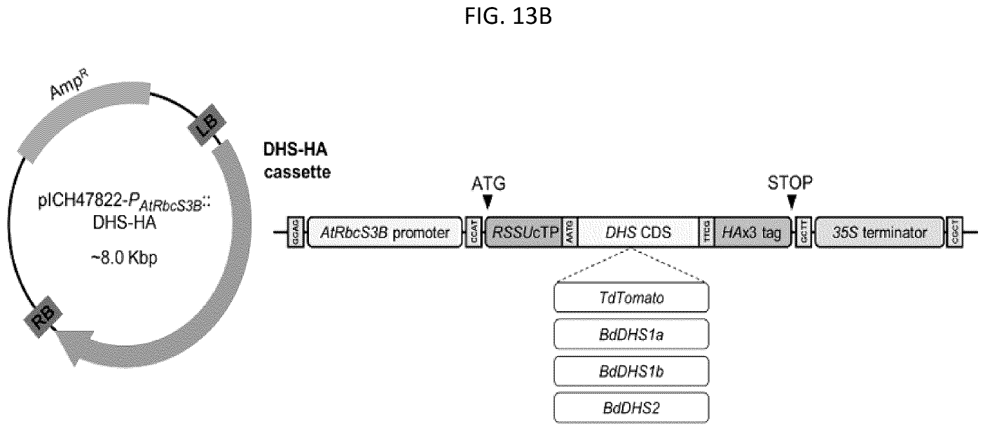

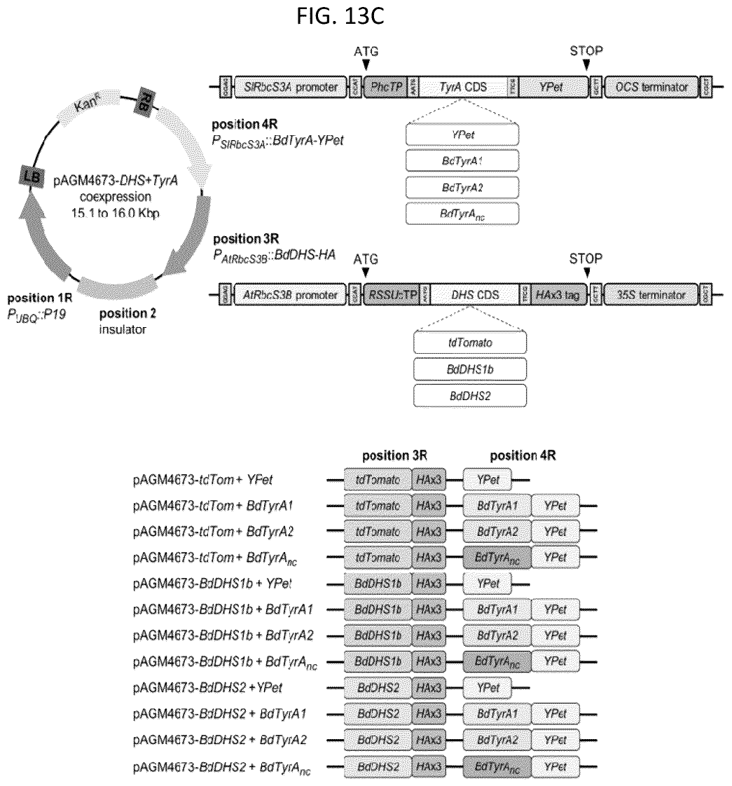

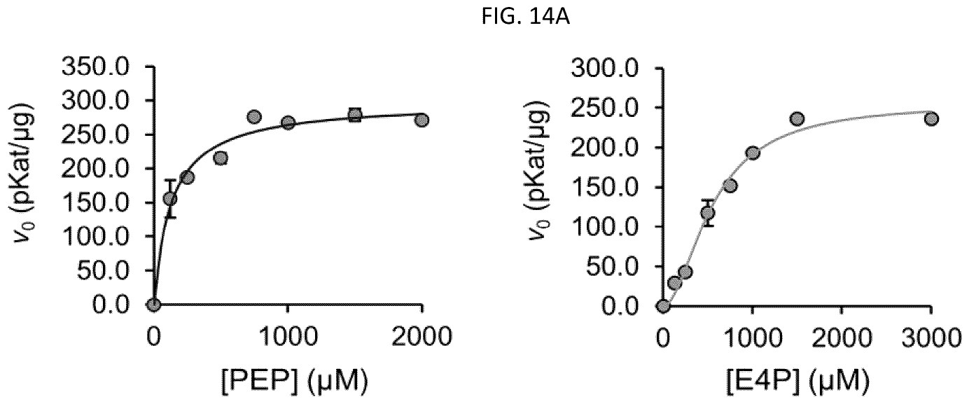

To investigate if the difference in sensitivity to feedback inhibition impacts the activity of the TyrA isoforms in planta, we transiently expressed Brachypodium and Sorghum TyrA genes in Nicotiana benthamiana through Agrobacterium leaf infiltration (FIG. 3F). As controls, β-glucuronidase (GUS), the tyrosine-inhibited AtTyrA2 from Arabidopsis²⁰, and the deregulated BvTyrAα from Beta vulgaris⁴⁵ were also expressed, all under control of the CaMV 35S promoter (P_{CaMV35S}) (FIG. 13). Tyrosine content in the transfected leaves was 2.5 to 3 times higher in BdTyrA1, SbTyrA1, and SbTyrA2 infiltrated leaves compared to the GUS control (FIG. 3F). Similar tyrosine levels were observed in the leaves infiltrated with AtTyrA2. Overexpression of BdTyrA2, which encodes a strongly feedback inhibited enzyme with the lowest IC₅₀ among grass TyrA enzymes (Table 1), did not significantly increase tyrosine levels. In contrast, infiltration with the BdTyrAnc and SbTyrAnc constructs increased tyrosine content by 8 and 14-times relative to the GUS control, respectively, causing an effect similar to that of the deregulated BvTyrAα (FIG. 3F). The in planta accumulation of tyrosine correlated better with the sensitivity of the different TyrA enzymes to feedback inhibition (IC₅₀), rather than the other kinetical parameters (k_{cat}, Kₘ, kcal/Kₘ; Table 1). These results support that, in agreement with their sensitivity to feedback inhibition in vitro, grass TyrAnc, but not TyrA1 or TyrA2, can greatly increase tyrosine production when expressed in planta.

Grasses have a Feedback Insensitive DHS1b Enzyme

Feeding experiments using ¹³CO₂ revealed that, beyond high tyrosine production, grass species also synthesize shikimate and phenylalanine at a higher rate than Arabidopsis (FIG. 1 and FIG. 2). These findings suggest that the regulation of the upstream shikimate pathway may be different in grass species. To test this hypothesis, we characterized the DHS enzymes from Brachypodium and Sorghum, which catalyze a key regulatory step at the entry point of the shikimate pathway (FIG. 1A)^{1, 14, 24}.

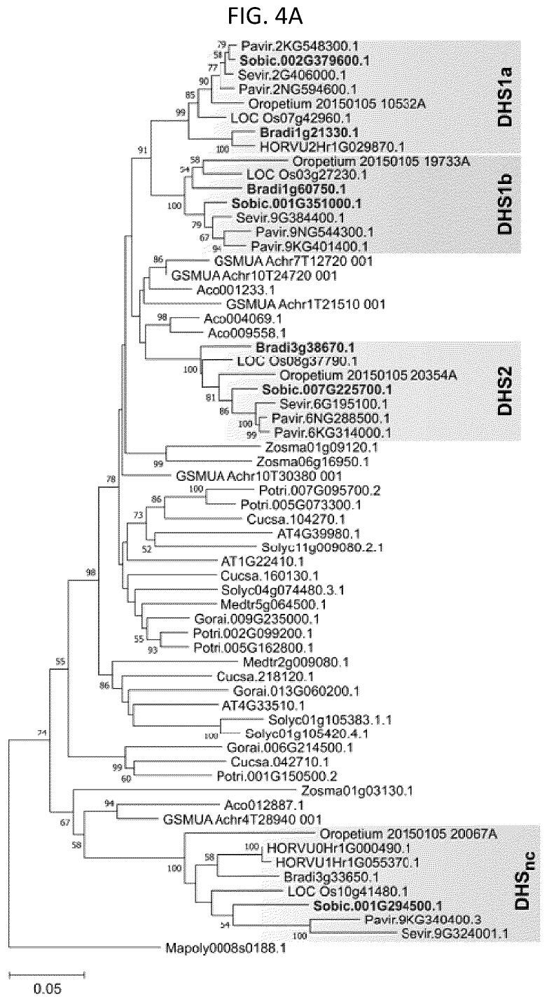

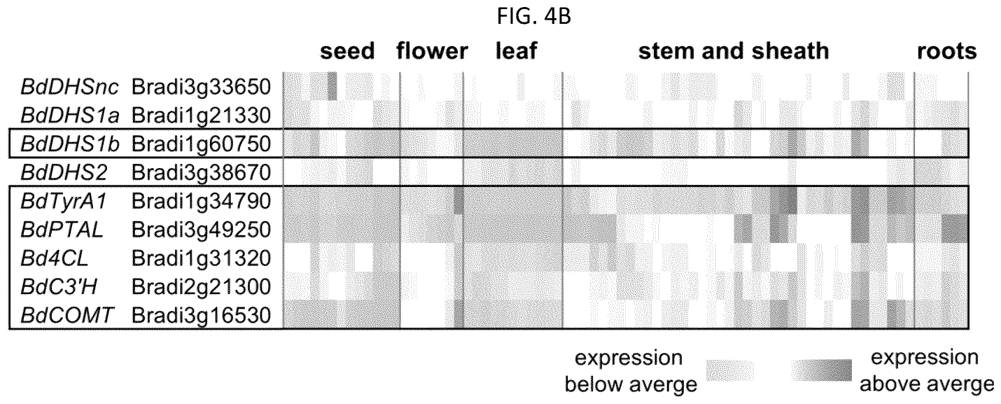

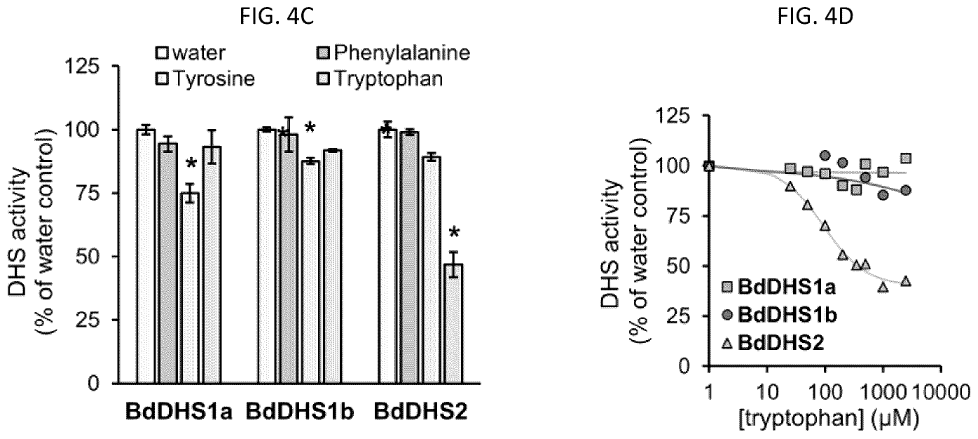

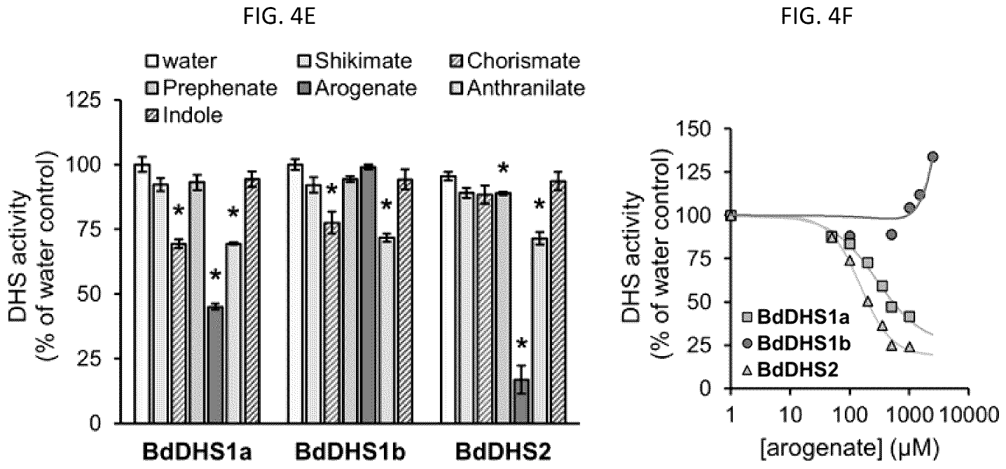

The phylogeny of plant DHS enzymes shows that grasses generally have four DHS isoforms (FIG. 4A), which correspond with the Brachypodium loci Bradi1g21330 (namely BdDHS1a), Bradi1g60750 (BdDHS1b), Bradi3g38670 (BdDHS2), and Bradi3g33650 (BdDHSnc, from non-canonical). Whereas DHS2 and DHSnc are conserved in other monocots, DHS1a and DHS1b (which share ˜90% of protein sequence identity) are likely derived from a gene duplication event within the grass family. The four Brachypodium DHS genes differ in their spatio-temporal expression profile (FIG. 4B). BdDHS2 is dominant in photosynthetic organs, BdDHS1a is expressed across different organs and stages, and BdDHSnc is mostly expressed in seeds. Notably, BdDHS1b expression is induced in the internodes (FIG. 4B), and is co-expressed with BdPTAL, BdTyrA1 and other lignin pathway genes (data not shown)⁴¹.

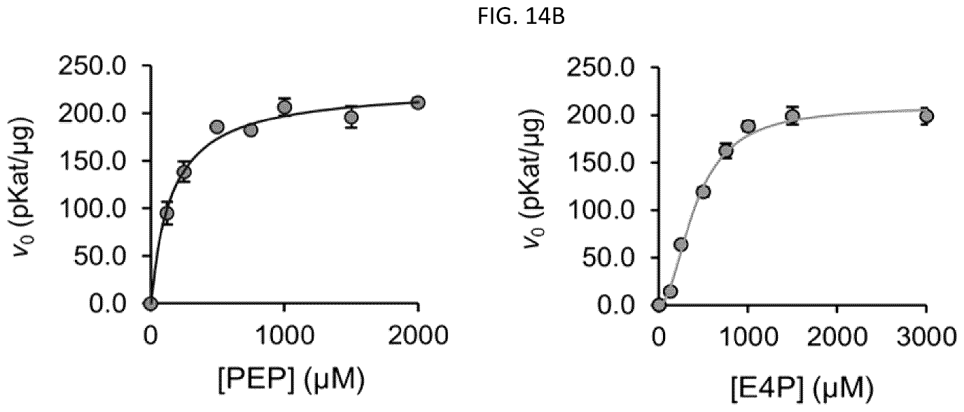

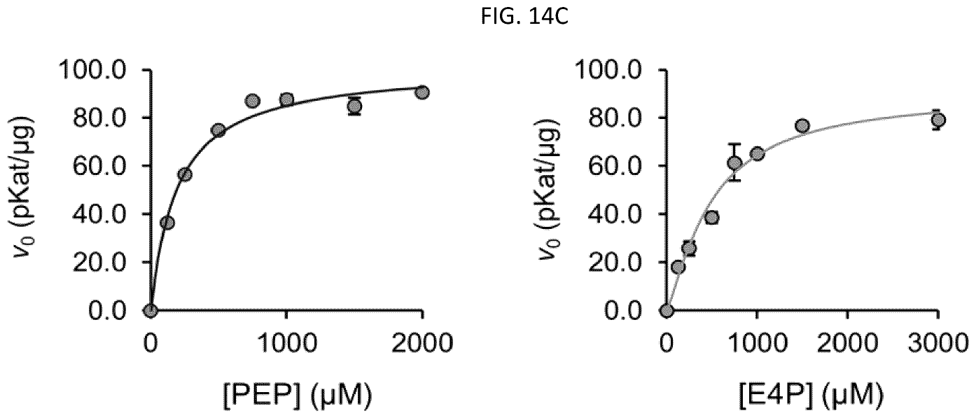

To examine their functional properties, the recombinant DHS enzymes of Brachypodium were produced and characterized in vitro. Though BdDHSnc was also produced, it was not soluble in bacteria and could not be studied. Enzyme assays showed Michaelis-Menten kinetics for phosphoenolpyruvate, with Kₘ values in the range of 135 to 200 μM (Table 2, FIG. 14), but weak to moderate positive cooperativity for erythrose 4-phosphate, with a K_{0.5}—the analogous parameter to Kₘ in cooperative kinetics—in between 400 and 550 μM (Table 2, FIG. 14). k_{cat} values were in the same order of magnitude for the three isoforms (Table 2, FIG. 14). Thus, the three Brachypodium DHS enzymes seem to have similar kinetical parameters.

TABLE 2
Kinetic parameters of DHS enzymes. The data used to determine the kinetic
parameters Kₘ/K_{0.5} and k_{cat} are shown in FIG. 14. k_{cat}/Kₘ was calculated as described
for TyrA enzymes in the legend of Table 1. IC₅₀ for tryptophan and arogenate
were determined based on the original data shown in FIG. 4D and FIG. 4F,
respectively. n.i. = not inhibited. All data are means ± SD of
n = 4-6 derived from at least two independent experiments conducted
on different days using different batches of purified recombinant enzyme.

| | | | | | k_{cat}/Kₘ | k_{cat}/EC₅₀ | | |
|---|---|---|---|---|---|---|---|---|
| | Kₘ | EC₅₀ | H | | (PEP) | (E4P) | IC₅₀ | |
| | PEP | E4P | Coefficient | k_{cat} | (s⁻¹ · | (s⁻¹ · | (μM | (μM |
| | (mM) | (mM) | (only E4P) | (s⁻¹) | mM⁻¹) | mM⁻¹) | Agn) | Trp) |
| BdDHS1a | 0.13 ± | 0.55 ± | 1.8 ± | 17.0 ± | 126.0 ± | 30.7 ± | 285 ± | n.i. |
| | 0.02 | 0.04 | 0.1 | 0.2 | 1.7 | 0.4 | 24 | |
| BdDHS1b | 0.20 ± | 0.40 ± | 2.1 ± | 11.3 ± | 57.2 ± | 28.3 ± | n.i. | n.i. |
| | 0.07 | 0.02 | 0.1 | 0.1 | 0.3 | 0.1 | | |
| BdDHS2 | 0.19 ± | 0.51 ± | 1.5 ± | 5.6 ± | 29.0 ± | 10.8 ± | 91 ± | 120 ± |
| | 0.01 | 0.10 | 0.6 | 0.01 | 0.1 | 0.01 | 11 | 4 |

As recent studies have shown that plant DHS enzymes are feedback-inhibited by multiple effector molecules^{15, 16}, we tested the effect of AAAs and another 14 related metabolites, including various intermediates of the shikimate pathway and the pathways downstream of AAAs, on Brachypodium DHS enzymes. The effect of these compounds was determined at a concentration of 0.5 mM with two alternative methods: real-time spectrophotometric quantification of phosphoenolpyruvate consumption⁴⁹, and final-point quantification of the reaction product, DAHP, by UHPLC-MS.

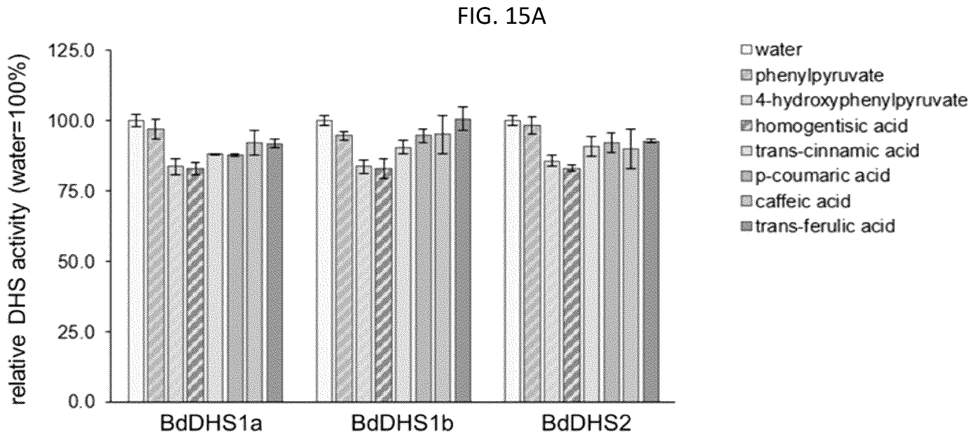

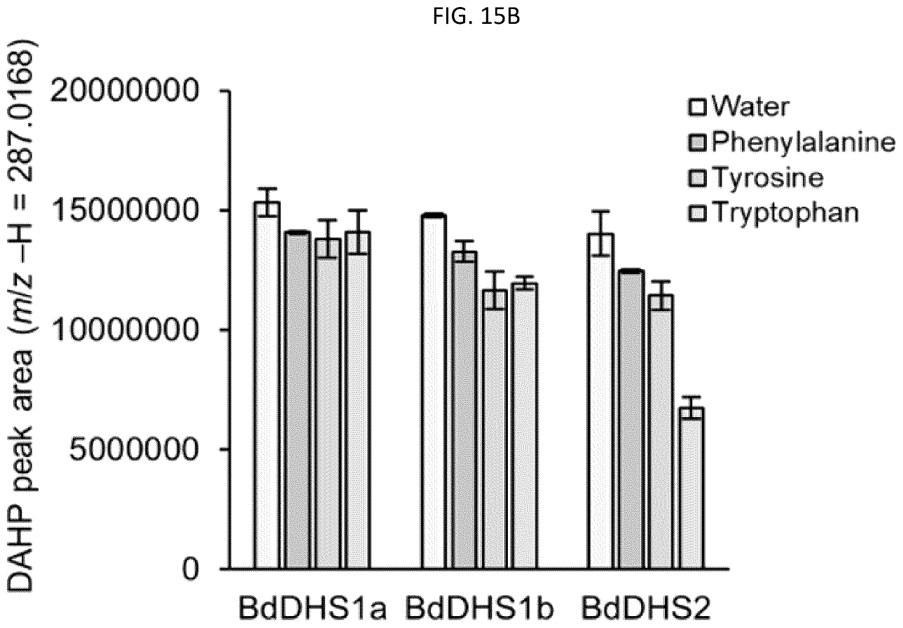

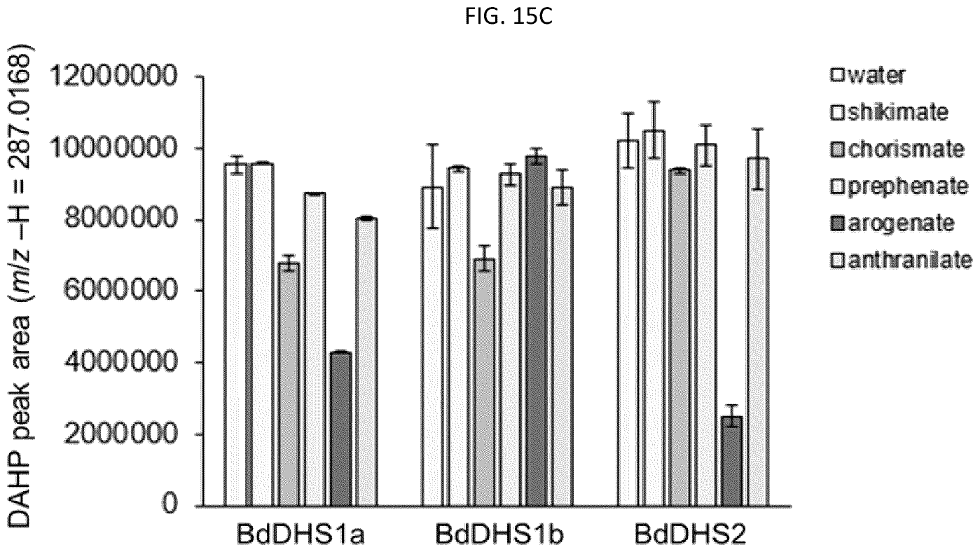

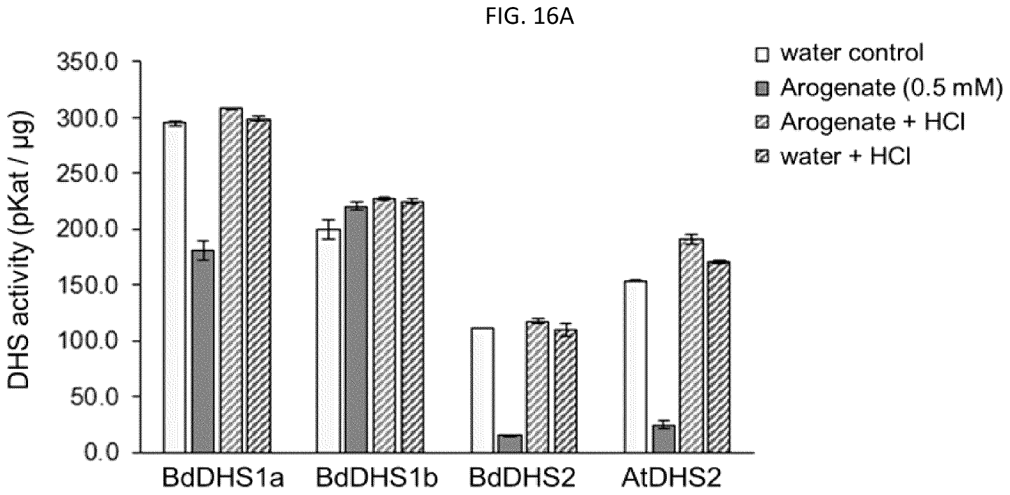

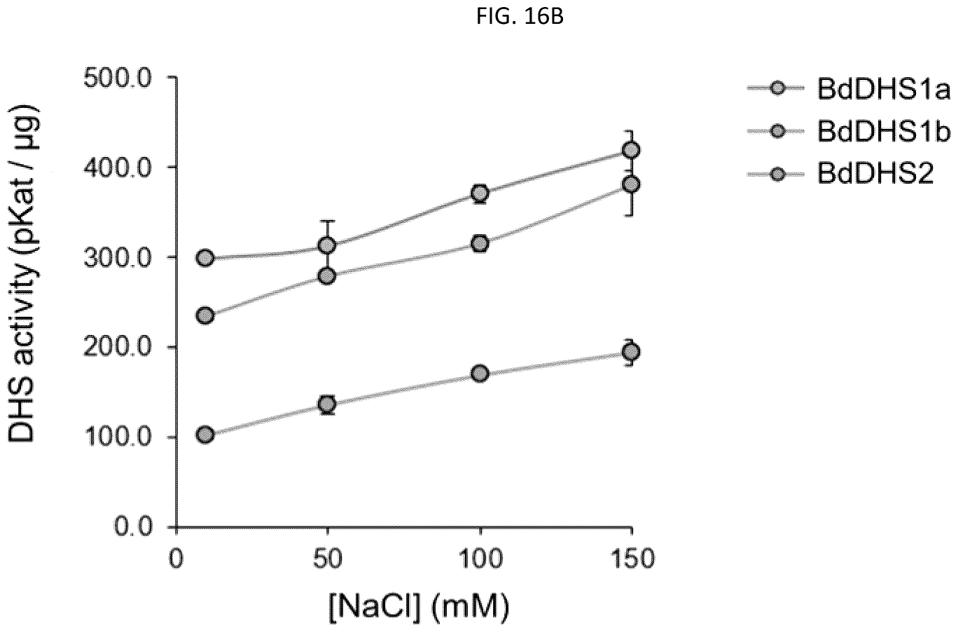

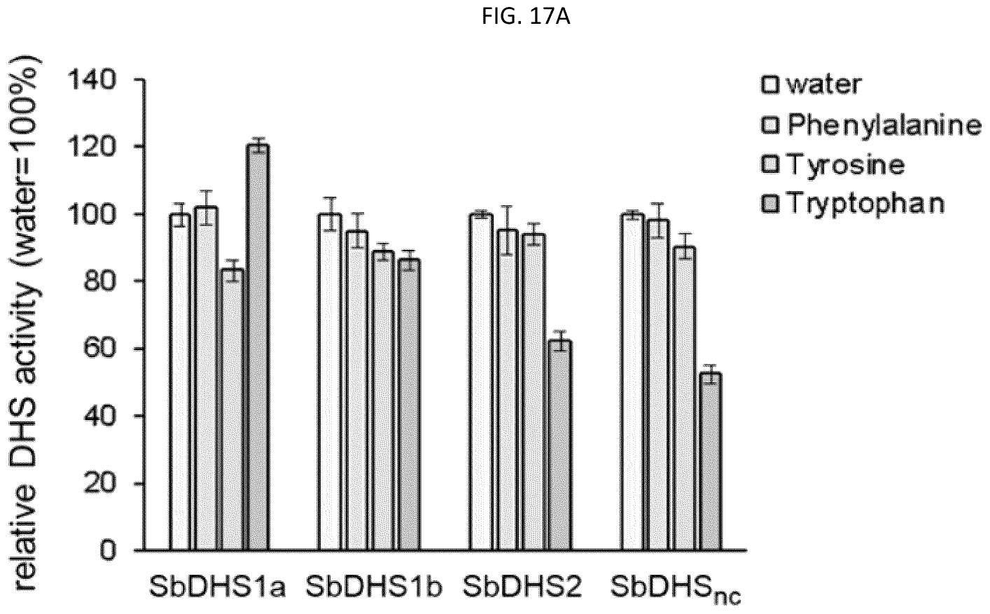

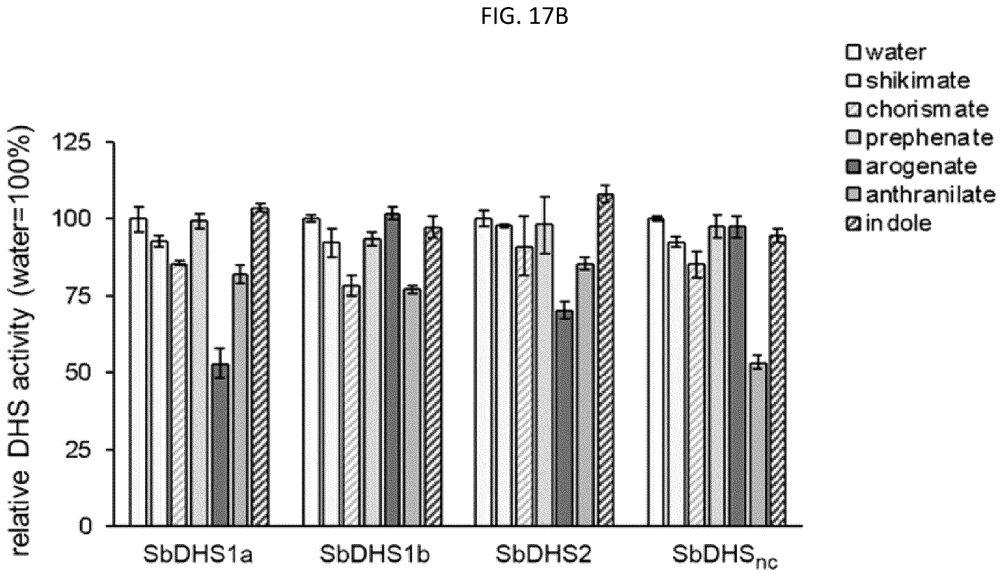

Among the three AAAs, phenylalanine did not cause significant effects on grass DHS activities, which seems to be a common feature in plant DHS enzymes²⁴. Tyrosine, which strongly inhibits Arabidopsis DHS enzymes¹⁵, only caused ˜25% inhibition in BdDHS1a and ˜10% in BdDHS1b and BdDHS2 (FIG. 4C). Conversely, tryptophan strongly inhibited BdDHS2 at an IC₅₀ of ˜120 μM but had no effect on BdDHS1a or BdDHS1b (FIG. 4C, FIG. 4D, Table 2). We did not observe any remarkable inhibitory effect caused by intermediates of AAA catabolism or lignin biosynthesis (FIG. 15). From the different AAA pathway(s) intermediates tested, we observed that arogenate caused the most dramatic effect, causing ˜50% inhibition of BdDHS1a, and >75% in BdDHS2 at 0.5 mM (FIG. 4E, FIG. 15), with a calculated IC₅₀ of ˜285 and ˜91 μM, respectively (FIG. 4F, Table 2). In contrast, BdDHS1b was not inhibited by arogenate even up to 2.5 mM. In fact, DHS1b activity increased in the presence of high concentrations of arogenate (FIG. 4F), likely due to high concentrations of contaminant NaCl present in the arogenate preparation (FIG. 16). Under acidic conditions, arogenate is known to undergo a spontaneous dehydration and decarboxylation into phenylalanine⁵⁰. Arogenate incubated with HCl, which is therefore fully converted into phenylalanine, did not inhibit the DHS enzymes, supporting that the inhibitory compound was arogenate instead of other possible contaminants (FIG. 16). The characterization of the recombinant Sorghum DHS enzymes confirmed that no strong inhibition was caused by tyrosine or phenylalanine, whereas 0.5 mM of tryptophan caused 40 to 50% inhibition of SbDHS2 and SbDHSnc (FIG. 17). Like in Brachypodium DHS enzymes, 0.5 mM arogenate inhibited SbDHS1a and SbDHS2 at ˜50% and ˜40%, respectively, but had no effect on SbDHS1b.

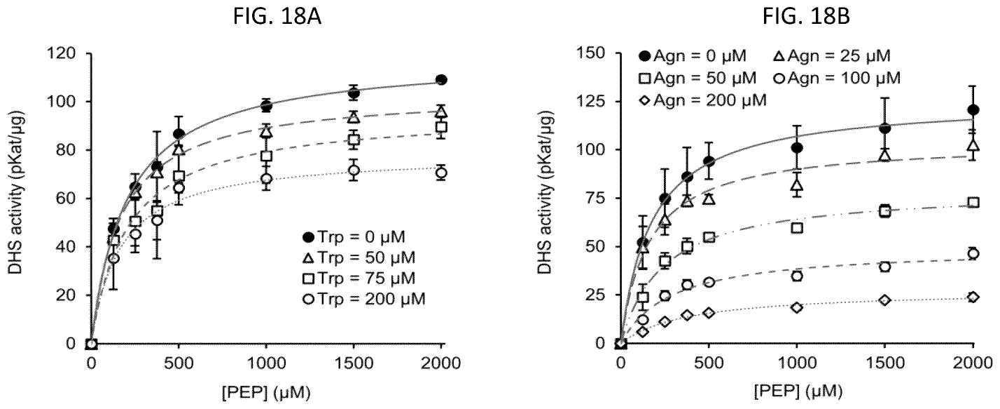

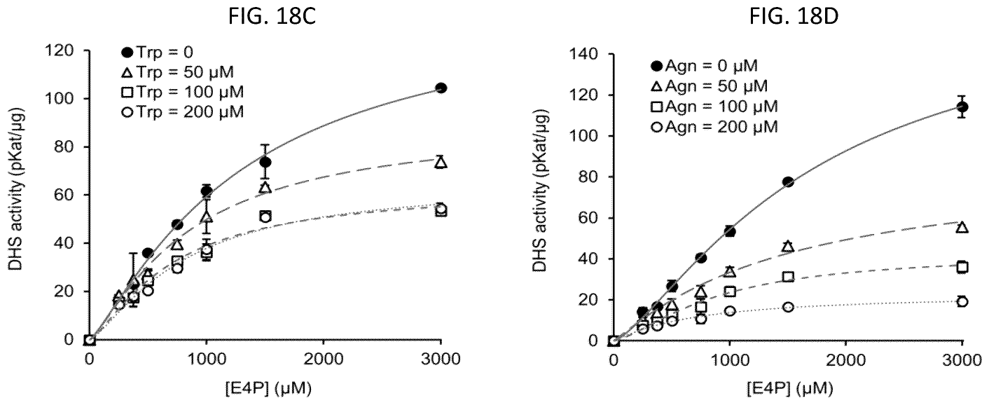

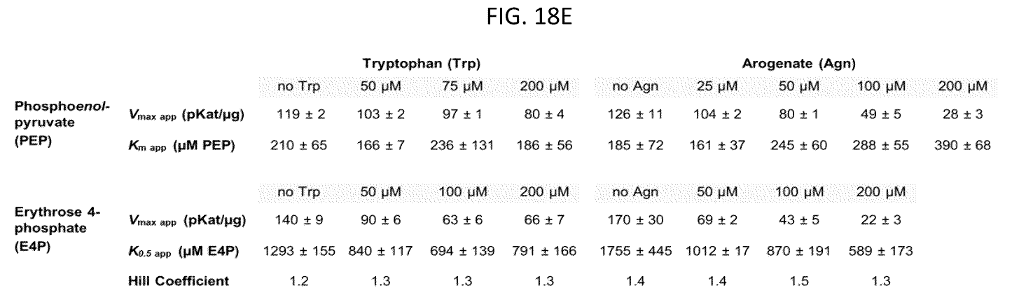

Determination of the kinetic parameters of BdDHS2 at different concentrations of tryptophan and arogenate showed that both effectors decrease Vₘₐₓ but had distinct impacts on Kₘ or EC₅₀. For PEP, tryptophan did not cause a significant change in the Kₘ, which is indicative of non-competitive inhibition, but arogenate caused the Kₘ to increase, indicating a mixed inhibition mechanism (FIG. 18). In respect to E4P, both tryptophan and arogenate decreased K_{0.5}, suggesting uncompetitive inhibition kinetics (FIG. 18). These findings resemble previous studies from bacterial type-II DHS enzymes⁵¹ and support that type-II DHS enzymes, which include plant DHS enzymes, are allosteric enzymes with a complex response to the binding of their substrates and effectors.

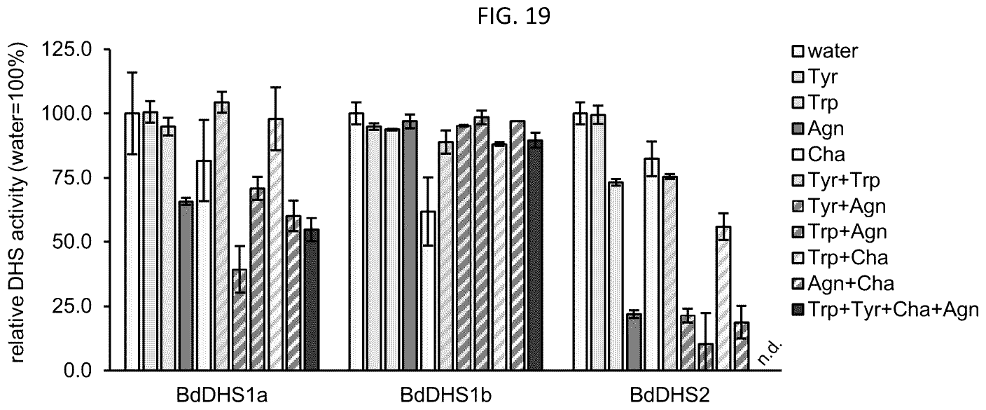

DHS effector molecules can have synergistic effects when combined in vitro⁵¹. To explore this possibility, we tested the impact of different combinations of tryptophan, tyrosine, arogenate and chorismate, at 0.15 mM each, on the activity of Brachypodium DHS enzymes. Although most combinations did not exhibit strong additive effects, some of the combinations, such as tryptophan plus arogenate for BdDHS2 and tyrosine plus arogenate for BdDHS1a, caused additional inhibition (FIG. 19). None of the combinations tested had a significant impact on BdDHS1b (FIG. 19). Hence, although grass DHS1a is inhibited by arogenate and DHS2 by both arogenate and tryptophan, DHS1b seems largely insensitive to feedback inhibition in vitro.

Co-Expression of BdDHS1b and BdTyrA1 Synergistically Enhances Tyrosine Production while Maintaining High Phenylalanine Production

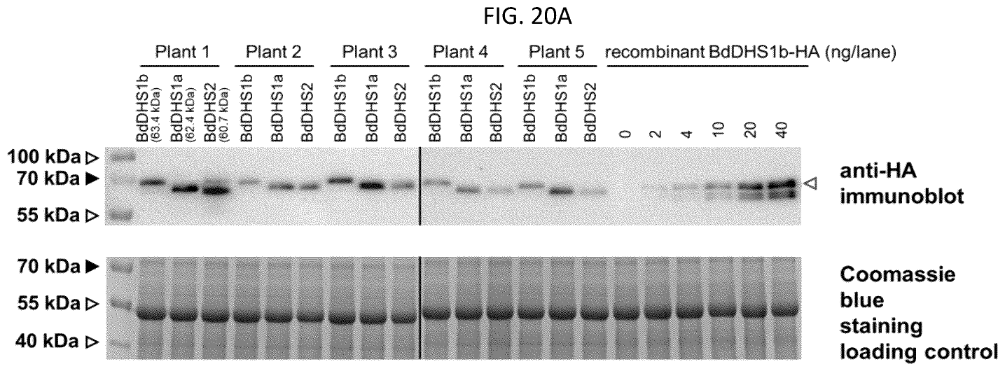

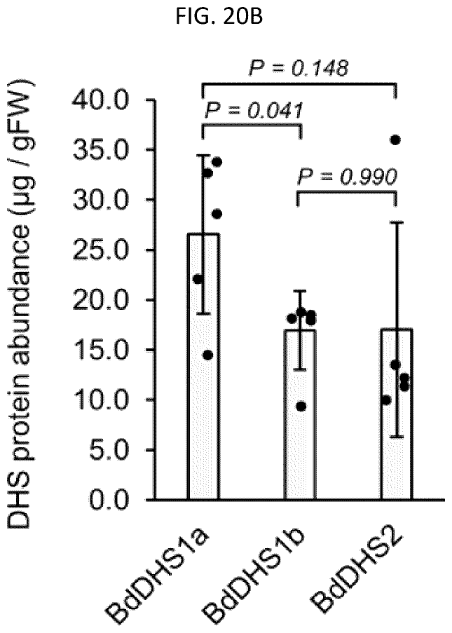

To evaluate in planta how DHS biochemical regulation may impact the production of AAAs, we expressed BdDHS1a, BdDHS1b, and BdDHS2 in Nicotiana benthamiana leaves under control of the Arabidopsis RuBisCO S3B promoter (P_{AtRbcS3B}), which provides 15-20% of the expression level of CaMV 35S promoter⁵² (FIG. 5A). Transient expression of BdDHS1a and BdDHS2, both sensitive to feedback inhibition in vitro, did not significantly alter the content of phenylalanine, tyrosine, tryptophan, nor their common precursor shikimate, compared to the control expressing tdTomato (tdTom) (FIG. 5A). To the contrary, the expression of BdDHS1b triggered the accumulation of 10-times more tyrosine, 3-times more shikimate and tryptophan, and 18-times more phenylalanine, which was the most abundant AAA (˜2000 nmol/gFW; FIG. 5A). Quantification of the BdDHS-HA tagged proteins by immunoblotting showed that, despite the marked differences in metabolite levels, the protein levels of these three DHS isoforms were comparable (FIG. 20). Taken together, these results support in vitro results showing that grasses possess a naturally deregulated DHS1b that can boost AAA production, mostly phenylalanine, when expressed heterologously in planta.

Gene expression data in Brachypodium (FIG. 4B) indicate that BdTyrA1 is co-expressed with BdDHS1b and BdPTAL in the internodes, where we detected a high rate of tyrosine production (FIG. 2). Nevertheless, BdTyrA1 is strongly inhibited by tyrosine in vitro (FIG. 3D) and its expression alone in Nicotiana leaves had little impact on tyrosine levels (FIG. 3F). We therefore hypothesized that BdTyrA1 and BdDHS1b may co-operate in the high production of tyrosine and phenylalanine observed in grass tissues. To test this possibility, we co-expressed in planta different combinations of BdTyrA enzymes and BdDHS enzymes and measured the impact on phenylalanine and tyrosine levels. To this end, we took advantage of the Golden Gate modular cloning system⁵² and assembled the BdTyrA and BdDHS expression cassettes into the same vector backbone (FIG. 13). To avoid causing a strong over-expression, BdTyrA and BdDHS were driven by RuBisCO small subunit promoters from Arabidopsis (P_{AtRbcS3B}) and tomato (P_{SIRbcS3A}), respectively (FIG. 13)⁵¹. Consistent with the results shown in FIG. 3F, the expression of BdTyrAnc, but not BdTyrA1 or BdTyrA2, together with the tdTom control, led to a strong increase in tyrosine levels (˜100-times) (FIG. 5B). Notably, BdTyrAnc expression decreased phenylalanine levels by 4-times compared to the negative control co-expressing YPet with tdTom (FIG. 5B). Co-expression of BdTyrAnc with BdDHS1b showed additive effects and further increased tyrosine to a dramatic ˜400-times the tdTom+YPet control, while still negatively impacting phenylalanine level (FIG. 5B). Interestingly, co-expression of feedback-regulated BdTyrA1 together with BdDHS1b boosted tyrosine content to a level similar to BdTyrAnc, while still maintaining a high production of phenylalanine (FIG. 5B). This synergistic effect was not observed upon co-expression of BdDHS1b with BdTyrA2, possibly due to the tight feedback regulation of BdTyrA2 (Table 1). Similarly, no significant additive effects were observed upon co-expression of the BdTyrA enzymes with feedback-regulated BdDHS2 (FIG. 5B). These results show that simultaneous expression of deregulated BdDHS1b with the feedback-regulated enzyme BdTyrA1 can render high levels of both tyrosine and phenylalanine in planta.

### DISCUSSION

The emergence of phenylpropanoid metabolism is a key adaptation during the transition of plants from water to land, conferring plants with enhanced mechanical strength, and protection against UV radiation and desiccation. Phenylpropanoids are remarkably diverse across the plant phylogeny and are synthesized exclusively from phenylalanine and by the PAL enzyme in almost all plant groups^{5, 53, 54}. Grasses are an exception, as they use the PTAL reaction to synthesize phenylpropanoids from tyrosine, which constitutes a shortcut in the “canonical” phenylpropanoid pathway (FIG. 1)^{5, 55}. In this study, we used this unique grass feature to investigate how enzyme evolution re-shapes metabolic flows and regulation at the interface of primary metabolism and the downstream natural product pathways.

Labeling experiments using ¹³CO₂ provided well-grounded evidence that supports high tyrosine production in grasses, especially in the internodes (FIG. 1 and FIG. 2). Consistently, the expression of TyrA genes was induced in lignifying tissues of grasses (FIG. 3), unlike in dicots where ADT but not TyrA genes are co-expressed with lignin pathway genes^{29, 56, 57}. One of the grass TyrA enzymes, TyrAnc, also showed low sensitivity to inhibition by tyrosine in vitro, and its expression in Nicotiana benthamiana boosted tyrosine levels (FIG. 3F and FIG. 5B), though at the expense of phenylalanine production (FIG. 5B). These findings clearly support that, unlike most plant TyrA enzymes¹⁸⁻²⁰, grass TyrAnc has low sensitivity to feedback inhibition. Partially feedback insensitive TyrA enzymes have been previously described in Caryophyllales, with IC₅₀ values between ˜400 and 700 μM of tyrosine^{45, 58}, and in legumes, where the prephenate dehydrogenase TyrAp is completely feedback insensitive^{40, 46}. However, phylogenetic evidence shows that grass TyrAnc enzymes are not related to the Caryophyllales TyrAα enzyme and legume TyrAp enzyme (FIG. 3A), indicating that feedback deregulated TyrA enzymes likely evolved independently in these three plant groups.

Despite their highly active tyrosine biosynthesis, grasses still maintain a high rate of phenylalanine production, particularly in stems (FIG. 1 and FIG. 2). Detailed characterization of grass DHS isoforms further revealed the presence of DHS1b which, unlike DHS1a and DHS2, was insensitive to feedback inhibition (FIG. 4). The expression of DHS1b, but not DHS1a or DHS2, in Nicotiana leaves also boosted the level of shikimate, tryptophan, tyrosine and, especially phenylalanine (FIG. 5A). Although feedback-insensitive bacterial DHS enzymes have been introduced in plants to increase the production of AAAs and/or phenylpropanoids^{21, 32, 59-61} DHS1b constitutes, to our knowledge, the first report of a naturally occurring deregulated plant DHS.

Previous studies on biochemical characterization of DHS activity from plant extracts-most of them dicots-reported varying observations about the sensitivity of DHS to feedback regulation²⁴, likely due to the presence of multiple DHS isoforms in plants^{24, 62}. In monocots, a single study found that DHS activity in crude extracts from 9-day-old maize plantlets is inhibited by tryptophan, but not by phenylalanine or tyrosine⁶³, consistent with a predominant role of the tryptophan-inhibited DHS2 in green tissues (FIG. 4). Based on the differences in feedback regulation and expression patterns of individual grass DHS isoforms (FIG. 4), DHS1a and DHS2 may display more general or housekeeping roles, whereas DHS1b may have organ-specific (e.g., internode) functions that demand high AAA production. Recent studies in Arabidopsis also showed a complex isoform-dependent feedback regulation where different DHS isoforms are expressed and regulated differently^{15, 16}. Moreover, Arabidopsis DHS enzymes were inhibited by chorismate, caffeic acid, tyrosine, and its metabolites 4-hydroxyphenylpyruvate and homogentisic acid³⁹, which all had little effect on grass DHS enzymes (FIG. 4, FIG. 15, and FIG. 17). In the case of tyrosine and its derivatives 4-hydroxyphenylpyruvate and homogentisic acid, the insensitivity of grass DHS enzymes might be linked to the high tyrosine levels present in grass tissues (FIG. 1 and FIG. 2). Therefore, it seems that the feedback regulation of plant DHS enzymes is not only isoform-dependent, but also species-dependent. These findings indicate that both biochemical and transcriptional regulatory mechanisms targeting DHS enzymes give plant species a precise yet adaptable tool to modulate AAA production.

Although the feedback insensitive TyrAnc is likely contributing to the high rate of tyrosine production in grass internodes (FIG. 2), it is somewhat incongruent that TyrA1, expression of which is strongly induced in the internodes (FIG. 3C), encodes a tyrosine-inhibited enzyme (Table 1). Interestingly, the combinatorial expression of the feedback inhibited TyrA1 with the deregulated DHS1b led to high production of both tyrosine and phenylalanine in planta (FIG. 5). This synergistic effect might be a consequence of “pushing” the carbon flow into the shikimate pathway, causing an accumulation of arogenate that would alleviate the competitive feedback inhibition of TyrA1 by tyrosine (FIG. 12)^{19, 20} and support high rates of both tyrosine and phenylalanine production. The relatively high Kₘ values of grass TyrA1 enzymes for arogenate (Table 1) may reflect their specialization for working at the high substrate levels provided by DHS1b, while TyrAnc is possibly better suited for producing tyrosine at low arogenate concentrations thanks to its lower Kₘ value (Table 1).

Overall, the current findings highlight that the interplay between feedback-regulated (TyrA1) and deregulated (DHS1b, TyrAnc) enzymes at the entry and exit steps of AAA biosynthesis can maintain the high production of both tyrosine and phenylalanine. This fine-tuning of the upstream AAA pathway likely supports the unique dual lignin pathway found in grasses. Future studies of these key enzymes from different monocot species will address the evolutionary history of the coordinated regulation of the grass AAA and lignin pathways. This fundamental knowledge also provides useful genetic tools for the rationale engineering of plant primary metabolism to support the production of aromatic products.

### Materials and Methods

### Plant Materials and Growth Conditions

The following grass cultivars were used in this study: Brachypodium distachyon 21-3, Sorghum bicolor RTx430, and Setaria viridis A10.1.

Arabidopsis, Brachypodium, and Setaria plants used for ¹³CO₂ feeding and RT-qPCR analysis were kept in a growth chamber at 22° C., 12h-photoperiod under ˜100 μE of light intensity, 60% humidity, and watered with a 1:10 dilution of Hoagland's solution.

Nicotiana benthamiana plants used for transient expression experiments were grown at 22° C. in a 12h-photoperiod under ˜200 μE of light intensity, 60% humidity, and watered with a 12:4:8 (N:P:K) plant nutritive solution (Miracle-Gro) at a 1:1000 dilution.

### Gene Expression Analysis and RT-qPCR

Spatiotemporal gene expression data from Brachypodium distachyon and Sorghum bicolor were retrieved from the databases PlaNet⁴¹ and MOROKOSHI⁴², respectively.

Total RNA was isolated from young leaves and developing internodes of 1.5-month-old Brachypodium and Setaria plants using RNeasy Plant Mini Kit (Qiagen), following the manufacturer's instructions. RNA was treated with RQ1 RNase-free DNase (Promega) and was reverse transcribed with M-MLV Reverse Transcriptase (Promega) using random hexamer primers. Quantitative PCR analysis was carried out in a Stratagene Mx3000P (Agilent Technologies) thermocycler using GoTaq qPCR Master Mix (Promega). Ct values were determined using LinRegPCR⁶⁴ version 2018.0. Primers used are listed in Table 3. Ct values were converted into mass of template by using a calibration curve made of the corresponding RT-qPCR amplicon, cloned into the EcoRV site of pML94 vector using conventional blunt-end ligation protocols. Ubiquitin ligase genes of Brachypodium and Setaria were chosen as reference genes based on previous publications⁶⁵.

TABLE 3
Primers

| Primer | | | |
|---|---|---|---|
| Name | Sequence (from 5′-) | Target gene | Purpose |
| pHM532Bd3 | GATCTAGACTCGAGGGT | BdTyrA1 | In-Fusion cloning into |
| 47pML94F | ACCATGCTGTCGTCTTCC | | pML94 for transient |
| | (SEQ ID NO: 45) | | expression in Arabidopsis |
| | | | protoplasts |
| pHM533Bd3 | CTAGTGCATGCGGCCGC | BdTyrA1 | In-Fusion cloning into |
| 47pML94R | ATTCCGGACGGTGG | | pML94 for transient |
| | (SEQ ID NO: 46) | | expression in Arabidopsis |
| | | | protoplasts |
| pHM534Bd3 | CGCGCGGCAGCCATATG | BdTyrA1 | In-Fusion cloning into |
| 47pET28aF | CGCGCCATCGACGC | | NdeI/BamHI sites of |
| | (SEQ ID NO: 47) | | pET28a |
| pHM535Bd3 | GCTCGAATTCGGATCCC | BdTyrA1 | In-Fusion cloning into |
| 47pET28aR | TAATTCCGGACGGTGG | | NdeI/BamHI sites of |
| | (SEQ ID NO: 48) | | pET28a |
| pHM536Bd3 | GATCTAGACTCGAGGGT | BdTyrA2 | In-Fusion cloning into |
| 48pML94F | ACCATGTCTTCCTCCGGT | | pML94 for transient |
| | C (SEQ ID NO: 49) | | expression in Arabidopsis |
| | | | protoplasts |
| pHM537Bd3 | CTAGTGCATGCGGCCGC | BdTyrA2 | In-Fusion cloning into |
| 48pML94R | CCGTCCGTCATCCAA | | pML94 for transient |
| | (SEQ ID NO: 50) | | expression in Arabidopsis |
| | | | protoplasts |
| pHM538Bd3 | CGCGCGGCAGCCATATG | BdTyrA2 | In-Fusion cloning into |
| 48pET28aF | CGTGCCACGGACGC | | NdeI/BamHI sites of |
| | (SEQ ID NO: 51) | | pET28a |
| pHM539Bd3 | GCTCGAATTCGGATCCT | BdTyrA2 | In-Fusion cloning into |
| 48pET28aR | CACCGTCCGTCATCC | | NdeI/BamHI sites of |
| | (SEQ ID NO: 52) | | pET28a |
| pHM540Bd3 | GATCTAGACTCGAGGGT | BdTyrAnc | In-Fusion cloning into |
| 9pML94F | ACCATGGCTTCCTCCCTT | | pML94 for transient |
| | G (SEQ ID NO: 53) | | expression in Arabidopsis |
| | | | protoplasts |
| pHM541Bd3 | CTAGTGCATGCGGCCGC | BdTyrAnc | In-Fusion cloning into |
| 9pML94R | AATGGGGACCCTCTCT | | pML94 for transient |
| | (SEQ ID NO: 54) | | expression in Arabidopsis |
| | | | protoplasts |
| pHM542Bd3 | CGCGCGGCAGCCATATG | BdTyrAnc | In-Fusion cloning into |
| 9pET28aF | GCCGAGCAGGAGCAA | | NdeI/BamHI sites of |
| | (SEQ ID NO: 55) | | pET28a |
| pHM543Bd3 | GCTCGAATTCGGATCCTT | BdTyrAnc | In-Fusion cloning into |
| 9pET28aR | AAATGGGGACCCTCTC | | NdeI/BamHI sites of |
| | (SEQ ID NO: 56) | | pET28a |
| pHM1751- | TCACTCTGTGGTCTCAAA | SbTyrA1 | In-Fusion cloning into |
| SbTyrA1GG | TGCGCGCGCTGGACGCC | | pAGM1287 BsaI sites as |
| F | GCCC (SEQ ID NO: 57) | | Golden Gate level 0 part |
| pHM1752- | CCACTTCGTGGTCTCACG | SbTyrA1 | In-Fusion cloning into |
| SbTyrA1GG | AACTCTTGCGGACGTTG | | pAGM1287 BsaI sites as |
| R | GAGGAG (SEQ ID NO: 58) | | Golden Gate level 0 part |
| pHM1753- | TCACTCTGTGGTCTCAAA | SbTyrA2 | In-Fusion cloning into |
| SbTyrA2GG | TGCGCGCCACGGGTGCC | | pAGM1287 BsaI sites as |
| F | TCGC (SEQ ID NO: 59) | | Golden Gate level 0 part |
| pHM1754- | CCACTTCGTGGTCTCACG | SbTyrA2 | In-Fusion cloning into |
| SbTyrA2GG | AACTATTATTCTCCTCTC | | pAGM1287 BsaI sites as |
| R | CCGACTTG (SEQ ID NO: | | Golden Gate level 0 part |
| | 60) | | |
| pHM1755- | TCACTCTGTGGTCTCAAA | SbTyrAnc | In-Fusion cloning into |
| SbTyrA3GG | TGAGCCCGCCCGCCGCC | | pAGM1287 BsaI sites as |
| F | ACCGC (SEQ ID NO: 61) | | Golden Gate level 0 part |
| pHM1756- | CCACTTCGTGGTCTCACG | SbTyrAnc | In-Fusion cloning into |
| SbTyrA3GG | AACTTAAGAGGCGAGCT | | pAGM1287 BsaI sites as |
| R | GCAGGAG (SEQ ID NO: | | Golden Gate level 0 part |
| | 62) | | |
| pHM1757- | ATGGCCTCCTCGCTCCGC | SbTyrAnc | Nested PCR for Sorghum |
| SbTyrA3nest | C (SEQ ID NO: 63) | | TyrAnc |
| F | | | |
| pHM1758- | GTATCCGGTTGAAGTGT | SbTyrAnc | Nested PCR for Sorghum |
| SbTyrA3nest | AGG (SEQ ID NO: 64) | | TyrAnc |
| R | | | |
| pHM2274_ | CACCACCGTCCGGAATT | BdTyrA1 | RT-qPCR |
| BdTyrA1_qF | AGC (SEQ ID NO: 65) | | |
| pHM2275_ | GCACCAGTTTCTCCCCA | BdTyrA1 | RT-qPCR |
| BdTyrA1_qR | AAG (SEQ ID NO: 66) | | |
| pHM2316 | GATGACGGACGGTGATC | BdTyrA2 | RT-qPCR |
| BdTyrA2 | TCG (SEQ ID NO: 67) | | |
| qF2 | | | |
| pHM2317 | TTCGTACCGCTTGTTGGT | BdTyrA2 | RT-qPCR |
| BdTyrA2 | CG (SEQ ID NO: 68) | | |
| qR2 | | | |
| pHM2278_ | TGCTGTGTCCCCTCTCCT | BdTyrAnc | RT-qPCR |
| BdTyrA3_qF | C (SEQ ID NO: 69) | | |
| pHM2279_ | AGGGCTGAAAGACACTG | BdTyrAnc | RT-qPCR |
| BdTyrA3_qR | GGC (SEQ ID NO: 70) | | |
| pHM2318 | CAGACAATGCGGAGATG | SvTyrA2 | RT-qPCR |
| SvTyrA2 qF2 | ATCG (SEQ ID NO: 71) | (Sevir.4G287000.1) | |
| pHM2319 | TTTGCTTCAGAAACCAT | SvTyrA2 | RT-qPCR |
| SvTyrA2 | GTCAC (SEQ ID NO: 72) | (Sevir.4G287000.1) | |
| qR2 | | | |
| pHM2282_ | GGTAGTAATTCAGTGCG | SvTyrA1 | RT-qPCR |
| SvTyrA1_qF | TCGG (SEQ ID NO: 73) | (Sevir.4G286800.1) | |
| pHM2283_ | GGTGTTCTTCTTCCAGAG | SvTyrA1 | RT-qPCR |
| SvTyrA1_qR | AGG (SEQ ID NO: 74) | (Sevir.4G286800.1) | |
| pHM2284_ | GATCGCTTCCATCCCAA | SvTyrAnc | RT-qPCR |
| SvTyrA3_qF | GGC (SEQ ID NO: 75) | (Sevir.4G143200.1) | |
| pHM2285_ | CAGGCGGTCTGAAAGGA | SvTyrAnc | RT-qPCR |
| SvTyrA3_qR | AGG (SEQ ID NO: 76) | (Sevir.4G143200.1) | |
| pHM2290_ | AGTTGTCGCGTGTCTGA | Bradilg32860.3 | RT-qPCR, reference gene |
| BdUBI10_qF | GTC (SEQ ID NO: 77) | | Polyubiquitin 10 |
| pHM2291_ | ACACGGGCTCACTTATT | Bradilg32860.3 | RT-qPCR, reference gene |
| BdUBI10_qR | CATC (SEQ ID NO: 78) | | Polyubiquitin 10 |
| pHM2294_ | GGGCTCATTGTGCTGCT | Sevir.5G079801.1 | RT-qPCR, reference gene |
| SvUBI4_qF | GTC (SEQ ID NO: 79) | | Polyubiquitin 4 |
| pHM2295_ | CCGGAGGACATAGGACT | Sevir.5G079801.1 | RT-qPCR, reference gene |
| SvUBI4_qR | TGC (SEQ ID NO: 80) | | Polyubiquitin 4 |
| pHM2178 | GGTGCCGCGCGGCAGCC | BdDHS1b | In-Fusion cloning into |
| BdDHS1b | ATATGGCCGTCCACGCC | | NdeI/BamHI sites of |
| Fwd | GCGGAGCC (SEQ ID NO: | | pET28a |
| | 81) | | |
| pHM2179 | CGGAGCTCGAATTCGGA | BdDHS1b | In-Fusion cloning into |
| BdDHS1b | TCCTCAGAAACCATAGG | | NdeI/BamHI sites of |
| Rvs | TTGGCAATG (SEQ ID | | pET28a |
| | NO: 82) | | |
| pHM2180 | GGTGCCGCGCGGCAGCC | BdDHS1a | In-Fusion cloning into |
| BdDHS1a | ATATGGCCGTGCACGCC | | NdeI/BamHI sites of |
| Fwd | GCCGACCC (SEQ ID NO: | | pET28a |
| | 83) | | |
| pHM2181 | CGGAGCTCGAATTCGGA | BdDHS1a | In-Fusion cloning into |
| BdDHS1a | TCCTTAGAAGGCCAATG | | NdeI/BamHI sites of |
| Rvs | GCGGCAGTG (SEQ ID | | pET28a |
| | NO: 84) | | |
| pHM2184 | GGTGCCGCGCGGCAGCC | BdDHS2 | In-Fusion cloning into |
| BdDHS2 | ATATGATCCGCGCGCAC | | NdeI/BamHI sites of |
| Fwd | GCGGTGCG (SEQ ID NO: | | pET28a |
| | 85) | | |
| pHM2185 | CGGAGCTCGAATTCGGA | BdDHS2 | In-Fusion cloning into |
| BdDHS2 | TCCTCAGAGTCCCATGG | | NdeI/BamHI sites of |
| Rvs | ATGATGG (SEQ ID NO: | | pET28a |
| | 86) | | |
| pHM2182 | GGTGCCGCGCGGCAGCC | BdDHSnc | In-Fusion cloning into |
| BdDHSnc | ATATGCGCGCGACGTCG | | NdeI/BamHI sites of |
| Fwd | GTCGCGGC (SEQ ID NO: | | pET28a |
| | 87) | | |
| pHM2183 | CGGAGCTCGAATTCGGA | BdDHSnc | In-Fusion cloning into |
| BdDHSnc | TCCTTAAGCTTCTACTCT | | NdeI/BamHI sites of |
| Rvs | AGATATCAAGC (SEQ ID | | pET28a |
| | NO: 88) | | |
| pHM2342 | GGTGCCGCGCGGCAGCC | SbDHS1a | In-Fusion cloning into |
| SbDHS1a | ATATGGCCATCCACGCC | | NdeI/BamHI sites of |
| Fwd | GCCGACCC (SEQ ID NO: | | pET28a |
| | 89) | | |
| pHM2343 | CGGAGCTCGAATTCGGA | SbDHS1a | In-Fusion cloning into |
| SbDHS1a | TCCTCAGAAAGCCAGTG | | NdeI/BamHI sites of |
| Rvs | GTGGCAGC (SEQ ID NO: | | pET28a |
| | 90) | | |
| pHM2344 | GGTGCCGCGCGGCAGCC | SbDHS2 | In-Fusion cloning into |
| SbDHS2 | ATATGCTCCGCGCCCGC | | NdeI/BamHI sites of |
| Fwd | GCCGTCC (SEQ ID NO: | | pET28a |
| | 91) | | |
| pHM2345 | CGGAGCTCGAATTCGGA | SbDHS2 | In-Fusion cloning into |
| SbDHS2 | TCCTCACAGACGAATGG | | NdeI/BamHI sites of |
| Rvs | AACCCAGC (SEQ ID NO: | | pET28a |
| | 92) | | |
| pHM2675 | TCACTCTGTGGTCTCAAA | BdDHS1a | In-Fusion cloning into |
| BdDHS3a | TGGCCGTGCACGCCGCC | | pAGM1287 BsaI sites as |
| GGF | G (SEQ ID NO: 93) | | Golden Gate level 0 part |
| pHM2676 | CCACTTCGTGGTCTCACG | BdDHS1a | In-Fusion cloning into |
| BdDHS3a | AACTGAAGGCCAATGGC | | pAGM1287 BsaI sites as |
| GGR | GGC (SEQ ID NO: 94) | | Golden Gate level 0 part |
| pHM2546 | TCACTCTGTGGTCTCAAA | BdDHS1b | In-Fusion cloning into |
| BdDHS3b | TGGCCGTCCACGCCGCG | | pAGM1287 BsaI sites as |
| GGF | GAGCC (SEQ ID NO: 95) | | Golden Gate level 0 part |
| pHM2547 | CCACTTCGTGGTCTCACG | BdDHS1b | In-Fusion cloning into |
| BdDHS3b | AACTGAAACCATAGGTT | | pAGM1287 BsaI sites as |
| GGF | GGCAATG (SEQ ID NO: | | Golden Gate level 0 part |
| | 96) | | |
| pHM2677 | TCACTCTGTGGTCTCAAA | BdDHS2 | In-Fusion cloning into |
| BdDHS3c | TGATCCGCGCGCACGCG | | pAGM1287 BsaI sites as |
| GGF | G (SEQ ID NO: 97) | | Golden Gate level 0 part |
| pHM2678 | CCACTTCGTGGTCTCACG | | In-Fusion cloning into |
| BdDHS3c | AACTGAGTCCCATGGAT | BdDHS2 | pAGM1287 BsaI sites as |
| GGR | GATG (SEQ ID NO: 98) | | Golden Gate level 0 part |

### Enzyme Assays

TyrA assays were conducted in a plate reader at 37° C. (Tecan Infinite M Plex, Tecan) using half-area plates (Greiner Bio-One) by tracking the conversion of NAD (P)⁺ into NAD (P) H as the increment of absorbance at 340 nm. TyrA reactions consisted of a final volume of 50 μL of 50 mM HEPES buffer pH 7.5, 50 mM KCl, 1 mM NADP⁺ (NAD⁺), and the enzyme (variable concentration, see details below). For IC₅₀ assays, tyrosine from 10×-stocks adjusted to PH˜10 with NaOH was included in the reaction mixture, as tyrosine solubility is low at neutral pH. Enzyme concentration was adjusted using TyrA desalting buffer supplemented with bovine serum albumin (BSA, protease-free powder purified by heat shock process; Fisher bioreagents), to ensure at least 3 minutes of linear reaction. For arogenate-NADP⁺ activity, the mass of enzyme was adjusted to 10 to 200 ng per reaction, depending on the specific activity of the TyrA isoform being tested. For assays using NAD⁺ and/or prephenate, the enzyme mass per reaction was scaled up to 200-1,000 ng to increase sensitivity. The reactions mixtures with the enzyme and without substrate (arogenate or prephenate) were incubated at 37° C. for 3 minutes upon the addition of the substrate. The final concentration of substrate varied depending on the experiment. For determination of the enzyme substrate, 1 mM of prephenate or arogenate was used. For Kₘ and k_{cat} determination, variable concentrations of up to 2.5 mM of arogenate were used. For IC₅₀ determination, 0.5 mM of arogenate was used.

Except when specified (FIG. 14), DHS activity was measured using a real-time method by tracking the consumption of PEP at 232 nm⁴⁹ at 37° C. in a plate reader (Tecan Infinite M Plex, Tecan) in half-area UV-transparent 96-well plates (UV-Star®, Greiner Bio-One). DHS reactions consisted of a final volume of 50 μL of 25 mM HEPES buffer pH 7.5, 2 mM MgCl₂, 3 mM DTT, the enzyme (variable mass, see details below), the effector (if tested) and the substrates (PEP, E4P). All DHS effectors tested were included in the initial reaction mixtures at a concentration of 0.5 mM, except for IC₅₀ determination for arogenate and tryptophan, in which variable concentrations were used. To ensure at least 10 minutes of linear reaction, enzyme mass per reaction was carefully adjusted to between 100 to 300 ng (depending on the specific activity of each specific isoform) using DHS storage buffer supplemented with BSA. The reaction mixtures, having the enzyme and all the other components except PEP nor E4P, were incubated for 5 minutes at room temperature to allow for DTT-mediated activation of DHS. After this, PEP (variable concentration, see below) was mixed into the reaction, and a second incubation step of 5 minutes at 37° C. was performed. The enzymatic reaction was started with the addition of E4P (variable concentration, see below). The concentrations of the substrates, PEP and E4P, were varied depending on the specific experiment. For testing potential feedback inhibitors and determination of IC₅₀, 1.5 mM PEP and 2 mM E4P was used. For calculating Kₘ and k_{cat} for PEP, a fixed concentration of 2 mM E4P and variable concentrations of PEP (up to 2 mM) were used. For calculating EC₅₀ and k_{cat} for E4P, a fixed concentration of 1.5 mM PEP and variable concentrations of E4P (up to 3 mM) were used.

For DHS effector molecules overlapping with PEP absorbance in the UV range, PEP-based quantification of DHS activity was contrasted by a final-point quantification of the reaction product DAHP by UHPLC-MS. The DHS assay for UHPLC-MS quantification was set up using the same settings as described in the previous paragraph for the UV-based DHS assay, which guaranteed at least 10 minutes of reaction linearity. After a 10-minute incubation, 20 μL of the reactions (out of a total volume of 50 μL) were mixed into 80 μL of methanol, vortexed and transferred to vials for injection. Analysis of DAHP by UHPLC-MS was conducted using the same chromatographic settings as described for the UHPLC-MS analysis of soluble metabolites and compared with an authentic DAHP standard (Sta. Cruz biotechnology, cat. no. sc-216432).

Kinetic parameters of both TyrA and DHS enzymes were determined in MS-Excel using the Solver add-in function. Arogenate was prepared by enzymatic conversion from prephenate (Prephenate Barium salt, Sigma-Aldrich) as previously described⁶⁶.

Transient Expression Experiments in Nicotiana benthamiana

Agrobacterium tumefaciens strain GV3101 transformed with the plant expression constructs were grown at 28° C. for 24 to 36 hours in 10 mL of LB liquid media containing the corresponding antibiotics. The saturated cultures were spun down at 3,000 g for 5 minutes at room temperature and washed twice with 3 mL of induction media (IM; 10 mM MES [2-(N-morpholino) ethanesulfonic acid] buffer pH 5.6, 0.5% glucose, 2 mM NaH₂PO₄, 20 mM NH₄Cl, 1 mM MgSO₄, 2 mM KCl, 0.1 mM CaCl₂), 0.01 mM FeSO₄, and 0.2 mM acetosyringone). After washing, bacteria cultures were incubated in IM for 2 to 3 hours at room temperature in the dark, pelleted at 3000 g for 5 minutes, and resuspended into 3 mL of 10 mM MES buffer pH 5.6 with 0.2 mM acetosyringone. OD₆₀₀ₙₘ was adjusted to a final density of 0.25 units for pAGM4673::TyrA (FIG. 3F) and pICH47822::DHS (FIG. 5A) infiltration, or 0.5 units for pAGM4673::TyrA-DHS co-expression constructs (FIG. 5B) using 10 mM MES buffer pH 5.6 with 0.2 mM acetosyringone. For infiltration of pICH47822::DHS constructs, the Agrobacterium suspensions were adjusted to OD₆₀₀ₙₘ=0.5 and mixed with an equal volume of a suspension of an Agrobacterium clone transformed with pICH4780::p19 under control of the Arabidopsis ubiquitin ligase promoter adjusted to OD₆₀₀ₙₘ=0.5, resulting in a final mixture of 0.25 OD₆₀₀ₙₘ units for each construct. The inclusion of the p19 gene silencing suppressor was found to improve transient expression of grass DHS genes in Nicotiana benthamiana. Nicotiana benthamiana plants of around 4-weeks-old were infiltrated close to the end of the light period into four different spots per plant, distributed into two leaves at two infiltrations per leaf, with each individual spot corresponding with a different construct/treatment. In total, each construct was infiltrated as 5 or 6 independent replicates into different plants following a randomized pattern. Samples consisting of the infiltrated leaf limbs, without the main veins, were harvested at ˜72 hours post infiltration and subjected to HPLC or UHPLC-MS analysis (see details below).

¹³CO₂ Feeding

Brachypodium, Setaria, and Arabidopsis plants were grown in 2.5×2.5-inch pots and randomly distributed into a plexiglass labeling chamber of approximately 32 liters of total volume. The artificial air mixture containing 79% N₂, 21% O₂, and 0.040% (400 ppm) ¹³CO₂ was pumped at a normal flow rate of 2 liters per minute. The air flow was connected 15 minutes before the beginning of the light period. For sampling, the air flow was interrupted, and the plant samples (entire plants for the experiment represented in FIG. 1; fully expanded leaves and stem tissue for the experiment represented in FIG. 2) were harvested and frozen immediately in liquid nitrogen. Feeding was resumed by reconnecting the air flow at 10 liters per minute with no ¹³CO₂ included in the mixture to quickly purge atmospheric ¹²CO₂. After 5 minutes, the flow rate was resumed at 2 liters per minute, ˜400 ppm ¹³CO₂, and kept constant until the next sampling time. Light intensity and temperature during the experiment were ˜ 100 μE and 22° C., respectively.

### UHPLC-MS/MS Analysis of Metabolites

Around 30-40 mg of pulverized frozen plant tissue were resuspended into 400 μL of chloroform:methanol (1:2) for ˜1 hour with regular vortexing, followed by centrifugation at 20,000 g for 5 minutes at room temperature. The whole supernatant was transferred to a fresh tube, mixed with 125 μL of chloroform, 300 μL of water, and spun down at 15,000 g for 5 minutes for phase separation. The upper, aqueous phase was recovered and dried down for 4 hours to overnight in a speed-vac at 40° C. The dried pellets were resuspended into 100 μL of methanol 80%, spun down at 20,000 g for 5 minutes, and the supernatant transferred to vials for injection. All reagents used for the extraction were UHPLC-MS grade.

Aromatic amino acids and shikimate were detected using a Vanquish Horizon Binary UHPLC (Thermo Scientific) coupled to a Q Exactive mass spectrometer (Thermo Scientific). Two microliters of the sample were analyzed using a InfinityLab Poroshell 120 HILIC-Z column (150×2.1 mm, 2.7-μm particle size; Agilent) in a gradient of 5 mM ammonium acetate/0.2% acetic acid buffer in water (solvent A) and 5 mM ammonium acetate/0.2% acetic acid buffer in 95% acetonitrile (solvent B) at a flow rate of 0.45 mL/min and column temperature of 40° C. The phase B gradient was: 0-2 min, 94%; 2-9 min, 94-88%; 9-19 min, 88-71%; 19-20 min, 71-20%, 20-21.5 min, 20%; 21.5-22 min, 20-94%; 22-25 min, 94%. All chemicals used to prepare the mobile phases were LC-MS grade. Full MS spectra were recorded between 2 and 19 minutes using full scan in negative mode, under the following parameters: sheath gas flow rate, 55; auxiliary gas flow rate, 20; sweep gas flow rate, 2; spray voltage, 3 kV; capillary temperature, 400° C.; S-lens RF level, 50; resolution, 70,000; AGC target 3×10⁶, maximum scan time 100 ms; scan range 70-1050 m/z. Spectral data were integrated manually using Xcalibur 3.0. For ¹³C labeled plant samples, ¹³C-isotopologues were detected based on a mass increase of 1.00335 atomic mass units for each ¹³C atom. Compound abundance was calculated based on high purity standards: Amino Acid Standard H for tyrosine and phenylalanine (Thermo Scientific, cat. no. PI20088), and shikimic acid ≥99% (Millipore Sigma, cat. no. S5375).

Determination of Tyrosine Content by HPLC in Nicotiana benthamiana Extracts

The infiltrated leaf areas, excluding the midrib and major veins, were harvested at 72 hours after infiltration and frozen immediately in liquid nitrogen. Then, 15 to 25 mg of pulverized frozen plant tissue were extracted into 400 μL of 0.5% 2-amino-2-methyl-1-propanol buffer pH 10.0 in 75% ethanol, as described before⁶⁷. Plant extracts were analyzed by HPLC (Infinity 1260, Agilent, Santa Clara, CA) equipped with a Water's Atlantis T3 C18 column (3μ, 2.1×150 mm) using mobile phases of A (water with 0.1% formic acid) and B (acetonitrile with 0.1% formic acid) in a 20 min gradient of the mobile phase B: 0 to 5 min, 1% isocratic; 5 to 10 min, linear increase from 1% to 76%; 10 to 12 min, linear decrease from 76% to 1%; 12 to 20 min, 1% isocratic. A tyrosine peak was detected at the retention time of ˜3.5 minutes using fluorescence detection mode (excitation wavelength 274 nm, emission wavelength 303 nm) and quantified with an authentic tyrosine standard (Alfa Acsar, catalog number AAA1114118).

### TyrA Sequence Identification and Phylogenetic Analysis

TyrA and DHS protein sequences were downloaded from Phytozome⁶⁸ v13 using pBLAST search in the following genomes (species abbreviations between parenthesis): Amaranthus hypochondriacus v2.1 (AH), Ananas comosus v3 (Aco), Arabidopsis thaliana TAIR10 (At), Beta vulgaris EL10_1.0 (EL), Chlamydomonas reinhardtii v5.6 (Cre), Cucumis sativus v1.0 (Cucsa), Brachypodium distachyon v3.2 (Bd), Gossypium raimondii v2.1 (Gorai), Hordeum vulgare Morex v3 (HORVU), Marchantia polymorpha v3.1 (Mapoly), Medicago truncatula Mt4.0v1 (Medtr), Musa acuminata v1 (GSMUA), Oropetium thomaeum v1.0 (Oropetium), Oryza sativa v7.0 (LOC_Os), Panicum virgatum v5.1 (Pavir), Phaseolus vulgaris v2.1 (Phvul), Populus trichocarpa v4.1 (Potri), Selaginella moellendorffii v1.0 (Selmo), Setaria viridis v2.1 (Sevir), Solanum lycopersicum ITAG4.0 (Solyc), Spinacia oleracea Spov3 (Spov), Sorghum bicolor v3.1.1 (Sb), Zea mays RefGen_V4 (Zm), Zostera marina v3.1 (Zosma). Protein sequences without the putative plastid transit peptide were aligned using MUSCLE in MEGA-1169. Phylogenies were reconstructed in MEGA-11 using the Neighbor-Joining method and a site coverage cutoff was set at 90%. The tree is drawn to scale, with branch lengths in the same units as those of the evolutionary distances used to infer the phylogenetic tree. The evolutionary distances were computed using the Poisson correction method and are in the units of the number of amino acid substitutions per site. Bootstrap values were calculated based on 1000 replications.

Cloning of TyrA and DHS Genes into pET28a

Plant total RNA used for cloning was extracted from young leaf tissue using the CTAB/LiCl method⁷¹ with modifications⁷². cDNA was synthesized with SuperScript IV VILO Master Mix (Thermo Scientific) following the manufacturer's instructions.

All genes were cloned without the predicted plastid transit peptide (TargetP v2.0 server, DTU Health Tech) using specific primers listed in Table 3. TyrA1 and TyrA2 genes were directly cloned from genomic DNA, as these genes lack introns. Grass TyrAnc genes were cloned from cDNA. DHS genes from Brachypodium were cloned from cDNA. SbDHS1a and SbDHS2 were cloned from Sorghum bicolor cDNA. SbDHS1b and SbDHSnc were synthesized into the pET28a vector (GeneArt, Thermo-Fisher). All cloning PCRs were conducted using high fidelity DNA polymerase (PrimeSTAR Max DNA polymerase, Takara Bio). PCR amplicons were purified from gel using QIAquick gel extraction kit (QIAGEN) and cloned into the pET28a vector between the NdeI and BamHI sites by In-Fusion cloning (Clontech). All cloned genes were confirmed by Sanger sequencing.

### Plant Expression Constructs

For transient expression of TyrA genes of Brachypodium in Arabidopsis protoplasts, the full-length CDSs, without stop codon, were amplified by PCR from cDNA (BdTyrAnc) or genomic DNA (BdTryA1 and BdTyrA2, which lack introns) using corresponding gene-specific primers (Table 3). cDNA was prepared as described for pET28a constructs. The PCR fragments were purified from gel and inserted into the vector backbone pML94 at KpnI and NotI sites, using the In-Fusion cloning (Clontech). The constructs were confirmed by restriction digestion and Sanger sequencing.

For TyrA expression in Nicotiana benthamiana under control of CaMV 35S promoter, the TyrA genes were amplified from pET28a constructs and assembled into a modified version of the binary vector pAGM4673 (Addgene plasmid #48014, courtesy of Sylvestre Marillonnet⁷⁰ (FIG. 13) using BsaI sites introduced downstream of the CaMV 35S promoter. The plastid transit peptide from the enzyme 3-enol-pyruvyl-shikimate-3-phosphate synthase from Petunia×hybrida was used to target the TyrA proteins into the plastid⁷³. GUS, AtTyrA2 and BvTyrAα expression vectors were assembled by Dr. Ray Collier.

For the simultaneous expression of Brachypodium TyrA and DHS genes in Nicotiana benthamiana, the genes were first cloned into the level 0 backbone pAGM1287 (Addgene plasmid #47996, courtesy of Sylvestre Marillonnet⁷⁰ by In-Fusion cloning (Clontech). The level 0 modules were assembled into the level 1 binary vector pICH47831 for TyrA enzymes, or into pICH47822 for DHS enzymes (Addgene plasmids #48009 and #48010, courtesy of Sylvestre Marillonnet⁷⁰ as illustrated in FIG. 13, using the MoClo Plant Parts Kit (Addgene Kit #1000000047, courtesy of Nicola Patron⁵¹). The level 1 modules were then transferred into the level 2 binary backbone pAGM4673 (FIG. 13). All constructs were checked by restriction digestion and Sanger sequencing prior to being transformed into Agrobacterium tumefaciens GV3101. All primers used are listed in Table 3.

### Protein Expression and Purification

Recombinant proteins were produced using the E. coli strains Rosetta-2 (DE3) (Millipore Sigma) for TyrAnc enzymes, ArcticExpress (Agilent) for TyrA1 and TyrA2 enzymes, and KRX (Promega) for DHS enzymes. In all cases, starter cultures were grown overnight at 37° C., 200 rpm in 10 mL terrific broth (TB) medium containing the corresponding pET28a antibiotic (50 μg/mL kanamycin) and 0.1% glucose. The next day, flasks containing 200 or 400 mL of TB medium with 50 μg/ml kanamycin and without glucose were inoculated with a 1:100 dilution of the starter cultures and kept at 37° C., 200 rpm, until the OD_{600 nm} reached ˜0.5-0.6. For TyrAnc production in Rosetta-2, the cultures were cooled down to room temperature for ˜15 minutes, induced with 0.5 mM of isopropyl β-D-1-thiogalactopyranoside (IPTG), and kept at 22° C., 200 rpm, for 8 to 10 hours. For production of DHS proteins in KRX, the cultures were cooled down to room temperature for ˜15 minutes, induced with 0.5 mM IPTG and 0.1% rhamnose, and kept at 22° C., 200 rpm, for 16-20 hours. For production of TyrA1 and TyrA2, ArcticExpress cultures were cooled down in a mixture of water and ice for ˜10 minutes, induced with 0.5 mM IPTG and kept at 15° C., 200 rpm, for 16 to 20 hours. All cultures were pelleted at 5000 g for 10 minutes and stored at −80° C. until purification.

Frozen bacterial pellets were thawed on ice and resuspended into 2 to 4 mL of LEW buffer (Lysis-Equilibration-Washing buffer; 50 mM sodium phosphate buffer pH 8.3, 300 mM NaCl 300 mM and 10% v/v glycerol) supplemented with 1 mM PMSF and 1 mg/mL lysozyme and sonicated on ice for 5 minutes in 30 second cycles. Cell lysate was centrifuged at 15,000 g, 4° C., for 15 minutes. The supernatant was recovered, mixed with 100 μL of PureProteome Nickel Magnetic Beads (Millipore) previously washed with LEW buffer, and kept in the cold under gentle shaking for 30 minutes for binding. After that, the magnetic beads were washed twice with 1 mL of LEW buffer. Proteins were eluted with LEW buffer with 250 mM imidazole into four fractions of 100 μL each. The fraction(s) with the highest protein concentration (usually two) were combined and exchanged into the corresponding storage buffer using Sephadex G-50 resin (GE Healthcare): for TyrA proteins, 50 mM 4-(2-hydroxyethyl)-1-piperazineethanesulfonic acid (HEPES) buffer pH 7.5, 50 mM KCl, 10% glycerol, and 1 mM dithiothreitol (DTT); for DHS proteins, 50 mM HEPES buffer pH 7.5, 300 mM NaCl, and 0.2% Triton X-100. In the case of DHS proteins, keeping NaCl concentration at >150 mM in the storage buffer was found to be critical to prevent protein precipitation. Buffer-exchanged proteins were frozen immediately in liquid nitrogen and stored at −80° C. The concentration of total protein was determined using Bio-Rad Protein Assay Dye Reagent Concentrate (Bio-Rad). Purity level of the recombinant enzymes was determined in ImageJ (v1.52a) upon staining of SDS-PAGE gel with Coomassie Brilliant Blue R250. Enzymatic assays were carried out within no longer than 2 weeks of protein storage at −80° C., although we found many TyrA and DHS enzymes to be stable for longer periods (i.e., a few months) under these conditions.

### Plastid Targeting Assay in Arabidopsis Protoplasts

Localization studies for BdTyrA1, BdTyrA2, and BdTyrAnc were performed in Arabidopsis protoplasts using c-terminal fusion to EGFP. Plasmid DNA was isolated from E. coli cell cultures with the PureYield™ Plasmid Maxiprep System (Promega). Protoplasts were isolated from two-weeks-old Arabidopsis thaliana leaves, transfected with plasmid DNA, and incubated for 16 hours to allow for protein expression and maturation. Samples were analyzed by laser scanning confocal microscopy using a Zeiss LSM 780 ELYRA PS1. The light path included a 488 nm laser, a 561 nm laser, and a 488/561 dichroic mirror. Fluorescence was detected in two tracks in the range of 578 nm-696 nm and 493 nm-574 nm to record chlorophyll autofluorescence and EGFP signal, respectively. All images were captured with an LDC-Apochromat 40×/1.1 W Korr M27 objective. Images were processed using Zen software (Zeiss).

### Extraction of Plant Proteins and Western Blot

Total protein from Nicotiana benthamiana samples was extracted from ˜10 mg of pulverized frozen tissue into 75 μL of 1× denaturing protein sample buffer (60 mM Tris [tris(hydroxymethyl)aminomethane] buffer pH 6.8, 2% sodium dodecyl sulfate, 10% glycerol, 3% β-mercaptoethanol, and 0.01% bromophenol blue) by vigorous vortexing for 30 seconds and was boiled immediately at 95° C. for 7 minutes. Tubes were centrifuged at 15,000 g for 5 minutes and 5 μL of the supernatant were applied per lane to the SDS-PAGE gel. Proteins were transferred to a PVDF membrane and blocked for 1 hour in 5% skimmed milk in Tris Saline Buffer with 0.05% Tween-20 before incubation with the corresponding antibodies. HA-tagged fusion proteins were detected using an anti-HA tag monoclonal antibody conjugated to HRP at a 1:1,000 dilution (HA-Probe HRP conjugated mouse monoclonal antibody clone F-7, Sta. Cruz Biotechnology, cat. no. SC-7392). Antibody dilutions were prepared in Tris Buffer Saline with 0.05% Tween-20 and 0.5% BSA. Immunoblot signal was quantified in non-saturating conditions using ImageJ (version 1.52a) and pure recombinant BdDHS1b-3×HA as a standard, which was mixed with total protein extracts of not-infiltrated Nicotiana leaves to ensure homogenous transfer for all lanes. Independent western blot membranes were exposed in parallel to ensure quantitative results. For details about the generation of the recombinant protein standards, see the section of the Materials and Methods titled “Protein expression and purification”.

### REFERENCES

1. Maeda H A, and Dudareva N (2012) The Shikimate Pathway and Aromatic Amino Acid Biosynthesis in Plants. Annu Rev Plant Biol, 63:73-105.
2. Lynch J H, and Dudareva N (2020) Aromatic Amino Acids: A Complex Network Ripe for Future Exploration. Trends Plant Sci, 25:670-681.
3. Boerjan W, Ralph J, and Baucher M (2003) Lignin Biosynthesis. Annu Rev Plant Biol, 54:519-546.
4. Cochrane F C, Davin L B, and Lewis N G (2004) The Arabidopsis Phenylalanine Ammonia Lyase Gene Family: Kinetic Characterization of the Four PAL Isoforms. Phytochemistry, 65:1557-1564.
5. Barros J, and Dixon R A (2020) Plant Phenylalanine/Tyrosine Ammonia-Lyases. Trends Plant Sci, 25:66-79.
6. Neish A C (1961) Formation of m- and p-Coumaric Acids by Enzymatic Deamination of the Corresponding Isomers of Tyrosine. Phytochemistry, 1:1-24.
7. Young M R, Towers G H N, and Neish A C (1966) Taxonomic Distribution of Ammonia-lyases for L-Phenylalanine and L-Tyrosine in Relation to Lignification. Can J Bot, 44:341:349
8. Reid P D, Havir E A, and Marsh H V (1972) L-Phenylalanine Ammonia-Lyase (Maize). Plant Physiol, 50:480-484.
9. Rösler J, Krekel F, Amrhein N, and Schmid J (1997) Maize Phenylalanine Ammonia-lyase has Tyrosine Ammonia-Lyase Activity. Plant Physiol, 113:175-179.
10. Barros J, Serrani-Yarce J C, Chen F, Baxter D, Venables B J, and Dixon R A (2016) Role of Bifunctional Ammonia-lyase in Grass Cell Wall Biosynthesis. Nat Plants, 2:16050.
11. Jun S Y, Sattler S A, Cortez G S, Vermerris W, Sattler S E, Kang C (2018) Biochemical and Structural Analysis of Substrate Specificity of a Phenylalanine Ammonia-Lyase. Plant Physiol, 176:1452-1468.
12. Higuchi T, Ito Y, and Kawamura I (1967). p-Hydroxyphenylpropane Component of Grass Lignin and Role of Tyrosine Ammonia-Lyase in its Formation. Phytochemistry, 6:875-881.
13. Simpson J P, Olson J, Dilkes B, and Chapple C (2021) Identification of the Tyrosine- and Phenylalanine-Derived Soluble Metabolomes of Sorghum. Front Plant Sci, 12:714164.
14. Tzin V, and Galili G (2010) New Insights into the Shikimate and Aromatic Amino Acids Biosynthesis Pathways in Plants. Mol Plant, 3:956-972.
15. Yokoyama R, de Oliveira M V V, Kleven B, and Maeda H A (2021) The Entry Reaction of the Plant Shikimate Pathway is Subjected to Highly Complex Metabolite-Mediated Regulation. Plant Cell, 33:671-696.
16. Yokoyama R, de Oliveira M V V, Takeda-Kimura Y, Ishihara H, Alseekh S, Arrivault S, Kukshal V, Jez J M, Stitt M, Fernie A R, and Maeda H A (2022) Point Mutations that Boost Aromatic Amino acid Production and CO₂ Assimilation in Plants. Sci Adv, 8: eabo3416.
17. Byng G S, Whitaker R J, Flick C, and Jensen R A (1981) Enzymology of L-Tyrosine Biosynthesis in Corn (Zea mays). Phytochem, 208:1289-1292.
18. Gaines C G, Byng G S, Whitaker R J, and Jensen R A (1982) L-Tyrosine Regulation and Biosynthesis via Arogenate Dehydrogenase in Suspension-Cultured cells of Nicotiana silvestris. Planta, 1568:233-240.
19. Connelly J A, and Conn E E (1986) Tyrosine Biosynthesis in Sorghum bicolor: Isolation and Regulatory Properties of Arogenate Dehydrogenase. Z Naturforsch C, 41:69-78.
20. Rippert P, and Matringe M (2002) Purification and Kinetic Analysis of the Two Recombinant Arogenate Dehydrogenase Isoforms of Arabidopsis thaliana. Eur J Biochem, 269:4753-4761.
21. Yoo H, Shrivastava S, Lynch J H, Huang X Q, Widhalm J R, Guo L, Carter B C, Qian Y, Maeda H A, Ogas J P, Morgan J A, Marshall-Colón A, Dudareva N (2021) Overexpression of Arogenate Dehydratase Reveals an Upstream Point of Metabolic Control in Phenylalanine Biosynthesis. Plant J, 108:737-751.
22. El-Azaz J, Cánovas F M, Barcelona B, Ávila C, and de la Torre F (2022) Deregulation of Phenylalanine Biosynthesis Evolved with the Emergence of Vascular Plants. Plant Physiol, 188:134-150.
23. Herrmann, K M (1995) The Shikimate Pathway: Early Steps in the Biosynthesis of Aromatic Compounds. Plant Cell, 7:907-919.
24. Yokoyama R, Kleven B, Gupta A, Wang Y, and Maeda H A (2022) 3-Deoxy-D-Arabino-Heptulosonate 7-Phosphate Synthase as the Gatekeeper of Plant Aromatic Natural Product Biosynthesis. Curr Opin Plant Biol, 67:102219.
25. Verdonk J C, Haring M A, van Tunen A J, and Schuurink R C (2005) ODORANT1 Regulates Fragrance Biosynthesis in Petunia Flowers. Plant Cell, 17:1612-1624.
26. Maeda H A, Yoo H, and Dudareva N (2011) Prephenate Aminotransferase Directs Plant Phenylalanine Biosynthesis via Arogenate. Nat Chem Biol, 7:19-21.
27. Dal Cin V, Tieman D M, Tohge T, McQuinn R, de Vos R C, Osorio S, Schmelz E A, Taylor M G, Smits-Kroon M T, Schuurink R C, Haring M A, Giovannoni J, Fernie A R, and Klee H J (2011) Identification of Genes in the Phenylalanine Metabolic Pathway by Ectopic Expression of a MYB Transcription Factor in Tomato Fruit. Plant Cell, 23:2738-2753.
28. Corea O R, Ki C, Cardenas C L, Kim S J, Brewer S E, Patten A M, Davin L B, and Lewis N G (2012) Arogenate Dehydratase Isoenzymes Profoundly and Differentially Modulate Carbon Flux into Lignins. J Biol Chem, 287:11446-11459.
29. Obayashi T, Aoki Y, Tadaka S, Kagaya Y, and Kinoshita K (2018) ATTED-II in 2018: A Plant Coexpression Database Based on Investigation of the Statistical Property of the Mutual Rank Index. Plant Cell Physiol, 59: e3.
30. Voll L, Häusler R E, Hecker R, Weber A, Weissenböck G, Fiene G, Waffenschmidt S, and Flügge U-I (2003) The Phenotype of the Arabidopsis cue1 Mutant is not Simply Caused by a General Restriction of the Shikimate Pathway. Plant J, 36:301-317.
31. Voll L M, Allaire E E, Fiene G, and Weber A P (2004) The Arabidopsis Phenylalanine Insensitive Growth Mutant Exhibits a Deregulated Amino Acid Metabolism. Plant Physiol, 136:3058-3069.
32. Mancla N, Oliva M, Ovadia R, Sikron-Persi N, Ayenew B, Fait A, Galili G, Perl A, Weiss D, and Oren-Shamir M (2015) Phenylalanine and Tyrosine Levels are Rate-Limiting Factors in Production of Health Promoting Metabolites in Vitis vinifera cv. Gamay Red Cell Suspension. Front Plant Sci, 6:538.
33. Qian Y, Lynch J H, Guo L, Rhodes D, Morgan J A, and Dudareva N (2019) Completion of the Cytosolic Post-Chorismate Phenylalanine Biosynthetic Pathway in Plants. Nat Commun, 10:15.
35. Higuchi T, and Shimada M (1969) Metabolism of Phenylalanine and Tyrosine During Lignification of Bamboos. Phytochemistry, 8:1185-1192.
36. Robinson P W, and Hodges C F (1977) Effect of Nitrogen Fertilization on Free Amino Acid and Soluble Sugar Content of Poa pratensis and on Infection and Disease Severity by Drechslera sorokiniana. Phytopathology, 67:1239-1244.
37. Seebauer J R, Moose S P, Fabbri B J, Crossland L D, and Below F E (2004) Amino Acid Metabolism in Maize Earshoots. Implications for Assimilate Preconditioning and Nitrogen Signaling. Plant Physiol, 136:4326-4334.
38. de Souza L P, Szecówka M, Fernie A R, and Tohge T (2017) ¹³CO₂ Labeling and Mass Spectral Analysis of Photorespiration. In: Fernie A, Bauwe H, and Weber A (eds) Photorespiration. Methods in Molecular Biology, vol 1653. Humana Press, New York, N Y.
39. Schenck C A, and Maeda H A (2018) Tyrosine Biosynthesis, Metabolism, and Catabolismin Plants. Phytochemistry, 149:82-102.
40. Schenck C A, Chen S, Siehl D L, and Maeda H A (2015) Non-plastidic, Tyrosine-Insensitive Prephenate Dehydrogenases from Legumes. Nat Chem Biol, 11:52-57.
41. Ruprecht C, Mendrinna A, Tohge T, Sampathkumar A, Klie S, Fernie A R, Nikoloski Z, Persson S. and Mutwil M. (2016) FamNet: a Framework to Identify Multiplied Modules Driving Pathway Expansion in Plants. Plant Physiol, 170:1878-1894.
42. Makita Y, Shimada S, Kawashima M, Kondou-Kuriyama T, Toyoda T, and Matsui M (2015) MOROKOSHI: Transcriptome Database in Sorghum bicolor. Plant Cell Physiol, 56: e6.
43. Kellogg E A (2001) Evolutionary History of the Grasses. Plant Physiol, 125:1198-1205.
44. Soreng R J, Peterson P M, Romaschenko K, Davidse G, Zuloaga F O, Judziewicz E J, Filgueiras T S, Davis J I, and Morrone O (2015) A Worldwide Phylogenetic Classification of the Poaceae (Gramineae) J Syst Evol, 53:117-137.
45. Lopez-Nieves S, Yang Y, Timoneda A, Wang M, Feng T, Smith S A, Brockington S F, and Maeda H A (2018) Relaxation of Tyrosine Pathway Regulation Underlies the Evolution of Betalain Pigmentation in Caryophyllales. New Phytol, 217:896-908.
46. Schenck C A, Holland C K, Schneider M R, Men Y, Lee S G, Jez J M, and Maeda H A (2017) Molecular Basis of the Evolution of Alternative Tyrosine Biosynthetic Routes in Plants. Nat Chem Biol, 13:1029-1035.
47. Hudson G S, Wong V, and Davidson B E (1984) Chorismate Mutase/Prephenate Dehydrogenase from Escherichia coli K12: Purification, Characterization, and Identification of a Reactive Cysteine. Biochemistry, 23:6240-6249.
48. Lütke-Eversloh T, and Stephanopoulos G (1985) Feedback Inhibition of Chorismate Mutase/Prephenate Dehydrogenase (TyrA) of Escherichia coli: Generation and Characterization of Tyrosine-Insensitive Mutants. Appl Environ Microbiol, 71:7224-7228.
49. Schoner R, and Herrmann K M (1976) 3-Deoxy-D-Arabino-Heptulosonate 7-Phosphate Synthase. Purification, Properties, and Kinetics of the Tyrosine-Sensitive Isoenzyme from Escherichia coli. J Biol Chem, 251:5440-5447
50. Zamir L O, Jense R A, Arison B H, Douglas A W, Albers-Schoenberg G, and Bowen J R (1980) Structure of Arogenate (pretyrosine), an Amino Acid Intermediate of Aromatic Biosynthesis. J Am Chem Soc, 102:4499-4504
51. Webby C J, Jiao W, Hutton R D, Blackmore N J, Baker H M, Baker E N, Jameson G B, and Parker E J (2010) Synergistic Allostery, a Sophisticated Regulatory Network for the Control of Aromatic Amino Acid Biosynthesis in Mycobacterium tuberculosis. J Biol Chem, 285:30567-30576.
52. Engler C, Youles M, Gruetzner R, Ehnert T-M, Werner S, Jones J D G, Patron N J, and Marillonnet S (2014) A Golden Gate Modular Cloning Toolbox for Plants. ACS Synthetic Biology, 3:839-843.
53. Vogt T (2010) Phenylpropanoid Biosynthesis. Mol Plant, 3:2-20.
54. Maeda H A, and Fernie, A R (2021) Evolutionary History of Plant Metabolism. Annu Rev Plant Biol, 72:185-216.
55. Maeda, H (2016) Lignin biosynthesis: Tyrosine Shortcut in Grasses. Nat Plants 2, 16080.
56. Waese J, Fan J, Pasha A, Yu H, Fucile G, Shi R, Cumming M, Kelley L A, Sternberg M J, Krishnakumar V, Ferlanti E, Miller J, Town C, Stuerzlinger W, and Provart N J (2017) ePlant: Visualizing and Exploring Multiple Levels of Data for Hypothesis Generation in Plant Biology. Plant Cell, 29:1806-1821.
57. de Oliveira M V V, Jin X, Chen X, Griffith D, Batchu S, and Maeda H A (2019) Imbalance of Tyrosine by Modulating TyrA Arogenate Dehydrogenases Impacts Growth and Development of Arabidopsis thaliana. Plant J, 97:901-922.
58. Lopez-Nieves S, El-Azaz J, Men Y, Holland C K, Feng T, Brockington S F, Jez J M, and Maeda H A (2022) Two Independently Evolved Natural Mutations Additively Deregulate TyrA Enzymes and Boost Tyrosine Production in planta. Plant J, 109:844-855.
59. Tzin V, Malitsky S, Zvi M M B, Bedair M, Sumner L, Aharoni A, and Galili G (2012) Expression of a Bacterial Feedback-Insensitive 3-Deoxy-D-Arabino-Heptulosonate 7-Phosphate Synthase of the Shikimate Pathway in Arabidopsis Elucidates Potential Metabolic Bottlenecks Between Primary and Secondary Metabolism. New Phytol, 194:430-439.
60. Oliva M, Ovadia R, Perl A, Bar E, Lewinsohn E, Galili G, and Oren-Shamir M (2015) Enhanced Formation of Aromatic Amino Acids Increases Fragrance without Affecting Flower Longevity or Pigmentation in Petunia×hybrida. Plant Biotechnol J, 13:125-136.
61. Oliva M, Guy A, Galili G, Dor E, Schweitzer R, Amir R, and Hacham Y (2021) Enhanced Production of Aromatic Amino Acids in Tobacco Plants Leads to Increased Phenylpropanoid Metabolites and Tolerance to Stresses. Front Plant Sci, 11:604349.
62. Tohge T, Watanabe M, Hoefgen R, and Fernie A R (2013) Shikimate and Phenylalanine Biosynthesis in the Green Lineage. Front Plant Sci, 4:62.
63. Graziana A, and Boudet A M (1980) 3-Deoxy-D-Arabino-Heptulosonate 7-Phosphate Synthase from Zea mays: General Properties and Regulation by Tryptophan. Plant Cell Physiol, 21:793-802.
64. Jung E, Zamir L O and Jensen R A (1986) Chloroplasts of Higher Plants Synthesize L-Phenylalanine via L-Arogenate. PNAS, 83:7231-7235.
65. Ruijter J M, Ramakers C, Hoogaars W H M, Karlen Y, Bakker O, van den Hoff M J B, and Moorman A F M (2009) Amplification Efficiency: Linking Baseline and Bias in the Analysis of Quantitative PCR data. Nucleic Acids Res, 37: e45.
66. Hong S Y, Seo P J, Yang M S, Xiang F, and Park C M (2008) Exploring Valid Reference Genes for Gene Expression Studies in Brachypodium distachyon by Real-Time PCR. BMC Plant Biol, 8:112.
67. Maeda H A, Shasany A K, Schnepp J, Orlova I, Taguchi G, Cooper B R, Rhodes D, Pichersky E, and Dudareva N (2010) RNAi Suppression of Arogenate Dehydratase 1 Reveals that Phenylalanine is Synthesized Predominantly via the Arogenate Pathway in Petunia Petals. Plant Cell, 22:832-849.
68. Goodstein D M, Shu S, Howson R, Neupane R, Hayes R D, Fazo J, Mitros T, Dirks W, Hellsten U, Putnam N, and Rokhsar D S (2012) Phytozome: a Comparative Platform for Green Plant Genomics. Nucleic Acids Res, 40: D1178-D1186.
69. Tamura K, Stecher G, and Kumar S (2021) MEGA11: Molecular Evolutionary Genetics Analysis version 11. Mol Biol Evol, 38:3022-3027
70. Weber E, Engler C, Gruetzner R, Werner S, Marillonnet S (2011) A Modular Cloning System for Standardized Assembly of Multigene Constructs. PLOS One, 6: e16765.
71. Liao Z, Chen M, Guo L, Gong Y, Tang F, Sun X, and Tang K (2004) Rapid Isolation of High-Quality total RNA from Taxus and Ginkgo. Pre Biochem Biotechnol, 34:209-214.
72. Canales J, Rueda-López M, Craven-Bartle B, Avila C, and Cánovas F M (2012) Novel Insights into Regulation of Asparagine Synthetase in Conifers. Front Plant Sci, 3:100.
73. Della-Cioppa G, Bauer S C, Klein B K, Shah D M, Fraley R T, and Kishore G M (1986) Translocation of the Precursor of 5-Enolpyruvylshikimate-3-Phosphate Synthase into Chloroplasts of Higher Plants in vitro. Proc Natl Acad Sci USA, 83:6873-6877.

### Example 2

In the following example, the inventors demonstrate that transiently expressing DHS1b enzymes from three additional grass species (i.e., Oryza sativa, Sorghum bicolor, and Setaria viridis) in Nicotiana benthamiana leaves increases the production of aromatic amino acids and their precursors to varying degrees (FIG. 21, FIG. 22).

To determine whether the grass DHS1b gene encodes a deregulated DHS in other grass species besides Brachypodium distachyon, BdDHS1b orthologs from Oryza sativa (OsDHS1b) and Sorghum bicolor (SbDHS1b) were cloned into the Golden Gate plant expression vector pICH47822 under control of the RuBisCO small subunit 3B promoter from Arabidopsis thaliana.⁵² A 3× human influenza hemagglutinin tag was fused in frame to the C-terminus of the DHS genes to confirm the production of heterologous DHS protein.

Agrobacterium tumefaciens (strain GV3101) clones transformed with the OsDHS1b and SbDHS1b plant expression constructs were infiltrated into the leaves of Nicotiana benthamiana side-by-side with Agrobacterium clones expressing BdDHS1a, BdDHS1b, and the fluorescent protein YPet (negative control). The levels of phenylalanine, tyrosine, tryptophan, and three of their biosynthetic intermediates (shikimate, prephenate, and arogenate) were determined at 72 hours post-infiltration by liquid chromatography coupled to mass spectrometry (LCMS). The results of this analysis showed that, as for BdDHS1b, the engineered plants expressing OsDHS1b had increased levels of all measured metabolites except for tryptophan (FIG. 21). On average, expression of BdDHS1b or OsDHS1b increased phenylalanine, tyrosine, and shikimate levels by 5 to 20-times compared to the YPet negative control (FIG. 21), whereas prephenate and arogenate rose to ˜100-times the control (FIG. 21). In contrast, expression of SbDHS1b had little or no significant effect on the levels of aromatic amino acid levels and their precursor molecules (FIG. 21). Based on these findings, BdDHS1b and OsDHS1b, but not SbDHS1b, encode DHS enzymes that can boost the accumulation of aromatic amino acids in planta upon heterologous expression.

To further explore the distribution of deregulated DHS activity in grasses, this experimental approach was extended to the four members of the grass DHS gene family (DHS1a, DHS1b, DHS2, and DHSnc) (FIG. 4A) from four different grass species: Brachypodium distachyon (Bd), Oryza sativa (Os), Sorghum bicolor (Sb), and Setaria viridis (Sv). These 16 genes, in addition of the aforementioned YPet-expressing negative control, were transiently expressed side-by-side in the leaves of Nicotiana benthamiana by the Agrobacterium tumefaciens infiltration method. LCMS was used to determine the levels of the three aromatic amino acids and their biosynthetic intermediates at 72 hours post-infiltration. This experiment showed that BdDHS1b caused the highest increase in the levels of these metabolites, followed by (from highest to lowest effect) OsDHS1b, SvDHS1b, and SbDHS1b (FIG. 22). Overall, little or no significant effect was observed upon expression of any of the other three grass DHS isoforms (i.e., DHS1a, DHS2, and DHSnc) (FIG. 22). Hence, DHS1b enzymes from different grass species have a varied impact on aromatic amino acid production in planta.

What is claimed:

1. A cell engineered to express or overexpress an enzyme selected from:
a) a 3-deoxy-D-arabino-heptulosonate 7-phosphate synthase 1b (DHS1b) enzyme comprising SEQ ID NO: 3 (BdDHS1b), SEQ ID NO: 19 (OsDHS1b), SEQ ID NO: 27 (SvDHS1b) or a DHS1b enzyme having at least 95% identity to one of SEQ ID NOs: 3, 19 or 27; or
b) a noncanonical TyrA enzyme comprising SEQ ID NO: 37 (BdTyrAnc), SEQ ID NO: 43 (SbTyrAnc) or a TyrA enzyme having at least 95% identity to SEQ ID NO: 37 or 43;
wherein (i) the cell is engineered to express an enzyme that is not native to the cell, or (ii) the cell is engineered to overexpress an enzyme that is native to the cell as compared to a control cell.

2. The cell of claim 1, wherein the cell is engineered to express or overexpress both:
a) the DHS1b enzyme; and
b) a TyrA enzyme selected from the group consisting of SEQ ID NO: 33 (BdTyrA1), SEQ ID NO: 35 (BdTyrA2), SEQ ID NO: 37 (BdTyrAnc), SEQ ID NO: 39 (SbTyrA1), SEQ ID NO: 41 (SbTyrA2), SEQ ID NO: 43 (SbTyrAnc) and an enzyme having at least 95% sequence identity to one of SEQ ID NOs: 33, 35, 37, 39, 41 and 43.

3. The cell of claim 1, wherein the cell comprises an exogenous nucleic acid encoding the enzyme.

4. The cell of claim 3, wherein the exogenous nucleic acid is inserted in the genome of the cell.

5. The cell of claim 1, wherein the cell is a plant cell.

6. A plant comprising the cell of claim 5.

7. The plant of claim 6, wherein the plant is a tomato plant, tobacco plant, soybean plant, cotton plant, poplar plant, sorghum plant, corn plant, beet plant, mung bean plant, opium poppy plant, alfalfa plant, wheat plant, barley plant, millet plant, oat plant, rye plant, rapeseed plant, miscanthus plant, or grass plant.

8. The plant of claim 7, wherein the grass plant is a Sorghum bicolor plant, Oryza sativa plant, or Brachypodium distachyon plant.

9. The plant of claim 6, wherein the quantity of aromatic amino acids produced by the plant is greater than the quantity produced by a control plant.

10. A method for increasing production of one or more aromatic amino acid or derivative thereof in a cell, the method comprising engineering the cell to express or overexpress an enzyme selected from:
c) a DHS1b enzyme comprising SEQ ID NO: 3 (BdDHS1b), SEQ ID NO: 19 (OsDHS1b), SEQ ID NO: 27 (SvDHS1b) or a DHS1b enzyme having at least 95% identity to one of SEQ ID NOs: 3, 19 or 27; or
d) a noncanonical TyrA enzyme comprising SEQ ID NO: 37 (BdTyrAnc), SEQ ID NO: 43 (SbTyrAnc), or a TyrA enzyme having at least 95% identity to SEQ ID NO: 37 or 43;
wherein (i) the cell is engineered to express an enzyme that is not native to the cell, or (ii) the cell is engineered to overexpress an enzyme that is native to the cell as compared to a control cell.

11. The method of claim 10, wherein the cell is engineered to express or overexpress both:
a) the DHS1b enzyme; and
b) a TyrA enzyme selected from the group consisting of SEQ ID NO: 33 (BdTyrA1), SEQ ID NO: 35 (BdTyrA2), SEQ ID NO: 37 (BdTyrAnc), SEQ ID NO: 39 (SbTyrA1), SEQ ID NO: 41 (SbTyrA2), SEQ ID NO: 43 (SbTyrAnc) and an enzyme having at least 95% sequence identity to one of SEQ ID NOs: 33, 35, 37, 39, 41 and 43.

12. The method of claim 10, wherein the cell is engineered by introducing an exogenous nucleic acid encoding the enzyme into the cell.

13. The method of claim 12, wherein the exogenous nucleic acid is introduced via insertion into the genome of the cell.

14. The method of claim 10, wherein the cell is a plant cell.

15. The method of claim 14, wherein the plant cell is part of a plant.

16. The method of claim 15, wherein the plant is a tomato plant, tobacco plant, soybean plant, cotton plant, poplar plant, sorghum plant, corn plant, beet plant, mung bean plant, opium poppy plant, alfalfa plant, wheat plant, barley plant, millet plant, oat plant, rye plant, rapeseed plant, miscanthus plant, or grass plant.

17. The method of claim 16, wherein the grass plant is a Sorghum bicolor plant, Oryza sativa plant, or Brachypodium distachyon plant.

18. The method of claim 10 further comprising purifying aromatic amino acids or derivatives thereof from the cell.

19. A method for producing aromatic amino acids or derivatives thereof, the method comprising:
a) growing the plant of claim 6; and
b) purifying aromatic amino acids or derivatives thereof produced by the plant.

20. A method for sequestering carbon dioxide, the method comprising growing the plant of claim 6.


SEQUENCE LISTING (98 sequences)

SEQ ID NO: 1 - protein, 537 aa, Brachypodium distachyon
MALAINSAAAAAAAALSNGGAAQPPRPRAGLLPMKRRGSISAVHAADPARGTGSVPAAAKTSSPTVAPEAAASPAASWKVDSWKAKKALQLPEYPSKEELDTVLQTIETFPPIVFAGEARHLEERLAEAAMGRAFVLQGGDCAESFKEFNANNIRDTFRVLLQMGAVLMFGGQVPVVKVGRMAGQFAKPRSDNLEERDGVKLPSYRGDNVNGDAFDVKSRTPDPERMIRAYAQSVATLNLLRAFATGGYAAMQRVIQWNLDFMDHNEQGDRYRELAHRVDEALGFMTAAGLGIDHPIMTTTDFWTSHECLLLPYEQALTREDSTSGLFYDCSAHMLWVGERTRQLDGAHVEFLRGIANPLGIKVSDKMNPAELVKLIEILNPSNKPGRITIITRMGAENMRVKLPHLIRAVRNSGQIVTWITDPMHGNTIKAPCGLKTRPFDSIMNEVRAFFDVHDQEGSHPGGIHLEMTGQNVTECIGGSRTVTFDDLGDRYHTHCDPRLNASQSLELAFIIAERLRKRRMQSGLTNNLPLPPLAF

SEQ ID NO: 2 - DNA, 1614 bp, Brachypodium distachyon
  source 1..1614: mol_type = other DNA
atggcgctcgccatcaactccgccgccgccgcagcggccgccgccctctccaacggcggggcagcccagccgccgcgccccagagcggggttactcccgatgaagaggcggggcagcatctctgccgtgcacgccgccgaccccgccaggggcaccgggtccgtcccggcggccgcgaagacctcctccccgacggtggcgcccgaggcggcggcttctccggcggcgagctggaaggtcgacagctggaaggcgaagaaggccctgcagctgcccgagtacccgagcaaggaggagctcgacaccgtgctgcagaccatcgagacgttcccgcccatcgtgttcgccggggaggcgcgccacctcgaggagcgcctcgccgaggccgccatgggccgggctttcgtcctccagggcggcgactgcgccgagagcttcaaggagttcaacgccaacaacatccgtgacaccttccgtgtcctcctccagatgggcgccgtcctcatgttcggcggccaggtgcccgtcgtcaaggtaggtaggatggctggccagttcgccaagcctaggtccgacaacctcgaggagagggacggggttaagctgcccagttacaggggagacaacgtcaacggcgacgccttcgacgtcaagagccgcaccccggaccctgagaggatgatcagggcctatgcccagtcggtggcgacgctcaacttgctccgcgcgttcgccacaggaggctacgctgctatgcagcgggttatccagtggaaccttgatttcatggatcacaacgagcagggagacaggtaccgtgagttggcacatagggtggatgaggcccttggcttcatgactgcagcagggctggggatcgatcatccgataatgacaactactgacttctggacatcccatgagtgtctcctcttgccatacgagcaggctcttacccgtgaggactccacaagtggccttttctatgactgctcggcacacatgctgtgggttggtgagcgcacccggcaacttgatggggctcatgttgagttcctccgtggtattgcgaaccctcttggcattaaggtgagtgataaaatgaaccctgctgaattggtgaagctgattgagattttaaacccttcaaacaagccgggaagaataaccataattacaaggatgggtgctgagaacatgagggtgaagttgcctcatcttatccgtgctgtccgcaattctggacagattgttacatggattactgatcccatgcatgggaacaccatcaaggccccatgtggtctgaagactcgtccatttgattccattatgaatgaggtgcgtgcgttcttcgacgtgcatgaccaagaaggaagccacccaggaggtatccacctcgagatgaccggacagaatgtaaccgagtgcatcggtgggtcacggaccgtgaccttcgatgacctgggtgaccgctaccacactcactgcgacccaaggctgaatgcttcccaatctctggagctcgccttcatcatcgccgagagactcaggaagaggaggatgcagtcagggctcacaaacaacctgccactgccgccattggccttctaa

SEQ ID NO: 3 - protein, 542 aa, Brachypodium distachyon
MALATNHAAAAISSGAAAPQPRRAPSFLPLKRRTICAVHAAEPSKSAAAAPAAAKTSSPSVAPEKSAIPDPKPEAPAVPAKWTVDSWRAKKALQLPEYPNAAELESALKTIEAFPPIVFAGEARHLEERLADAAMGRAFLLQGGDCAESFKEFNGNNIRDTFRVLLQMSAVLTFGGQMPVIKVGRMAGQFAKPRSDSFEVRDGVKLPSYRGDNINGDAFNEKSRIPDPQRMIRAYTQSAATLNLLRAFAMGGYAAMQRVTQWNLDFTENSEQGDRYRELAHRVDEALGFMSAAGLTLDHPVMSSTEFWTSHECLLLPYEQALTRQDSTSGLFYDCSAHMLWVGERTRQLDGAHVEFLRGVANPLGIKVSDKMNPADLVKLIDILNPTNKPGRITIITRMGAENMRVKLPHLIRAVRHAGQIVTWITDPMHGNTIKAPCGLKTRPFDSILAEVRAFFDVHEQEGSHAGGVHLEMTGQNVTECIGGSRTVTFDDLGDRYHTHCDPRLNASQSLELSFIIAEKLRKRRIRSSKLNSVLPLPTYGF

SEQ ID NO: 4 - DNA, 1629 bp, Brachypodium distachyon
  source 1..1629: mol_type = other DNA
atggcactcgccaccaaccacgccgccgccgccatatcgtccggagccgccgcccctcagccccgccgcgccccttcgttcctcccgctgaagcgccgcacaatctgcgccgtccacgccgcggagccctcgaagagcgccgccgccgccccggccgcggcgaagacctcttcgccgtcggtggcgccggagaagtccgcgataccggacccgaaaccggaggcgcccgcggtgccagcaaagtggacggtggacagctggagggcgaagaaggctcttcagctgccggagtaccccaacgccgcggagctggagtcggccctcaagacgatcgaggcgttcccgccgatcgtcttcgccggggaggcgcggcacctggaggagcgcctcgccgacgccgccatgggccgggccttccttctccaaggaggcgactgcgccgagagcttcaaggagttcaacggcaacaatatccgcgataccttccgcgtcctgcttcagatgtctgccgtcctcacctttggcggtcaaatgcccgtcatcaaggttgggagaatggccggccaattcgcaaagccgaggtcggattcgttcgaggtaagggatggggtgaagctgccgagctaccgaggggacaacatcaacggtgatgctttcaacgaaaagagccgcatccccgatccccagaggatgatcagggcctacacccagtcggccgcgacactcaacctcctccgcgcgttcgccatgggagggtacgctgccatgcaacgggtcacccagtggaacctcgatttcactgaaaacagcgagcagggcgacaggtaccgagaattggcacacagggttgatgaagcccttggcttcatgtctgcagctgggctaacattggatcaccctgttatgtcgagtactgaattctggacctctcatgagtgcctactcctaccgtatgagcaagctttgacccgccaggactcgacctctggtcttttctatgactgttctgcccacatgctttgggtcggtgaacgcactcgccagctcgatggtgctcatgttgagttcctcaggggtgttgctaaccctcttggtatcaaggtgagtgacaaaatgaaccctgctgacttggtaaaactgattgacatattgaacccaacaaacaagcctgggagaattaccatcattacaagaatgggggcggagaacatgagggtcaagttacctcatcttatccgtgctgtccgccatgctggtcaaattgtcacctggatcactgatcccatgcatgggaacactatcaaggctccttgcggtctgaagacacgcccctttgactccattctggctgaggtcagggcgttcttcgatgttcatgagcaagaaggtagccacgcaggaggcgtccaccttgagatgactgggcagaatgtgacagagtgcattggtggatcccgaaccgtgaccttcgatgacctgggcgaccgctaccacacccactgtgacccgaggctgaatgcatcccagtctctggagctctccttcatcattgctgagaagctgaggaagaggaggatccgctcgtcgaagcttaacagcgtcttgccattgccaacctatggtttctga

SEQ ID NO: 5 - protein, 503 aa, Brachypodium distachyon
MPLAPPAAAVRNPLLPSPALAPARRGGLIRAHAVRAAPSQWAPGSWRARPALQQPEYPDKAGLDEVLRTVESFPPIVFAGEARKLEERLADAALGRAFLLQGGDCAESFKEFNANNIRDTFRVLLQMSVVLMFGGQMPIIKVGRMAGQFAKPRSDGFEERDGVKLPSYRGDNINGDVFDEKSRVPDPQRMIRAYSQSAATLNLLRAFATGGYAAMQRVTQWNLDFTEHCEQGDRYMELAHRVDEALGFMSAAGLTVDHPIMTTTEFWTSHECLLLPYEQALTREDSTSGLYYDCSAHFLWAGERTRQLDGAHVEFLRGIANPLGIKVSDKMDPKELVKLIDILNPENRPGRITIITRMGPENMRVKLPHLIRAVRGAGQIVTWVTDPMHGNTMKAPCGLKTRSFDKILAEVRAFFDVHEQEGSHPGGVHLEMTGQNVTECIGGSRTVTFDDLSSRYHTHCDPRLNASQSLELVFIIAERLRRRRAASWALDNQPGTIPSSMGL

SEQ ID NO: 6 - DNA, 1512 bp, Brachypodium distachyon
  source 1..1512: mol_type = other DNA
atgcccctcgcgccacccgccgccgccgtgcgcaacccactcctcccttccccggcgctcgccccggcgcggcgcggcgggctcatccgcgcgcacgcggtgcgcgcggcgccgagccagtgggcgccggggagctggcgggcgcggcctgcgctgcagcagccggagtacccggacaaggccgggctggacgaggtgctgcggacggtggagtcgttcccgccgatcgtcttcgcaggggaggcgcgcaaactggaggagcggctcgcggatgccgctctcggtcgcgcgttccttctccagggcggcgactgcgccgagagcttcaaggagttcaacgccaacaacatccgggacaccttccgtgtcctcctgcagatgtccgtcgtgctcatgttcggtggacagatgcctataatcaaggtaggaagaatggcaggtcaatttgcaaagccaaggtcagatggttttgaagagagggatggagtgaagttgccaagctacagaggagacaatatcaatggggatgtatttgatgagaagtcaagagtgccagatccacagcgcatgatcagggcatactcacagtctgcagcaacactgaatttgctgcgggcatttgccacaggaggttatgctgcaatgcagagggtaacacagtggaaccttgacttcacagagcattgcgaacagggtgataggtacatggagttggctcatcgagttgacgaggctttggggttcatgtcagctgctgggctcactgtagatcacccaataatgacaacaacagaattctggacatcacatgaatgccttcttcttccctacgagcaggcacttactcgtgaggattccacatctggcctctactatgactgttcggcccacttcctatgggctggagagcgaacccgtcagttggatggtgcccatgtggagttcctccgaggcattgccaaccctttgggtatcaaggttagcgacaaaatggacccaaaagaacttgtgaaattgattgatatcttgaatcccgaaaacaggccaggaagaataactatcattacgagaatgggacctgaaaacatgagagtgaaactcccccacctcatacgtgctgttcgtggtgctggccagatagtaacatgggtaactgacccaatgcacgggaacacgatgaaggccccttgtggcctcaagactcgctccttcgacaaaattttggctgaggtgcgcgcattctttgatgtccacgagcaagaagggagccacccaggaggggtgcatctggagatgaccgggcaaaacgtgacggagtgcatcggcgggtcacgcacggtgacgttcgatgatctgagctctcgctaccacacgcactgtgacccgaggctcaatgcctcacagtcgctggagctggtgttcatcatcgccgagcgcctcagaagaagaagggccgcctcgtgggcattggacaaccagccgggcaccattccatcatccatgggactctga

SEQ ID NO: 7 - protein, 511 aa, Brachypodium distachyon
MAAPALPVAPPVPAHAPLVLATRRRSPPSASDPPRRPRAGPLVRATSVAAAESGWAPGSWRSRPVRQIPEYPDAAALEEAERVLASFPPLVFAGEARMLEERLGDAAVGRAFLLQGGDCAESFKEFGANNIRDTFRLMLQMAVVLTFGGQMPTIKVGRMAGQFAKPRSNPVETIDGVTLPSYQGDIINNDAFDEKSRAPDPQRLIRAYSQSASTLNLLRGFAHGGYADLQRVTQWNLDFLRHSLQGERYVELAQRVQDAIGFMFAAGLPRQHPMMTTAEFWTSHECLHLPYEQALTREDSISGLYYDCSAHMLWVGERTRQLDGAHVEFLRGISNPLGVKVSDKLEPSELVELCEILNPHNKPGRLTLITRMGAENMRVKLPHMIRAVRQAGIIVTWVSDPMHGNTISAPCGLKTRSFDAIRAELRAFFDVHEQEGSYPGGVHLEMTGQNVTECIGGSNTVTFDDLSSRYRTHCDPRLNASQSLELAFAIAERLRNKRDRTWNSLISRVEA

SEQ ID NO: 8 - DNA, 1536 bp, Brachypodium distachyon
  source 1..1536: mol_type = other DNA
atggcggctcccgctctccccgtcgcgccgcccgttcccgcgcacgcgccgctcgtcctcgccacccgccggaggtcgccgccgtcggcctccgatccgccgcggcgccctagggcaggcccgctggtacgcgcgacgtcggtcgcggcggcggagagtgggtgggcgccgggcagctggaggtcgcggccggtacggcagatcccggagtacccggacgcggcggcgctggaggaggcggagcgcgtgctggcgtcgttcccgccgctggtgttcgcgggggaggcacggatgctggaggagcggctcggggatgccgccgtgggtcgcgccttcctcctgcagggcggcgactgcgccgagagcttcaaggagttcggcgccaacaacatccgcgacaccttccgcctcatgctccagatggccgtcgtcctcaccttcggtggccagatgcccaccatcaaggttgggaggatggctggccaatttgcaaagccaagatcaaacccagttgagactatagatggagtgacacttccttcctatcaaggggatatcatcaataacgatgcttttgacgagaaatcgcgcgcaccagatcctcaaaggttgatcagagcctacagccagtctgcgagcaccctgaatcttttgagaggatttgcccatggaggatatgccgatcttcagagagtcacccagtggaaccttgacttcttgaggcacagcttgcagggagaaaggtatgtggagcttgcccagagggttcaagacgccattgggttcatgtttgctgctggtctgcctcgtcagcaccccatgatgaccacagctgaattctggacatctcatgagtgtcttcacttgccatacgagcaggcactgaccagggaggactccatttctggcctctactatgactgctctgcccacatgctctgggttggagagaggactaggcagctggatggtgctcatgttgaattcctccgtggcatttccaatcctcttggtgtaaaggtgagtgataagcttgagccatcagagcttgtggaactgtgtgaaattttgaatcctcacaacaagcctggtaggctgacacttatcacaagaatgggggctgagaacatgcgtgtcaagctcccccatatgatcagagcagtgcgccaagctgggataattgtcacctgggtcagcgatcccatgcacgggaacaccatcagtgcaccgtgcgggctcaagacaagatcatttgacgcaatcagggctgagcttagggctttctttgatgttcatgagcaagaggggagctaccccggaggagttcacctggagatgacagggcagaacgttacagagtgcattggtggatcaaatacggtgaccttcgatgatctcagctcccgatatcgcacgcactgcgaccctaggctgaatgcgtcacagtcgctcgagctggccttcgccattgctgagaggctaaggaataagagagacaggacatggaatagcttgatatctagagtagaagcttaa

SEQ ID NO: 9 - protein, 540 aa, Sorghum bicolor
MALATNSAAAAAAAAAVSGGASSQPRRAAVFLPLKRRTISAIHAADPSKNNGPAVPAAAAAKSSASAVATPEKKPAAPGKWAVDSWKSKKALQLPEYPNQEELDTVLKTIETFPPVVFAGEARHLEERMAEAAMGRAFVLQGGDCAESFKEFHANNIRDTFRILLQMGAVLMFGGQVPVVKVGRMAGQFAKPRSEPFEERDGVKLPSYRGDNVNGDDFTEKSRVPDPQRMIRAYAQSVATLNLLRAFATGGYAAMQRVTQWNLDFMDHSEQGDRYRELAHRVDEALGFMTAAGLTVDHPIMTTTDFWTSHECLLLPYEQALTREDSTSGLFYDCSAHMLWVGERTRQLDGAHVEFLRGVANPLGIKVSDKMNPSDLVKLIEILNPSNKPGRITIITRMGAENMRVKLPHLIRAVRNAGLIVTWITDPMHGNTIKAPCGLKTRPFDSILAEVRAFFDVHDQEGSHPGGIHLEMTGQNVTECIGGSRTVTFDDLSDRYHTHCDPRLNASQSLELAFIIAERLRKRRMRSGLNNSLPLPPLAF

SEQ ID NO: 10 - DNA, 1623 bp, Sorghum bicolor
  source 1..1623: mol_type = other DNA
atggcgctggccaccaactccgccgccgccgccgcagcagcagcggccgtatccggtggcgcgtcatcccagccgcgccgcgcggccgtgttcctcccgctgaagaggcgcaccatctccgccatccacgccgccgacccgtcaaagaacaacggacccgccgtccccgcggcggccgctgccaagtcttccgcctcggcggtggccacgccggagaagaagccggcggctccggggaagtgggcggtcgacagctggaaatcgaagaaggcgctgcagctccccgaatacccgaaccaggaggagctggacacggtgctcaagaccatcgaaacgttcccgccggtcgtgttcgccggagaggcgcgccacctcgaggagcgcatggcagaggctgccatgggccgcgccttcgtcctccagggcggcgactgcgccgagagcttcaaggagttccacgccaacaatatccgtgacaccttccgtattctgctccagatgggcgccgtgctcatgttcggtggtcaggtgccggtcgtcaaggtggggaggatggctggccagttcgccaagccaaggtccgaaccgttcgaggagagggatggtgttaagctgccgagctacaggggtgacaacgtcaacggcgatgacttcaccgagaagagccgcgtgccagacccgcagaggatgatccgcgcctacgcgcagtcggtggcgacgctcaacctgctccgcgcgttcgccacaggagggtatgctgccatgcagcgtgtcacacaatggaacctcgacttcatggatcacagcgagcaaggtgataggtaccgtgaattggcccatagggtggatgaggctcttgggttcatgactgcagcaggacttaccgttgaccacccgataatgacgactactgacttctggacctcgcacgagtgccttctcttaccctatgagcaggctcttacccgtgaggactccaccagtggccttttctatgattgttcagcccacatgttgtgggttggtgagcgcactcgtcaacttgatggagctcatgttgaattcctccgtggtgtggccaaccctcttggcataaaggtgagtgacaaaatgaatcccagtgacttggtgaagctgattgagattctgaacccttcaaacaaacccggaaggatcaccataattacaaggatgggggcagagaacatgagagtaaagttgcctcatctcatccgtgctgtccgcaatgctggattgattgtcacatggattactgatcctatgcatggaaacaccataaaggccccttgtggcttgaagactcgtcctttcgattccattctggctgaagtgcgcgcattcttcgacgtgcatgaccaagaaggaagtcacccaggaggtatccaccttgaaatgactgggcagaacgtgaccgagtgcattggtggatcacggactgtgacctttgacgacctgagcgaccgctaccacacccactgtgacccaagactgaacgcctcccagtccctggagcttgccttcatcattgcagagaggctcaggaagaggaggatgcggtcagggctcaacaacagcctgccgctgccaccactggctttctga

SEQ ID NO: 11 - protein, 551 aa, Sorghum bicolor
MALSTNAAAAAAAISGGSASASQPARRTPSSLLPLTRRRRAVVRAVHAAEPSKNPGVVVPAAAKASSPTTVAPENDAAPAPAPARAPAKWAVDSWRTKKALQLPEYPNPAELEAVLKTIEAFPPIVFAGEARHLEERLADAAMGRAFLLQGGDCAESFKEFNSNNIRDTFRVLLQMSAVLMFGAQMPVVKVGRMAGQFAKPRSDPFEVRDGVKLPSYRGDNINGEAFDEKSRVPDPQRMIRAYAQSAATLNLLRAFATGGYAAMQRVTQWNLDFTEHSEQGDRYRELAHRVDEALGFMSAAGLTPDHPLTTTTEFWTSHECLLLPYEQALTRQDSTSGLFYDCSAHMLWVGERTRQLDGAHVEFLRGIANPLGIKVSDKMNPSDLVKLIDILNPTNKPGRITVITRMGAENMRVKLPHLIRAVRQAGQIVTWITDPMHGNTIKAPCGLKTRPFDNILAEVRAFFDVHEQEGSHPGGVHLEMTGQNVTECIGGSRTVTFDDLSDRYHTHCDPRLNASQSLELAFIIAERLRKRRIRSSSGLNNILPLPPFGF

SEQ ID NO: 12 - DNA, 1656 bp, Sorghum bicolor
  source 1..1656: mol_type = other DNA
atggcgctctccaccaacgccgccgccgccgccgccgccatctccggcgggtccgcgtccgcgtcgcagccggcccgtcgcacgccctcgtctctcctcccgctgacgcgccgccgccgcgccgtcgtccgcgccgtgcacgccgcggagccgtcgaagaaccccggcgtcgtcgtcccggccgccgccaaggcctcgtcgccgacgacggtcgcgcctgagaacgacgcggcccctgctccggccccggcccgtgcgccggcgaagtgggcggtggacagctggaggacgaagaaagcgctacagctgccggagtatccgaacccggcagagctagaggcggtgctcaagaccatcgaggcgttcccacccatcgtcttcgctggggaggcgcgccacctggaagagcgcctcgccgacgccgccatgggacgcgccttcctcctccagggcggcgactgcgctgagagcttcaaggagttcaacagcaacaacatccgcgacaccttccgcgtcctcctgcagatgtccgccgttctcatgttcggcgcccagatgcccgtcgtcaaggttggtaggatggccggccaattcgcgaagccaaggtcggatccgttcgaggttagagatggcgtcaagctgcccagctaccggggcgataacattaatggcgaggcattcgacgagaagagccgtgtccccgacccacagaggatgatccgcgcgtatgcgcagtctgccgcaacgctgaacttgctccgcgccttcgccaccggaggctacgctgccatgcagcgcgtcacgcagtggaacctcgatttcaccgaacacagcgagcagggcgacaggtaccgtgaattggcacatcgggttgatgaagcccttggcttcatgtctgcagctggcctaacaccggaccaccctttgacgacgaccactgagttctggacctcacatgagtgcctcctcctaccttacgagcaagctttaacccgtcaagactccacctctggtcttttctacgattgctctgcccacatgctctgggttggtgagcgcactcgccagcttgatggcgcccatgttgagttcctcaggggtattgccaaccctcttggcatcaaggtaagtgacaaaatgaaccccagcgacttggtgaagctgattgatatactgaacccaacaaacaagcccgggaggatcaccgttattacaaggatgggggcagagaacatgagggtgaagttacctcaccttatccgtgcggtccgccaggctggacaaattgtcacctggatcactgacccgatgcacggcaacaccatcaaggctccttgtggtctaaagactcgtccatttgacaatattctggctgaggtacgggccttctttgatgtgcacgagcaagaggggagccaccctggaggtgtccaccttgagatgactgggcaaaacgtaactgaatgcattggcgggtcacgcaccgtgaccttcgatgacctgagcgaccgctaccacacccactgtgaccccaggctgaacgcgtcccagtctctggagctcgcgttcattatcgccgagaggctgaggaagaggaggatccggtcatcgtccgggctcaacaacatcttgcccttgccgccttttggtttctga

SEQ ID NO: 13 - protein, 508 aa, Sorghum bicolor
MPLAPSTPALPPNPALPSPCRGQGRGRPGRGALLRARAVRAAPRPPSQWSVGSWRGRPALQQPEYPDKADLNEVLRTVEAFPPIVFAGEARTLEERLAEAAVGRAFLLQGGDCAESFKEFNANNIRDTFRVLLQMSVVLMFGGQMPVVKVGRMAGQFAKPRSDGFEERDGVKLPSYRGDNINGDAFDEKSRLPDPHRMISAYSQSAATLNLLRAFATGGYAAMQRVTQWNLDFTEHSEQGDRYMELAHRVDEALGFMSAAGLTLDHPIMTTTEFWTSHECLLLPYEQALTREDSTSGLYYDCSAHFLWVGERTRQLDGAHVEFLRGIANPLGIKVSDKMDPAELVRLIDILNPENRAGRITIIARMGPENMRVKLPHLIRAVRGAGQIVTWVTDPMHGNTMKAPCGLKTRSFDRILAEVRAFFDVHEQEGSHPGGVHLEMTGQNVTECIGGSRTVTFDDLGSRYHTHCDPRLNASQSLEMAFIIAERLRKRRIASSPLYTNQLGSIRL

SEQ ID NO: 14 - DNA, 1527 bp, Sorghum bicolor
  source 1..1527: mol_type = other DNA
atgccactcgcgccatcaacccccgcgctgccgcctaacccggccctcccctcgccgtgccgggggcaggggcggggccggcccggccgcggggcgctcctccgcgcccgcgccgtccgcgcggcgccgcggcccccgagccagtggtccgtcggcagctggcggggccgcccggcgctgcagcagcccgaatacccggacaaggcggacctgaacgaggtgctgcggacggtggaggcgttcccgcccatcgtgtttgccggcgaggcgcgcaccctcgaggagcgcctcgcggaggccgccgtcggcagagctttcctcctccagggcggtgactgcgctgagagcttcaaggagttcaacgccaacaatattagggacacctttcgcgtcctcctgcaaatgtctgttgtgctcatgttcggaggccagatgcctgtcgtcaaggtgggaagaatggcaggtcagtttgcaaagccaagatcagatggttttgaggagcgggatggagtgaagttgccaagctatagaggggacaatatcaatggggatgcatttgatgagaagtcaagattgccagatccacaccgcatgataagtgcctactcacagtctgcagcaacgctgaatctgctgcgggcgttcgcaactggaggttatgctgcaatgcagagggtaacacaatggaaccttgatttcacagagcatagtgaacaaggggataggtacatggaattggctcaccgagttgacgaagctttggggttcatgtcagctgctggactcactttagatcaccctataatgacgacaacagaattctggacgtcacatgagtgccttcttctgccttatgagcaagcgctcacccgtgaagactccaccagtggcctctattatgattgctctgctcacttcctatgggttggagagcgcactcgccagcttgatggtgctcatgtggagttccttcgaggcatcgccaaccctcttggtatcaaggttagtgacaagatggacccagcagaacttgtgcggttgattgatatattgaatcccgaaaacagggctgggagaataaccatcatcgcaagaatgggacctgaaaacatgagggtgaaacttccacacctgatacgcgccgtccgtggggccggccagatagtaacatgggtcactgacccaatgcatgggaacaccatgaaggccccttgcggactcaaaacccgctcatttgacagaattttggccgaggtgcgtgcgttctttgatgtccacgaacaagaagggagccaccctggaggagtgcatctagagatgactgggcaaaatgttacagagtgcattggagggtcacgtaccgtgacatttgatgatctgggctcacgctaccacacgcactgcgacccaaggctcaacgcctcacagtctctggagatggcattcattatcgcggagcgccttaggaaaaggaggattgcctcgtcgcctttgtacacgaaccagctgggttccattcgtctgtga

SEQ ID NO: 15 - protein, 499 aa, Sorghum bicolor
MAPLATAPPPTPHAPRSLVPRVRPRRGLTAVRAASQGRTTDGWTPGSWRARPARQIPEYPDTAALEATERTLAEFPPLVFTGEVRKLEERLGEAAMGRAFLLQGGDCAESFREFNAQKIRDTFRLILQMAVVLTFGGQMPTIKVGRMGGQFAKPRSNPTETVDGVTLPSYRGDIINDQAFDEKSRVPDPERLIRAYTQSASTLNLLRAFAHGGFADLQRVTQWNLDFLRHSTQGDRYLELSQRVHEAIGFMVAAGLTPQHPIMTTAELWTSHESLHLPYEQALTREDSISGRYYDCSAHMLWVGERTRQLDGAHVEFLRGISNPLGIKVSDKLDPSELVKLCETLNPHNKPGRLTIITRMGAENMRVKLPQMIRAVRQAGMIVTWVSDPMHGNTISAPCGLKTRSFDSIMAELRAFFDVHGLEGSYPGGVHLEMTGQNVTECIGGSKAVTFDDLSARYHTHCDPRLNASQSLELAFAIADRLRNKRDKTWHNLTSRVVA

SEQ ID NO: 16 - DNA, 1500 bp, Sorghum bicolor
  source 1..1500: mol_type = other DNA
atggcgcctctcgccaccgcgccgcctcctactccccatgccccgcgctctctcgttccacgggtaaggccgcggcgagggctgacggcagtgcgcgcggcctcacagggaagaacaacggatgggtggacgccgggaagctggcgtgcgcgccctgcgcggcagatcccggagtaccccgacacggcggcgctggaggctacagagcgaactctggcggagtttccaccgctagtgttcacgggtgaggtgcggaagctggaggagcggctcggggaggcggccatgggtcgggcattcctcctgcagggcggcgactgcgccgagagctttagggagttcaatgcacagaaaatccgggacaccttccgactcatactgcagatggccgtcgttctcaccttcggcggacagatgcccaccatcaaggtgggaaggatgggtggtcaatttgcgaagccaagatcgaacccaactgagactgttgatggggtaacccttccctcctatagaggggatatcatcaacgatcaggcttttgatgagaaatctcgtgtaccagatcctgaaaggttgattagagcctatacccagtctgcgagcaccctgaatcttctcagagcatttgcccatggaggttttgcggatcttcagagagtcacccagtggaatctcgactttttgaggcacagcacacaaggagacaggtatctggagctttctcagagggttcatgaagccattgggtttatggttgccgctgggctgacccctcaacaccccatcatgaccacagctgagttatggacatcccatgagtcccttcacttaccatacgagcaagcactgactagggaggattccattagtggccggtactatgactgttccgcacacatgctttgggtcggtgagaggactcggcagctggatggtgctcatgttgaattccttcgtggtatttcaaatcctctgggtataaaggtaagtgataagcttgacccatcagagcttgtgaagttgtgtgagactctgaatcctcacaacaagcctgggcgattgacgattatcacaagaatgggggctgagaacatgcgtgttaagcttccacaaatgatcagagcagtgcgccaagctgggatgattgttacttgggttagtgatcctatgcacggaaacactatcagtgcaccatgcggactgaagacaagatcatttgattcaatcatggctgaactcagggctttcttcgatgttcatgggctagagggaagctaccctggaggagttcacctggagatgacggggcagaatgttacagagtgtatcgggggatctaaggcggtgacatttgatgatctcagtgcccgctaccacacacactgtgaccccagactgaacgcgtcgcagtccctcgagctggctttcgccattgctgacaggttaaggaacaagagagacaagacatggcataatttgacatccagagttgttgcttaa

SEQ ID NO: 17 - protein, 537 aa, Oryza sativa
MALATNSAAVSGGAAAAASSAPQPRLAATFLPMRRRTVSAVHAADPAKSNGPVQAAAKASSPSTVAAPEKKPVGLGKWTVDSWKAKKALQLPEYPSQEELDSVLKTIETFPPVVFAGEARHLEERLADAAMGRAFVLQGGDCAESFKEFNANNIRDTFRILLQMGAVLMFGGQMPVVKVGRMAGQFAKPRSDSFEERDGVKLPSYRGDNINGDTFDEKSRVPDPQRMIRAYAQSVATLNLLRAFATGGYAAMQRVTQWNLDFMDHSEQGDRYRELAHRVDEALGFMTAAGLTVDHPIMTTTDFWTSHECLLLPYEQSLTREDSTSGLFYDCSAHMLWVGERTRQLDGAHVEFLRGVANPLGIKVSDKMNPRDLVKLIEILNPSNKPGRITIITRMGAENMRVKLPHLIRAVRNSGQIVTWITDPMHGNTIKAPCGLKTRPFDSILAEVRAFFDVHDQEGSHPGGIHLEMTGQNVTECIGGSRTVTFDDLSDRYHTHCDPRLNASQSLELAFIIAERLRRRRMRSGVNSNLPLPPLAF

SEQ ID NO: 18 - DNA, 1614 bp, Oryza sativa
  source 1..1614: mol_type = other DNA
atggcgctcgccaccaactccgccgctgtttccggtggcgctgcggccgcggcgtcgtcggcgccccagccgcggctcgccgccacgttcctcccgatgaggaggcgaaccgtctcggcggtccacgcggccgacccggcgaagagcaacgggcccgtgcaggccgcggcgaaggcctcgtccccgtcgacggtggcggcgccggagaagaagccggtggggttggggaagtggacggtggatagctggaaggcgaagaaggcgctgcagctgcccgagtacccgagccaggaggagctcgactccgtgctcaagacgatcgagacgttcccgcccgtggtgttcgccggggaggcgcgccacctcgaggagcgcctcgccgacgccgccatgggccgcgccttcgtcctccagggcggcgactgcgccgagagcttcaaggagttcaacgccaacaacatccgtgacaccttccgcatcctgctccagatgggcgccgtcctcatgttcggcggccagatgcccgtcgtcaaggtcgggaggatggctggccagttcgccaagccgaggtctgattcgttcgaggagagggacggggttaagctgccgagttacaggggagacaacatcaatggcgacaccttcgacgagaagagccgcgtgccggacccgcagcggatgatccgcgcgtacgcgcagtcggtggcaacgctcaacctgctccgcgccttcgccacgggagggtacgccgccatgcagcgcgtcacgcagtggaacctcgatttcatggatcacagcgaacaaggagacaggtaccgtgaattggcacaccgggtggatgaggcacttggcttcatgactgcagcagggctaacagtcgaccatccgataatgacaacaactgacttctggacatcccatgagtgcctcctcttaccatatgagcagtctcttacacgtgaggattccaccagtggcctcttctatgactgctcagcccacatgctatgggttggtgagcgcactcgccagctcgatggagctcatgttgaatttctccgtggtgttgccaaccctcttggcataaaggtgagtgacaaaatgaacccccgtgacttggtgaagctcattgagattttgaacccatcaaacaagcctggaaggataaccataattacaagaatgggggcagagaacatgagggtgaaattgcctcatcttatccgtgctgtccgcaactctggacagattgtcacatggattactgatcccatgcatggaaacaccatcaaggctccatgtggtctgaagactcgtccattcgattccattctggctgaagtacgtgcgttcttcgatgtccatgatcaagaaggtagccacccaggaggtatccacctggaaatgactgggcagaacgtgactgaatgcatcggtggatcaaggaccgtcaccttcgatgacctgagcgaccgctaccacacccactgtgacccaaggctgaacgcgtcgcaatccctggagctcgccttcatcatcgccgagagactgaggcggaggaggatgcggtccggggtcaacagcaacctgccattgcccccattggctttctaa

SEQ ID NO: 19 - protein, 555 aa, Oryza sativa
MSLATSSSMAGGAAVVPRSATATTASAFVTMKRRATAVRAVHAAEPSKNPPVGVPSAAKTSSPSVAAPEKAPVAAAPAPVAPAPAATKQVAPARWAVDSWRTKKALQLPEYPNAAELEAVLKTIEAFPPIVFAGEARHLEERLADAAMGRAFLLQGGDCAESFKEFNGNNIRDTFRVLLQMSAVLTFGGQMPVIKVGRMAGQFAKPRSEAFEERDGVKLPSYRGDNINGDAFNEKSRIPDPQRMVRAYAQSAATLNLLRAFATGGYAAMQRVTQWNLDFTQHSEQGDRYRELAHRVDEALGFMSAAGLTVDHPLMTSTDFWTSHECLLLPYEQSLTRQDSTTGHFYDCSAHMLWVGERTRQLDGAHVEFLRGVANPLGIKVSDKMNPTELVKLIEILNPSNKPGRITIITRMGAENMRVKLPHLIRAVRHAGQIVTWITDPMHGNTIKAPCGLKTRPFDSILAEVRAFFDVHDQEGSHPGGVHLEMTGQNVTECIGGSRTVTFDDLGDRYHTHCDPRLNASQSLELSFIIAERLRRKRIRSSKLNNMLPLPPFGV

SEQ ID NO: 20 - DNA, 1668 bp, Oryza sativa
  source 1..1668: mol_type = other DNA
atgtcgctcgccaccagctcctcgatggccggtggggcggcggtggttccacgcagcgcgacggcgacgacggcctcggcgttcgtcacgatgaagcggcgcgccacggccgtgcgcgccgtacacgcggcggagccgtcgaagaacccgcccgtcggcgtcccgtccgcggcgaagacgtcgtcgccgtcggtggcggcgccggagaaggcccccgtggcggccgcgcccgcgcctgtggctccggctccggcggccacgaagcaggtggcgccggcgaggtgggcggtggacagctggaggacgaagaaggcgctgcagctgccggagtaccccaacgcggcggagctggaggcggtgctcaagacgatcgaggcgttcccgccgatcgtgttcgccggggaggcgaggcacctggaggagcgcctcgccgacgccgccatgggccgcgccttcctcctccagggcggcgactgcgccgagagcttcaaggagttcaacggcaacaacatccgcgacaccttccgcgtcctcctccagatgtccgccgtcctcaccttcggcggccaaatgcccgtcatcaaggttgggagaatggcggggcaattcgcgaagccgaggtcggaggcgttcgaggagagggacggggtgaagctgccgagctacaggggcgacaacatcaatggcgacgcgttcaacgagaagagccgcatccctgacccgcagaggatggtccgggcctatgcgcagtccgccgccacgctcaacctcctccgcgctttcgccaccggaggatacgccgccatgcagcgcgtgacgcagtggaacctcgatttcacccagcacagcgagcagggcgacaggtaccgtgaattggcacacagggttgatgaagcccttggctttatgtctgctgctgggctaacagtggaccacccgttgatgacaagtactgatttctggacctcacatgagtgccttctcctaccctatgaacaatctctgacccgtcaggactccacaactggtcatttctacgactgctctgcccacatgctatgggtgggcgaacgcactcgccagctcgatggtgctcatgttgagttcctcaggggtgtggccaaccctcttggcatcaaggtgagtgacaaaatgaacccaaccgagttggtgaagctgattgagattttgaacccatcaaacaagcctgggagaattaccatcatcacaaggatgggggcagagaacatgagggtcaagttacctcatcttatccgtgcggtccgccacgctggacaaattgtcacatggattactgatccaatgcacgggaacaccatcaaggctccttgtggtctgaagacacgcccctttgactccattctggccgaggtacgagcattcttcgatgtgcatgatcaagaaggaagccacccaggaggtgtccacctcgagatgactgggcagaacgtaaccgagtgcatcggcggatcccgaaccgtcaccttcgatgacctgggtgaccgctaccacacccactgcgacccgaggctgaacgcgtcccagtcgctggagctctccttcatcatcgccgagaggctgaggaggaagaggatccggtcgtcgaagctgaacaacatgttgccgttgccacccttcggtgtctga

SEQ ID NO: 21 - protein, 505 aa, Oryza sativa
MPLAPCPSPPLPSSPWPARAPRRGGLLRARAVRAAPRPPSKWSLGSWRSLTALQQPEYPDKAELDEVLRTVEAFPPIVFAGEARKLEERLAEAAVGRAFLLQGGDCAESFKEFNANNIRDTFRVLLQMSVVLMFGGQMPIIKVGRMAGQFAKPRSDGFEERDGVKLPSYRGDNINGDSFDEKSRLPDPHRMIRAYSQSAATLNLLRAFATGGYAAMQRVTQWNLDFTEHSEQGDRYMELAHRVDEALGFMAAAGLTMDHPIMTTTEFWTSHECLLLPYEQALTREDSTSGLYYDCSAHFLWVGERTRQLDCAHVEFLRGIANPLGIKVSDKMDPKELVKLIDILNPQNKPGRITIITRMGPENMRVKLPHLIRAVRGAGQIVTWVTDPMHGNTMKAPCGLKTRSFDRILAEVRAFFDVHEQEGSHPGGVHLEMTGQNVTECIGGSRTVTFDDLGSRYHTHCDPRLNASQSLELAFIIAERLRKRRIASWQLNKNSHLGNIPSLGL

SEQ ID NO: 22 - DNA, 1518 bp, Oryza sativa
  source 1..1518: mol_type = other DNA
atgcccctcgcgccatgcccctcgccgcccctcccctcctccccgtggccggcgcgcgccccgcgccggggcggcctcctccgcgcccgcgcggtgcgggcggcgccccggccgccgagcaagtggtcgctgggtagctggcgcagcctgacggcgctgcagcagccggagtaccccgacaaggcggagctggatgaggtgctccggacggtggaggcgttcccgccgattgtcttcgccggcgaggcgcgcaagctggaggagcggctcgcggaggccgccgtcgggcgcgcgttcctcctccagggcggcgactgcgccgagagcttcaaggagtttaacgcgaacaacatccgggacaccttccgtgtgctcctccagatgtccgtggtgctcatgtttggaggccagatgcctatcatcaaggtaggaagaatggcaggtcagtttgcaaagccaaggtcagatggctttgaggagagggatggagtgaagttgccaagctacagaggggataacattaatggggattcattcgatgagaaatcaagattgccagatccacaccgcatgatcagggcatactcacagtctgcagcaacactgaatttgctgcgggcttttgctactggaggttatgctgccatgcagagggtaacacaatggaaccttgacttcacagagcatagtgaacagggtgacaggtacatggagctggctcaccgagttgatgaggctttggggttcatggcagctgctggtctcactatggaccatcctattatgacaacaacagaattctggacatcacatgagtgccttcttcttccctatgagcaagcacttactcgcgaggattccacatctggcctctattacgactgttctgctcacttcctttgggttggagagcgtacacgtcagcttgattgtgcccatgtggagtttctccgaggaattgcgaaccctctgggtatcaaggtcagtgacaagatggacccaaaagaacttgtgaagttgattgatatcttgaatccccagaacaaaccagggagaattactatcattacaagaatgggacctgaaaacatgagagtgaaactccctcacctaatacgtgctgtccgtggtgctggccagatagtaacatgggttactgatccgatgcatggtaacacaatgaaggctccttgtggcctcaagactcgctcctttgatagaatcttggctgaggtgcgcgcattctttgatgtgcacgaacaagaaggcagccacccaggaggggtgcatctggagatgactggacaaaatgtgacagaatgcatcggcgggtcacgcacggtgacattcgacgatctgggctcacgataccacacacactgcgacccgaggctcaacgcatcgcagtctctggagttggcgttcatcatcgccgagcggctcagaaagaggaggatcgcctcatggcagttgaacaagaacagtcatctgggcaacatcccatctttggggctctga

SEQ ID NO: 23 - protein, 508 aa, Oryza sativa
MTCHSAMAALTVGHAAIVHATTRLEDARSTGRRRRRRGMITVRAAAAATSGWEPGSWRARPARQIPEYPDAAALEGAERELASFPPLVFAGEARKLEERLGDAAMGRAFLLQGGDCAESFKEFAANNIRDTFRLMLQMAVVLTFGGQMPTIKVGRMAGQFAKPRSNPTETIDGVTLPSYRGDIINSDGFDEKSRAPDPERLIRAYSQSASTLNLLRGFAHGGYADLQRVTQWNLDFLRDSTQGDRYMELSERVHDAIGFMVAAGLTPQHPIMTTAEFWTSHECLHLPYEQALTRVDSISGLYYDCSAHMLWVGERTRQLDGAHVEFLRGISNPLGVKVSDKLEPSELVKLCEILNPHNKPGRLTIITRMGAENTRVKLPHMIRAVRQAGLIVTWVSDPMHGNTISAPCGLKTRSFDAIRCELRAFFDVHEQEGSYPGGIHLEMTGQNVTECIGGSKTVTLDDLSSRYRTHCDPRLNASQSLELAFAIADRLRKKRDRAWNRLVYRAVA

SEQ ID NO: 24 - DNA, 1527 bp, Oryza sativa
  source 1..1527: mol_type = other DNA
atgacgtgtcactctgccatggcggctctcaccgttgggcacgcggcgatcgtccatgccaccacgcgtctcgaggacgcccggtccaccggccgtcggcggcggcggcgggggatgatcacggtgcgcgcggcggcggcggcgacgagcgggtgggagccgggcagctggagggcgcggccggcgaggcaaatcccggagtacccggacgcggcggcgctggagggcgcggagcgcgagctggcgtcgttcccgccgctggtgttcgccggggaggcgcggaagctggaggagcggctcggggacgccgccatggggcgggccttcctcctgcagggcggcgactgcgccgagagcttcaaagagttcgccgccaacaacatccgcgacacctttcgcctcatgctccagatggccgtcgtcctcacctttggcggccagatgcccaccatcaaggttggaaggatggctggccaatttgcaaagccaagatcaaacccaactgagactatagatggagtgacacttccttcctatcgaggtgatattattaacagcgatggttttgatgagaagtcgcgtgcaccagatcctgaaaggttaattagagcctacagccagtccgcgagcaccctgaatcttctgagaggatttgctcatggagggtatgcagatctgcagagagtcacccagtggaatcttgacttcttgagggacagcacgcaaggggacaggtatatggagctgtccgagagggttcacgatgccatcggatttatggttgctgctggtctgactcctcagcatcccatcatgacgacggctgaattctggacatctcatgagtgccttcatttgccatatgagcaagcattgactagggtggactccatttctgggctttactacgattgctctgctcatatgctttgggttggggagaggactcgacaactggatggtgctcatgttgaattccttcgtggtatttccaaccctctaggtgtaaaggtgagtgacaagctcgaaccttcagagcttgttaaattgtgtgagattttgaatcctcacaacaagcccggaagactgacaatcatcacaagaatgggtgctgagaacacgcgcgttaagctcccacatatgatcagagcagtccgccaagctgggttgattgtcacttgggtcagtgatcccatgcatgggaacaccatcagcgcaccatgtggtctcaagacaagatcattcgacgcgatcaggtgcgagctgagggctttcttcgatgtccatgagcaagagggaagctaccctggagggattcacctggagatgacagggcagaacgttacagagtgcattggtggatccaagacggtgacccttgatgatctcagctctcgctaccgcacgcactgcgaccccaggctgaatgcatcgcagtcgcttgaactggctttcgccattgctgacaggctaaggaagaaacgagacagggcttggaataggttggtgtacagggcggtagcttaa

SEQ ID NO: 25 - protein, 539 aa, Setaria viridis
MALATNSAATAAAAAAVSGGAASQPRRAAAFVPLKRRTISAIHAADPSKNNGSAVPAASSSKASSFAVATPEKKPAAQGKWTVDSWKSKKALQLPEYPSQEELDSVLKTIETFPPVVFAGEARHLEERLAEAAMGRAFVLQGGDCAESFKEFHANNIRDTFRILLQMGAVLMFGGQVPVVKVGRMAGQFAKPRSEAFEERDGVKLPSYRGDNVNGDDFTEKSRVPDPQRMIRAYAQSVATLNLLRAFATGGYAAMQRVTQWNLDFMDHSEQGDRYRELAHRVDEALGFMTAAGLTVDHPIMTTTDFWTSHECLLLPYEQALTREDSTSGLFYDCSAHMLWVGERTRQLDGAHVEFLRGVANPLGIKVSDKMNPSDLVKLIEILNPSNKPGRITIITRMGAENMRVKLPHLIRAVRNAGLIVTWITDPMHGNTIKAPCGLKTRPFDSILAEVRAFFDVHDQEGSHPGGIHLEMTGQNVTECIGGSRTVTFDDLSDRYHTHCDPRLNASQSLELAFIIAERLRKRRMRSGLNNSLPLPLAF

SEQ ID NO: 26 - DNA, 1620 bp, Setaria viridis
  source 1..1620: mol_type = other DNA
atggcgctcgccaccaactccgcggcaaccgccgccgccgcggcggccgtatccggcggcgcggcatcccagccgcgccgcgcggccgcgttcgtcccgctgaagaggcgcaccatctccgccatccacgccgccgacccgtcgaagaacaacgggtccgccgtccccgccgcctcctcctccaaggcctcgtcctttgcggtggcgacgccggagaagaagccggcggcgcaagggaagtggacggtagacagctggaagtcgaagaaggcgctgcagctccccgagtacccgagccaggaggagctggactccgtgctcaagacgatcgagacgttcccgccggtggtgttcgccggggaggcgcgccacctcgaggagcgcctcgccgaggccgccatgggccgcgccttcgtcctccagggtggagattgcgctgagagcttcaaggagttccacgccaacaacatccgtgacaccttccgcatcctgctccagatgggcgctgtcctcatgttcggcggtcaggtcccggtcgtcaaggtggggaggatggctggccagttcgccaagccaaggtccgaggcattcgaggagagggacggcgttaagctgccgagctacaggggcgacaacgtgaacggagacgatttcaccgagaagagccgcgtgccggacccgcagaggatgatccgcgcctacgcgcagtcggtggccacgctcaaccttctccgcgcgttcgcgactggaggctacgctgctatgcagcgcgtcacacagtggaatctcgatttcatggatcacagcgagcaaggtgataggtaccgtgaattggcccatagggtggatgaggctcttggattcatgactgcagcggggcttacagttgaccacccgataatgacgactactgacttctggacctcgcacgagtgccttctcttaccatacgagcaggctcttacccgtgaggactccaccagtggccttttctatgattgttcggcccacatgctgtgggttggggagcgcactcgccaacttgatggggctcatgttgaattcctcagaggtgttgccaaccctcttggcataaaggtgagcgacaaaatgaaccccagtgacttggtgaagctgattgagattttgaacccttcaaacaaacctggaagaatcaccataattacaaggatgggggcagagaacatgagagtgaagttgcctcatcttatccgtgctgtccgcaatgctggactgattgtcacatggattactgatcctatgcatggaaacacaatcaaggccccttgtggtttgaagactcgtccattcgactccattcttgctgaagtacgtgcattcttcgatgtgcatgaccaagaagggagccacccaggaggtatccacctggagatgactgggcagaacgtgactgagtgcattggtggatcacggaccgtgaccttcgatgacctgagcgaccgctaccacacccactgtgacccaaggctgaatgcctcccagtcccttgagcttgccttcatcattgccgagaggctcaggaagaggaggatgcggtcagggctcaacaacagcctgccgctgccactggctttctaa

SEQ ID NO: 27 - protein, 548 aa, Setaria viridis
MALATNAAAAAAAAAAISGAAASQPSRAPSFLPMRRRCAVRAVHAAEPSKSHGVPAAAKTSAPTVAPEKEAAPVAAPAPAPKAPAKWAVDSWRSKKALQLPEYPNAAELEAVLKTIEAFPPIVFAGEARHLEERLADAAMGRAFLLQGGDCAESFKEFNSNNIRDTFRVLLQMSAVLMFGAQMPVVKVGRMAGQFAKPRSDPFEVRDGVKLPSYRGDNINGEAFDEKSRVPDPQRMIRAYAQSAATLNLIRAFATGGYAAMQRVTQWNLDFTEHSEQGDRYRELAHRVDEALGFMSAAGLTADHTLMKTTEFWTSHECLLLPYEQALTRQDSTSGLFYDCSAHMLWVGERTRQLDGAHVEFLRGIANPLGIKVSDKMNPSDLVKLIEILNPSNKPGRITIITRMGAENMRVKLPHLIRAVRQAGLIVTWITDPMHGNTIKAPCGLKTRPFDSILAEVRAFFDVHEQEGSHPGGVHLEMTGQNVTECIGGSRTVTFDDLSDRYHTHCDPRLNASQSLELSFIIAERLRKRRIRSSSGLNNILPLPPFGF

SEQ ID NO: 28 - DNA, 1647 bp, Setaria viridis
  source 1..1647: mol_type = other DNA
atggcgctcgccaccaacgccgccgccgccgccgccgcggcagcagccatctccggcgcggccgcgtcgcagcccagccgcgctccgtcgttcctcccgatgaggcgccgctgcgccgtccgcgccgtgcacgccgcggagccgtcgaagagccacggcgtcccggcggcggcgaagacctcggcgccgacggtcgcgcccgagaaggaggcggcccctgtcgcggccccggccccggccccgaaggcgcctgcgaagtgggcggtggacagctggaggtcgaagaaggccctgcagctgccggagtacccgaacgcggcggagctggaggccgtgctcaagacgatcgaggccttcccgccgatcgtgttcgccggggaggcgcgccacctcgaggagcgcctcgccgacgccgccatgggccgcgccttcctcctccagggcggcgactgcgccgagagcttcaaggagttcaacagcaacaacatccgcgacaccttccgcgtcctcctccagatgtccgccgtcctcatgttcggcgcccaaatgcccgtcgtcaaggttggtaggatggccgggcaattcgccaagccgaggtcggacccgtttgaggtcagggacggggtgaagctgcccagctaccggggcgacaacatcaacggcgaggcgttcgacgagaagagccgcgtccccgacccgcagaggatgatcagggcgtacgcccaatccgccgccacgctcaacctgatccgtgcgttcgccaccggagggtacgccgccatgcagcgcgtcacgcagtggaacctcgatttcaccgaacacagcgagcagggcgacaggtaccgtgaattggcacatcgggttgatgaagcccttggcttcatgtctgcagctgggctaacagcggaccacacgttgatgaagactactgaattctggacctcacatgaatgcctcctcctaccctacgaacaagctctaacccgtcaggattccacctctggtcttttctatgattgctctgcccacatgctctgggttggcgagcgcactcgccagcttgatggtgctcatgttgagttcctaaggggcattgccaacccccttggcatcaaggtgagtgacaaaatgaaccccagcgatttggtgaagctgattgagatattgaatccatcaaacaagcctgggagaattaccatcattacaaggatgggggcggagaacatgagggtcaagttacctcatcttattcgtgctgtccgccaagcaggactaattgtcacatggatcactgatcccatgcacggtaacaccatcaaggctccttgcggtctaaagactcgccccttcgactccattttggctgaggtacgggccttttttgatgtgcacgagcaagagggaagccacccaggaggcgtccaccttgagatgactgggcaaaacgtaactgaatgcatcggtggatcacgcaccgtgacttttgatgacctgagcgaccgctaccacactcactgcgacccaaggttgaacgcgtcccagtcgctggagctctcatttatcattgccgagaggctgaggaagaggaggatccggtcatcgtctggtctcaacaacatcttgcccttgccgccttttggtttctga

SEQ ID NO: 29 - protein, 506 aa, Setaria viridis
MPLAPSTPALLPNPALPSPGRPRSRGALLRARAVRAAPRPPSRWSVGSWRERPAQQQPEYPDKAELDEVLRTVEAFPPIVFAGEARTLEERLAEAAVGRAFLLQGGDCAESFKEFNANNIRDTFRVLLQMSVVLMFGGQMPIVKVGRMAGQFAKPRSDGIEERDGVKLPSYRGDNINGDTFDEKSRLPDPHRLIRAYSQSAATLNLLRAFATGGYAAMQRVTQWNLDFTQNCEQGDRYMELAHRVDEALGFMSAAGLTLDHPIMTTTEFWTSHECLLLPYEQALTREDSTTGLYYDCSAHFLWVGERTRQLDGAHVEFLRGIANPLGIKVSDKMDPAELVRLIDILNPENRAGRITIITRMGPENMRVKLPHLIRAVRGAGQIVTWVTDPMHGNTMKAPCGLKTRSFDRILAEVRAFFDVHEQEGSYPGGVHLEMTGQNVTECIGGSRTVTFDDLTSRYHTHCDPRLNASQSLEMAFIIAERLRKRRIASWNLNGNQLGSIPSLGL

SEQ ID NO: 30 - DNA, 1521 bp, Setaria viridis
  source 1..1521: mol_type = other DNA
atgccactcgcgccatcaacccccgcgctgctgcccaacccggccctcccctcgccggggcggccgcgctcccgcggggcgctcctccgcgcccgcgccgtgcgggcggcgcctcggcccccgagccggtggtcggtggggagctggcgggaacgcccggcgcaacagcagccggagtacccggacaaggcggagctggatgaggtgctgcggacggtggaggcgttcccgcccatcgtcttcgccggggaggcgcgcaccctcgaggagcggctcgcggaggcggccgtgggccgggcgttcctcctccagggcggcgactgcgccgagagcttcaaggagttcaacgccaacaacatcagggataccttccgggtccttctgcaaatgtccgtcgtgctcatgttcggaggacagatgcctatcgtcaaggtgggaagaatggcaggccagtttgcaaagccaagatcagatggtattgaggaacgggatggagtgaagctgccaagctatagaggggacaatataaatggggacacattcgatgagaagtcaagattgccagatccacaccgcctgataagggcgtactcacaatctgcagctacactgaatttgttgcgggcattcgctactggaggttatgctgcaatgcagagggtaacacagtggaaccttgacttcacccagaattgtgaacagggcgataggtacatggagttggcccaccgagttgacgaggctttgggattcatgtcagctgctggactcactttagatcaccctataatgacaacaacagaattctggacatcgcatgagtgccttcttcttccttacgagcaggcgcttactcgtgaagactccaccacaggcctctattatgactgctctgcccacttcctatgggttggagagcgtactcgtcaacttgatggtgcccatgtggagtttcttcggggcattgccaaccctcttggtatcaaggtcagtgacaagatggacccagcagaacttgtgcggttgattgatatcttgaatcctgaaaacagggcagggagaataaccatcatcacaagaatgggacctgaaaacatgagggtgaaacttcctcacctgatacgtgctgtccgtggggctggccagatagtaacatgggtcactgacccaatgcatgggaacaccatgaaggccccttgtggactcaaaacccgctccttcgacagaattttggctgaggtgcgtgcattctttgatgttcacgaacaagaagggagctatccaggaggcgtgcatctggagatgaccggacagaatgtgacagaatgcattggtggatcacggacagtgacatttgatgatctgacctcacgctaccacacacactgcgacccaaggctcaatgcctcgcaatcgctggagatggcattcatcatcgctgagcgcttgaggaaaaggaggattgcctcatggaatttgaacgggaaccagctgggttccattccatcattggggctgtaa

SEQ ID NO: 31 - protein, 489 aa, Setaria viridis
MYCMRSVSRAAPASFPLAVRSPRFDLPSLPTAPPAPPSAPRLLVPQTRPRRGPTMYPDPAALEATERALETFPPLVFAGEARKLEERLGEAFLLQGGDCAESFKEFSANNIRDTFRLMLQMAVILTFGGQMPTIKVGRLGGQFAKPRSNPTETRDGVTLPLYTYRGDIINGDTFDEKSRVPDPERLIKAYNQSASTLNLLRGFTHGGFADLQRVTQWNLDFLRHSTQGDRYLELSQRVQDAVGFMAAAGLTTRHPIMTTAELWTSHECLHLQYEQALTREDSISGMYYDCSGHMLWVGERTRQLDGAHVEFLRGISNPLGVKVSDKLDPSELVKLCEILNPHNKPGRLTIITRMGAENMRVKLPHMIGAVRQAGMIVTWVSNPMHGNTIRAPCGLKTRSFDATRAFFDVHEQEGSHPGGVHLEMTGQDVTECIGGSKAVTFDDLGDRYHTHCDPRLNASQSLELAFAIADRLRKKRNMTWNKLMSRAIA

SEQ ID NO: 32 - DNA, 1470 bp, Setaria viridis
  source 1..1470: mol_type = other DNA
atgtattgcatgcgaagcgtttcgcgcgccgcgcccgcctcctttcctctcgcggttcgctccccccgattcgatctcccctccctgcccaccgcgccgccggctccccccagtgccccacggctcctcgtgccgcagacgaggccgcggcggggccccacgatgtaccctgacccggccgcgctggaggccacggagcgcgcgctggagacgttcccgccgctggtgttcgcgggcgaggcgcggaagctggaggagcggctcggggaggcgttcctcctgcagggcggcgactgcgccgagagcttcaaggagttcagcgccaacaacatccgcgacacgttccggctcatgctgcagatggccgtcatcctcaccttcggcggccagatgcccaccatcaaggttggaaggttgggtggtcaatttgctaaaccaagatcgaacccaactgagactagagatggggtaacgcttcccctctatacctatagaggggatatcatcaatggggacacttttgatgagaaatcacgtgtaccagatcctgaaaggctgattaaggcctacaaccagtcagcaagcacactgaatcttctcagaggatttacccatggagggtttgcagatcttcagagagtcacccagtggaatctcgatttcttgaggcacagcacacaaggagacaggtatctggagctttctcagagggttcaggatgccgttgggtttatggctgctgctggtctgactactcgacaccccatcatgaccacagctgagttatggacatcccatgagtgccttcacttacaatatgagcaagcactgactagggaggactccatttctgggatgtactatgactgttctggacacatgctttgggtcggtgagaggactaggcagctggatggtgctcatgttgaattccttcgtggtatttcaaatcctctgggtgtaaaggtaagtgataagcttgatccatcagagctggtgaagttgtgtgagattctgaaccctcacaacaagcccggtagattgaccattatcacaagaatgggggctgagaacatgcgtgtcaagcttccacatatgatcggagcagttcgccaagctgggatgattgttacttgggttagcaatccaatgcacgggaacaccatcagggcaccgtgtgggctgaagacaagatcattcgatgcaaccagggctttctttgatgttcatgagcaagagggaagccaccccggaggtgttcacttggagatgacggggcaggacgttacagaatgtattggtggatccaaggcagtgacatttgatgatctcggtgatcgctaccacacacactgtgaccccaggctgaacgcatcgcagtccctcgagctggcttttgccattgctgacaggctaaggaagaagagaaacatgacatggaacaaattgatgtccagggctattgcttaa

SEQ ID NO: 33 - protein, 387 aa, Brachypodium distachyon
MLSSSTSHLRLHQPSRAARRLPPAPAPATYQFQSPASRRSLPAPAVGARIRPNRGGTIRAIDAAQPFDYESRAAGLLEERQRLKIAIVGFGNFGQFLARTFARQGHTLLAHSRSDHSSLAASLGAAYFQDPHDLCECHPDVVLLATSILSAEAVLRSLPVHRLRRNTLFVDVLSVKEFPKNLLLTTLPEGFDIICTHPMFGPESARDGWDGLPFVFDKVRVGDCPARRARADTFLNIFEREGCRMVEMSCAEHDAHAAETQFLTHTVGRMLATLELQSTPINTKGYETLLRLVDNTCSDSFDLYNGLFMYNKNSTDLLNRLESAMDSVKKRLFDGLHDVLRKQLFEGKASPPATSSNTKSDVHRGQLLLEGKASPAPLNANNTTVRN

SEQ ID NO: 34 - DNA, 1164 bp, Brachypodium distachyon
  source 1..1164: mol_type = other DNA
atgctgtcgtcttccacctcccacctccgcctccaccaaccatccagagcggcgcgccgcctcccgccggctcctgctccggcgacctaccagttccagtcgcccgcatcccgtcgctccctccccgcgccggccgtcggcgcccgcatccgccccaaccgcggcggcaccatccgcgccatcgacgccgcccagccgttcgactacgagtcccgcgcggcggggctgctggaggagcggcagcgcctgaagatcgccatcgtcgggttcggcaacttcgggcagttcctggcccggaccttcgcgcggcagggccacaccctgctcgcccactcccgctccgaccactcctccctcgccgcctccctgggcgccgcctacttccaggacccgcacgacctctgcgagtgccacccggacgtggtcctcctggccacctccatcctctccgccgaggccgtcctccgctcgctccccgtccaccgcctccgccgcaacaccctcttcgtcgacgtcctctccgtcaaggaattccccaagaacctcctcctcaccacgctcccggagggcttcgacatcatctgcacccaccccatgttcggcccggagtcggcccgcgacggctgggacggcctccctttcgtcttcgacaaggtccgcgtcggcgactgccccgcccgccgcgcccgcgccgacaccttcctcaacatcttcgagcgcgagggctgccgcatggtggagatgtcctgcgccgagcacgacgcgcacgccgccgagacgcagttcctcacgcacaccgtcggccgcatgctcgccacgcttgagctccagtccacccccatcaacaccaagggttacgagacgctgctccgcctcgtcgacaacacctgcagcgacagcttcgacctctacaacggactcttcatgtacaacaagaactccaccgacctgctcaaccgcctcgagtccgccatggattccgtcaagaagaggctcttcgacggcctccacgacgtgctccggaagcagctcttcgagggcaaggcgtcgccgcccgccaccagcagcaacaccaaatctgacgtgcaccgggggcagctcctcttggagggcaaggcctcgccggcgccgctcaacgccaacaacaccaccgtccggaattag

SEQ ID NO: 35 - protein, 365 aa, Brachypodium distachyon
MSSSGRFHQPPSCRRPAALAQSLRASTTTVTCRWRQQYHHPLAAAPLRLRAVPVRATDAARQPFDHLSGAVKSEEGTHPRLKIAIVGFGNYGQFLARTMVQQGHTVLAHSRSDHSAAAATIGASFYADAHDLCECQPDVVLLSTSILSAEAVLRSLPVHRFRRSTLFADVLSVKEFPKNLLLAYLPGDFDVICTHPMFGPESARDGWAGLPFVFDEVRVGDGPARRARADAFLDVFAREGCRMVEMSCAEHDAHAAETQFLTHTVGRMLATLDLKSTPINTKGYETLLRLVDNTCSDSFDLYNGLFMYNNNATELLHRLESAMDSVKRRLFDGLHEVLRRQLFEGSPPLNRDSSFPAESSLDDGR

SEQ ID NO: 36 - DNA, 1098 bp, Brachypodium distachyon
  source 1..1098: mol_type = other DNA
atgtcttcctccggtcgcttccaccaacccccttcctgccgacggccggcggctctagcccagtctctacgcgcgtccaccaccacggtgacatgccggtggcggcagcagtaccatcatcccctagctgctgctccgctccgcctccgcgctgtgcctgtgcgtgccacggacgccgcacgccaaccgttcgaccatttgtccggcgccgttaaaagcgaggagggcacgcatccgcgcctcaagatcgccatcgtcgggttcggcaactacggccagtttctggcgcggacgatggtgcagcaggggcacaccgtgctggcccactcccgctccgaccactccgccgccgcggccaccatcggcgcgtccttctacgccgacgcgcacgacctctgcgagtgccagcccgacgtggtcctcctatccacctcgatcctctccgcggaggccgtcctccgctcgctccccgtccaccgcttccgccgcagcaccctcttcgccgacgtcctctccgtcaaggagttccccaagaacctcctcctcgcctacctcccgggggacttcgacgtgatctgcacccaccccatgttcggcccggagtcggcccgcgacggctgggccgggctccccttcgtcttcgacgaggtccgcgtcggcgacggcccggcccgccgcgcccgcgccgacgccttcctcgacgtcttcgcgcgcgagggctgccgcatggtggagatgtcctgcgcggagcacgacgcgcacgccgccgagacgcagttcctgacgcacaccgtcggccggatgctcgccacgctggacctcaagtccacgccgatcaacaccaagggatacgagacgctgctccgcctcgtcgacaacacctgcagcgacagcttcgacctctacaacggcctcttcatgtacaacaacaacgccacggagctgctccaccgcctggaatccgccatggactccgtcaagaggaggctcttcgacggcctccacgaggtgctcaggaggcagctcttcgaaggctcgccgccgttgaacagggattcctctttcccagccgagtcatcgttggatgacggacggtga

SEQ ID NO: 37 - protein, 361 aa, Brachypodium distachyon
MASSLVHLSSPAGGRTAAAPPSLLSRFSPNFCAFATLRPGPTRPTAATPKHARARTCAEQEQKQEVAAPCRDAYEKPAVWNTTAADSAEAAPPLRVGIIGFGNFGQFIARGIQRQGHAVLATSRSDYSAYCSAQGIRYFRSLEALCEEQPNVLLVCSSILSTEAVVRAIPFHKLRSDTIVADVLSVKQFPRNLLLEILPPEFGIVCTHPMFGPESGKHGWSTLPFVYDKVRLADKGDQKANCGQFLSIFEGEGCRMVEMSCAEHDRHAAASQFITHTIGRVLAQLNLKSTPINTKGFEALLKLTENTVSDSFDLYYGLFMYNVNATEQIEKLERAFEKVKQMLYGRLHDILRKQIVERVPI

SEQ ID NO: 38 - DNA, 1086 bp, Brachypodium distachyon
  source 1..1086: mol_type = other DNA
atggcttcctcccttgtccacctaagctcccccgccggtggccgcaccgccgccgcgccgcccagcttgctaagccgcttcagccccaacttctgcgccttcgctacactccgcccaggcccgaccagaccgaccgccgccacccccaagcacgcgagagccaggacctgcgccgagcaggagcaaaagcaggaggtcgctgccccgtgccgtgacgcctacgagaagccggccgtatggaatacgaccgcggcagactccgcggaggcggcgccgccgctacgcgtggggatcatcggcttcggcaacttcgggcagttcatcgccaggggcatccagcgccagggccatgccgtgctggccacttccagatctgactactccgcctactgctccgcccaagggattcgctacttcaggagcttggaggcgctgtgcgaggagcagcccaacgtgctgctggtgtgcagctcaatcctctcgacggaggccgtcgtccgggcaatccccttccacaagctccgctctgacaccatcgtagctgacgtgctctccgtcaagcagtttccccgtaacctcctcctcgagatcctgccgccggagtttgggatcgtctgcacacaccccatgtttgggccggagagtggtaagcatggctggagcacactgcccttcgtctatgacaaggttcgccttgcggacaaaggagatcagaaagccaattgcggccagttcttgagcatctttgagggagagggatgtcggatggtggagatgtcatgcgcagaacatgatcgccacgctgcggcaagtcaattcatcacacacactattgggagggttctggcgcaactaaatctcaagtccacgccaatcaacaccaaaggttttgaggccctcctgaaacttacagaaaacaccgtgagcgatagtttcgatctatactacgggctcttcatgtacaatgtgaatgccacagagcagattgagaaactggaaagggcatttgagaaggtgaagcagatgctgtatggtagactccatgacatactaagaaagcagatcgtagagagggtccccatttaa

SEQ ID NO: 39 - protein, 362 aa, Sorghum bicolor
MLSSSTSTLRLHQPTRPHRHHPPAPAAGGATHLASPRRWRGHAPGASSPPALRARAQRIRALDAAQPFDFESRAAGLLEERQRLKIAIVGFGNFGQFLARTFARQGHTLLAHSRTDHSALASTLGASFFTDPHDLCECHPDVVLLATSILSAEAVLRSLPVHRLRRNTLFVDVLSVKEFPRNLLLSSLPPDFDVICTHPMFGPESARDGWDGLPFVFDKVRVGDCPARRARAEAFLNIFEREGCRMVEMSCAEHDAHAAETQFLTHTVGRMLAMLELRSTPINTKGYETLLRLVDNTCSDSFDLYNGLFMYNKNSTELLNRVEWAMDSVKKKLFDGLHDVLRKQLFEGSPHAPNVSSSNVRK

SEQ ID NO: 40 - DNA, 1089 bp, Sorghum bicolor
  source 1..1089: mol_type = other DNA
atgctgtcgtcttccacctccaccctccgcctccaccaaccaacccgtccccaccgccaccacccgccggccccggccgccgggggcgccacccacctagcctccccgcggcggtggcgcggccacgccccgggggcgtcgtccccgccggcgctccgcgcacgggcccagcgcatccgcgcgctggacgccgcccagccgttcgacttcgagtcccgcgcggcggggctgctggaggagcgtcagcgcctgaagatcgccatcgtcgggttcggcaacttcgggcagttcctggcgcgcaccttcgcgcggcaggggcacacgctgctcgcccactcccgcaccgaccactccgcgctggcgtccacgctgggggcctccttcttcaccgacccgcacgacctgtgcgagtgccacccggacgtggtgctcctcgccacctccatcctctccgcggaggccgtgctccgctcgctgcccgtccaccgtctccgccgcaacacgctcttcgtcgacgtgctctccgtcaaggagttccccaggaacctgctgctcagctcgttaccgcccgacttcgacgtcatctgcacccaccccatgttcgggccggagtcggcgcgcgacggctgggacggccttcccttcgtgttcgacaaggtgcgcgtgggcgactgcccggcccgccgcgcccgcgccgaggcgttcctcaacatcttcgagcgggaagggtgccggatggtggagatgtcgtgcgccgagcacgacgcgcacgccgccgagacccagttcctgacgcacaccgtcgggaggatgctcgccatgctggagctccgctccacgcccatcaacaccaaggggtacgagacgctgctccgcctggtcgacaacacctgcagcgacagcttcgacctctacaacgggctcttcatgtacaacaagaactccaccgagctgctcaaccgcgtcgagtgggccatggactccgtcaagaagaagctcttcgacggcctccacgacgtgctccggaagcagctcttcgaggggtcgccgcacgcccccaatgtctcctcctccaacgtccgcaagtaa

SEQ ID NO: 41 - protein, 389 aa, Sorghum bicolor
MASSSCVRLHHLPSTSRRATAPTNLQFRAASCRWRRLSIPVPGVSPPPLRLRATGASQPLGTDSNEEVEKVEEQPQPPPRLKIAVVGFGTFGQFLARTLVAQGHTVLAHSRSDHSAAAASMGALFFSDPHDLCECHPDVVLLATSILSAESVVRSLPLHRLRRDTLFADVLSVKEFPKRLLLGLLPEEMDILCTHPMFGPESARAGWAGLPFMFDKVRVRDTVPARRARAEAFLDVFAQEGCRMVEMSCAEHDAHAAETQFLTHTVGRMLAALELRATPIDTRGYETLLRLVENTCSDSFDLYNGLFMYNNNSTELLNRLDWAMDAVKRRLFDGLHDVLRRQLFHVGEVEGEAERMEELAVVPGGGPDTDDDGCATATATSIKSGEENN

SEQ ID NO: 42 - DNA, 1170 bp, Sorghum bicolor
  source 1..1170: mol_type = other DNA
atggcgtcatcttcctgcgtccgcctccatcacctgccgtccacctctcgccgggcgaccgctccaaccaaccttcagttccgcgcggcgagctgccggtggcggcggctttccattcccgttcccggcgtgtcccctcctcccctccgcctccgcgccacgggtgcctcgcagccgcttggtactgacagcaacgaggaggtcgagaaggtggaggagcagcctcagcctccgccgcggctcaagatcgccgtggtcgggttcggcaccttcggacagttcctggcgcgcacgctggtggcgcaggggcacacggtgctggcgcactcccggtccgaccactccgccgcggcggcgtccatgggcgcgctcttcttctcggacccgcacgacctgtgcgagtgccacccggacgtggtgctcctggccacctccatcctgtccgcggagtccgtggtccggtcgctcccgctgcaccgcctccgccgcgacaccctcttcgccgacgtgctctccgtgaaggagttccccaagaggctcctcctgggcttgctcccggaggagatggacatcctctgcacgcacccgatgttcgggccggagtcggcgcgcgccggctgggcgggcctccccttcatgttcgacaaggtgcgcgtccgggacaccgtcccggcgcgccgcgcccgcgccgaggcgttcctggacgtgttcgcgcaggaagggtgccggatggtggagatgtcgtgcgccgagcacgacgcgcacgccgccgagacgcagttcctgacgcacaccgtcggtaggatgctggcggcgctggagctccgggcgacgccgatcgacacgagagggtacgagacgctgctccggctggtggagaacacgtgcagcgacagcttcgacctctacaacggcctgttcatgtacaacaacaactccaccgagctgctcaaccgcctcgactgggccatggacgccgtcaagaggaggctcttcgacggcctccacgacgtgctccggaggcagttgttccacgtcggggaggtggagggggaggcggagcgcatggaggagctggcggtggtgccgggtggtggtccggacaccgatgacgacgggtgcgcgacggcgacggcgacgagcatcaagtcgggagaggagaataattga

SEQ ID NO: 43 - protein, 382 aa, Sorghum bicolor
MASSLRHFAGLGCFPAVASTSGSTCCLRRYTPNFCAFVALRPISPPAATATATAKALTSPSPVEQHLQAVVPCHGISDPPAASSAAAVPAAPLRVGIVGFGNFGQFIAGGLQRQGHVVLAASRSDYSVYCASHGIRFFRSVDALCEEQPDVLLICSSILSTEGVVRAIPFRKLRHDTIVADVLSVKEFPRNLLLEVLPPGFGIICTHPMFGPESGKHGWGKLPFVFDKVRVAEDGDQAAKCDQFLSIFEQEGCRMVEMSCAEHDRYAAGSQFITHTIGRVLSQLNLKSTPINTKGYETLLQLTKNTVSDSFDLYYGLFMYNVNATEQLDKLEMAFEKVRQMLSGRLHDFIRKQIVERAAHVPADPSGKLANGLSSSPAARLL

SEQ ID NO: 44 - DNA, 1149 bp, Sorghum bicolor
  source 1..1149: mol_type = other DNA
atggcctcctcgctccgccatttcgccggcctcggctgtttccccgccgtggcctctacctctggctccacctgctgcctgcgccgctacaccccaaacttctgcgccttcgtggcgctccgtccgatcagcccgcccgccgccaccgccacggcgactgccaaggccctcacctctccgtctccggtggagcagcacctacaggccgtcgtcccgtgccacggcatcagcgacccgccggcggcgtcatcggcagcagcggtcccggcggcgccgctgcgggtggggatcgtcgggttcggcaacttcgggcagttcatcgcgggcgggctgcagcggcagggccacgtcgtgctggcggcttccagatccgactactccgtctactgcgccagccatggcattcgcttcttcaggagcgtcgacgcgctgtgcgaggagcagccggacgtgctgctcatctgcagctccatcctgtccacggagggcgtcgtccgagccatccccttccgcaagctccgccacgacaccatcgtcgccgacgtgctctccgtcaaggagttccctcgcaacctcctcctcgaggttctcccaccgggatttgggatcatctgtacgcaccccatgtttgggccggagagtggtaaacacggctggggcaagcttcccttcgtctttgacaaggtccgtgtcgcggaagacggggatcaggcagcaaagtgcgaccagttcttgagcatatttgaacaggagggatgtaggatggtggagatgtcatgcgcggagcatgatcgctacgctgcgggaagtcaattcatcacgcacacaattgggagggttttatcacaactgaacctcaagtcaacgccaatcaacaccaagggttatgaaaccttgctgcaacttaccaagaacaccgtaagcgacagtttcgatctgtactatgggctcttcatgtacaatgtcaatgccacagagcagctcgacaaactggaaatggcatttgagaaggtgagacagatgctgtctggcaggctccacgacttcatacgaaagcagattgtggagagggcagcccatgtgccagcagatccttcaggaaaattggcaaatggtttgtccagttctcctgcagctcgcctcttatag

SEQ ID NO: 45 - DNA, 35 bp, synthetic construct
  source 1..35: mol_type = other DNA
gatctagactcgagggtaccatgctgtcgtcttcc

SEQ ID NO: 46 - DNA, 31 bp, synthetic construct
  source 1..31: mol_type = other DNA
ctagtgcatgcggccgcattccggacggtgg

SEQ ID NO: 47 - DNA, 31 bp, synthetic construct
  source 1..31: mol_type = other DNA
cgcgcggcagccatatgcgcgccatcgacgc

SEQ ID NO: 48 - DNA, 33 bp, synthetic construct
  source 1..33: mol_type = other DNA
gctcgaattcggatccctaattccggacggtgg

SEQ ID NO: 49 - DNA, 36 bp, synthetic construct
  source 1..36: mol_type = other DNA
gatctagactcgagggtaccatgtcttcctccggtc

SEQ ID NO: 50 - DNA, 32 bp, synthetic construct
  source 1..32: mol_type = other DNA
ctagtgcatgcggccgcccgtccgtcatccaa

SEQ ID NO: 51 - DNA, 31 bp, synthetic construct
  source 1..31: mol_type = other DNA
cgcgcggcagccatatgcgtgccacggacgc

SEQ ID NO: 52 - DNA, 32 bp, synthetic construct
  source 1..32: mol_type = other DNA
gctcgaattcggatcctcaccgtccgtcatcc

SEQ ID NO: 53 - DNA, 36 bp, synthetic construct
  source 1..36: mol_type = other DNA
gatctagactcgagggtaccatggcttcctcccttg

SEQ ID NO: 54 - DNA, 33 bp, synthetic construct
  source 1..33: mol_type = other DNA
ctagtgcatgcggccgcaatggggaccctctct

SEQ ID NO: 55 - DNA, 32 bp, synthetic construct
  source 1..32: mol_type = other DNA
cgcgcggcagccatatggccgagcaggagcaa

SEQ ID NO: 56 - DNA, 34 bp, synthetic construct
  source 1..34: mol_type = other DNA
gctcgaattcggatccttaaatggggaccctctc

SEQ ID NO: 57 - DNA, 39 bp, synthetic construct
  source 1..39: mol_type = other DNA
tcactctgtggtctcaaatgcgcgcgctggacgccgccc

SEQ ID NO: 58 - DNA, 41 bp, synthetic construct
  source 1..41: mol_type = other DNA
ccacttcgtggtctcacgaactcttgcggacgttggaggag

SEQ ID NO: 59 - DNA, 39 bp, synthetic construct
  source 1..39: mol_type = other DNA
tcactctgtggtctcaaatgcgcgccacgggtgcctcgc

SEQ ID NO: 60 - DNA, 44 bp, synthetic construct
  source 1..44: mol_type = other DNA
ccacttcgtggtctcacgaactattattctcctctcccgacttg

SEQ ID NO: 61 - DNA, 40 bp, synthetic construct
  source 1..40: mol_type = other DNA
tcactctgtggtctcaaatgagcccgcccgccgccaccgc

SEQ ID NO: 62 - DNA, 42 bp, synthetic construct
  source 1..42: mol_type = other DNA
ccacttcgtggtctcacgaacttaagaggcgagctgcaggag

SEQ ID NO: 63 - DNA, 19 bp, synthetic construct
  source 1..19: mol_type = other DNA
atggcctcctcgctccgcc

SEQ ID NO: 64 - DNA, 20 bp, synthetic construct
  source 1..20: mol_type = other DNA
gtatccggttgaagtgtagg

SEQ ID NO: 65 - DNA, 20 bp, synthetic construct
  source 1..20: mol_type = other DNA
caccaccgtccggaattagc

SEQ ID NO: 66 - DNA, 20 bp, synthetic construct
  source 1..20: mol_type = other DNA
gcaccagtttctccccaaag

SEQ ID NO: 67 - DNA, 20 bp, synthetic construct
  source 1..20: mol_type = other DNA
gatgacggacggtgatctcg

SEQ ID NO: 68 - DNA, 20 bp, synthetic construct
  source 1..20: mol_type = other DNA
ttcgtaccgcttgttggtcg

SEQ ID NO: 69 - DNA, 19 bp, synthetic construct
  source 1..19: mol_type = other DNA
tgctgtgtcccctctcctc

SEQ ID NO: 70 - DNA, 20 bp, synthetic construct
  source 1..20: mol_type = other DNA
agggctgaaagacactgggc

SEQ ID NO: 71 - DNA, 21 bp, synthetic construct
  source 1..21: mol_type = other DNA
cagacaatgcggagatgatcg

SEQ ID NO: 72 - DNA, 22 bp, synthetic construct
  source 1..22: mol_type = other DNA
tttgcttcagaaaccatgtcac

SEQ ID NO: 73 - DNA, 21 bp, synthetic construct
  source 1..21: mol_type = other DNA
ggtagtaattcagtgcgtcgg

SEQ ID NO: 74 - DNA, 21 bp, synthetic construct
  source 1..21: mol_type = other DNA
ggtgttcttcttccagagagg

SEQ ID NO: 75 - DNA, 20 bp, synthetic construct
  source 1..20: mol_type = other DNA
gatcgcttccatcccaaggc

SEQ ID NO: 76 - DNA, 20 bp, synthetic construct
  source 1..20: mol_type = other DNA
caggcggtctgaaaggaagg

SEQ ID NO: 77 - DNA, 20 bp, synthetic construct
  source 1..20: mol_type = other DNA
agttgtcgcgtgtctgagtc

SEQ ID NO: 78 - DNA, 21 bp, synthetic construct
  source 1..21: mol_type = other DNA
acacgggctcacttattcatc

SEQ ID NO: 79 - DNA, 20 bp, synthetic construct
  source 1..20: mol_type = other DNA
gggctcattgtgctgctgtc

SEQ ID NO: 80 - DNA, 20 bp, synthetic construct
  source 1..20: mol_type = other DNA
ccggaggacataggacttgc

SEQ ID NO: 81 - DNA, 42 bp, synthetic construct
  source 1..42: mol_type = other DNA
ggtgccgcgcggcagccatatggccgtccacgccgcggagcc

SEQ ID NO: 82 - DNA, 43 bp, synthetic construct
  source 1..43: mol_type = other DNA
cggagctcgaattcggatcctcagaaaccataggttggcaatg

SEQ ID NO: 83 - DNA, 42 bp, synthetic construct
  source 1..42: mol_type = other DNA
ggtgccgcgcggcagccatatggccgtgcacgccgccgaccc

SEQ ID NO: 84 - DNA, 43 bp, synthetic construct
  source 1..43: mol_type = other DNA
cggagctcgaattcggatccttagaaggccaatggcggcagtg

SEQ ID NO: 85 - DNA, 42 bp, synthetic construct
  source 1..42: mol_type = other DNA
ggtgccgcgcggcagccatatgatccgcgcgcacgcggtgcg

SEQ ID NO: 86 - DNA, 41 bp, synthetic construct
  source 1..41: mol_type = other DNA
cggagctcgaattcggatcctcagagtcccatggatgatgg

SEQ ID NO: 87 - DNA, 42 bp, synthetic construct
  source 1..42: mol_type = other DNA
ggtgccgcgcggcagccatatgcgcgcgacgtcggtcgcggc

SEQ ID NO: 88 - DNA, 46 bp, synthetic construct
  source 1..46: mol_type = other DNA
cggagctcgaattcggatccttaagcttctactctagatatcaagc

SEQ ID NO: 89 - DNA, 42 bp, synthetic construct
  source 1..42: mol_type = other DNA
ggtgccgcgcggcagccatatggccatccacgccgccgaccc

SEQ ID NO: 90 - DNA, 42 bp, synthetic construct
  source 1..42: mol_type = other DNA
cggagctcgaattcggatcctcagaaagccagtggtggcagc

SEQ ID NO: 91 - DNA, 41 bp, synthetic construct
  source 1..41: mol_type = other DNA
ggtgccgcgcggcagccatatgctccgcgcccgcgccgtcc

SEQ ID NO: 92 - DNA, 42 bp, synthetic construct
  source 1..42: mol_type = other DNA
cggagctcgaattcggatcctcacagacgaatggaacccagc

SEQ ID NO: 93 - DNA, 36 bp, synthetic construct
  source 1..36: mol_type = other DNA
tcactctgtggtctcaaatggccgtgcacgccgccg

SEQ ID NO: 94 - DNA, 38 bp, synthetic construct
  source 1..38: mol_type = other DNA
ccacttcgtggtctcacgaactgaaggccaatggcggc

SEQ ID NO: 95 - DNA, 40 bp, synthetic construct
  source 1..40: mol_type = other DNA
tcactctgtggtctcaaatggccgtccacgccgcggagcc

SEQ ID NO: 96 - DNA, 42 bp, synthetic construct
  source 1..42: mol_type = other DNA
ccacttcgtggtctcacgaactgaaaccataggttggcaatg

SEQ ID NO: 97 - DNA, 36 bp, synthetic construct
  source 1..36: mol_type = other DNA
tcactctgtggtctcaaatgatccgcgcgcacgcgg

SEQ ID NO: 98 - DNA, 39 bp, synthetic construct
  source 1..39: mol_type = other DNA
ccacttcgtggtctcacgaactgagtcccatggatgatg